SEQUENCE LISTING (1 sequence)

SEQ ID NO: 1 - protein, 5 aa, Artificial Sequence
  linker
  REPEAT (1)..(5): repeat up to 3 times
GGGGS

(19) United States
(12) Patent Application Publication
(10) Pub. No.: US 2022/0119476 A1
(43) Pub. Date: Apr. 21, 2022

(54) Activation of Antigen Presenting Cells and Methods for Using the Same

(71) Applicant: THE TRUSTEES OF THE UNIVERSITY OF PENNSYLVANIA, Philadelphia, PA (US)

(72) Inventors: Saar Gill, Philadelphia, PA (US); Michael Klichinsky, Philadelphia, PA (US)

(21) Appl. No.: 17/272,000

(22) PCT Filed: Aug. 30, 2019
(86) PCT No.: PCT/US2019/048989

§ 371 Date: Feb. 26, 2021

Related U.S. Application Data
(60) Provisional application No. 62/725,475, filed on Aug. 31, 2018.
(60) Provisional application No. 62/828,843, filed on Apr. 3, 2019.

(51) Int. Cl. C07K 14/725; A61K 35/15; A61P 35/00; C07K 16/28

(52) U.S. Cl. CPC C07K 14/7051; C07K 16/2803; A61P 35/00; A61K 35/15

(57) ABSTRACT

The present invention includes methods and compositions for enhancing antigen presentation in a cell. Antigen presenting cells (APCs) are transformed such that a transformed antigen presenting cell includes at least one exogenous nucleic acid molecule encoding a chimeric antigen receptor (CAR); wherein transforming results in an increase in the antigen presenting ability of the cell as compared to a cell of the same type not having been so transformed. Other aspects of this invention include methods for converting one or more endogenous APCs to a classically activated phenotype and methods of killing tumor cells in a patient, by transforming one or more APCs and administering them to the patient.

### CROSS-REFERENCE TO RELATED APPLICATIONS

The present application claims priority under 35 U.S.C. § 119(e) to U.S. Provisional Patent Application No. 62/725,475, filed Aug. 31, 2018 and U.S. Provisional Patent Application No. 62/828,843, filed Apr. 3, 2019, which are incorporated herein by reference in their entirety.

### BACKGROUND OF THE INVENTION

Macrophages are abundant in the tumor microenvironment (TME) of most cancers where they generally adopt an immunosuppressive (M2) phenotype and exert pro-tumoral functions such as invasion and angiogenesis, priming the pre-metastatic niche, facilitating metastasis and immunosuppression. Macrophages in the TME arise from bone marrow-derived monocytes that are recruited by tumor/stromal cell derived chemokines. The fact that tumor-polarized macrophages support cancer growth is highlighted by observations that tumor progression may be halted by inhibition of macrophage survival, infiltration or pro-tumoral cytokine production. The importance of macrophages in the tumor microenvironment has generated interest in therapeutic approaches to (i) deplete immunosuppressive tumor-associated macrophages, or (ii) enhance anti-tumor macrophages; however these approaches have achieved only limited success. The majority of agents in the first group aim to inhibit the recruitment or survival of TAMs (CSF-1R inhibitors, CSF-1 inhibitors, CCL2 inhibitors, CCR2 inhibitors, CXCR1/2 inhibitors), while others aim to repolarize or inhibit their M2 function (CD40 agonists, trabedectin, CpG analogues, IDO inhibitors, JAK/STAT inhibitors). Agents in the second group include CD47/SIRPa inhibitors that block the phagocytic inhibition imposed on TAMs by CD47 overexpressing tumors. Thus, current macrophage-based immunotherapeutic approaches base their mechanism of action on recruitment of tumor-resident macrophages (TAM).

Outside the environment of established tumors, macrophages are potent effectors of the innate immune system and are capable of at least three distinct anti-tumor functions: phagocytosis, cellular cytotoxicity, and antigen presentation to T cells. Although generally unable to proliferate, macrophages are capable of serial phagocytosis as highlighted by the prodigious ability of the mononuclear phagocytic system to clear approximately 2×10¹¹ erythrocytes per day. Macrophages are critical effectors of targeted antibody-based cancer therapy and have numerous anti-tumor and anti-microbial effector functions. In addition, as professional antigen presenting cells, activated macrophages can present and cross-present antigen to CD4+ and CD8+ T cells, leading to an adaptive anti-tumor immune response.

The broad effector functions of macrophages and their capacity for trafficking into tumors and metastatic lesions spurred previous attempts to adoptively transfer high numbers of autologous macrophages via multiple routes of administration to patients with active malignancy. These clinical trials demonstrated the feasibility and safety of infusing ˜3×10⁹ autologous monocyte-derived macrophages but failed to demonstrate significant anti-tumor efficacy. One possibility for the failure of previous efforts is that macrophages, like other immune cells, require genetic manipulation to redirect them toward a tumor-associated antigen.

Beyond the ability of macrophages to phagocytose cells and debris, they are also professional antigen presenting cells (APCs). To generate a broad immune response against cancer it is likely necessary to stimulate T cells with tumor-derived peptides and to provide adequate co-stimulatory signals. Macrophages can provide all of the above.

A need exists in the art for more effective compositions and methods that treat cancers, in particular those that can enhance antigen presentation. The present invention fulfils this need.

### SUMMARY OF THE INVENTION

The present disclosure encompasses, inter alia, the recognition that certain methods and materials as described herein are able to enhance the ability of antigen presenting cells (e.g., dendritic cells, macrophages, and/or B cells) to present antigens to, for example, T cells (e.g. helper T cells and/or cytotoxic T cells).

In one aspect, the present disclosure provides methods for enhancing antigen presentation in a cell, the method comprising: transforming an antigen presenting cell such that the transformed antigen presenting cell includes at least one exogenous nucleic acid molecule encoding a chimeric antigen receptor (CAR); wherein said transforming results in an increase in the antigen presenting ability of the cell as compared to a cell of the same type not having been so transformed, wherein the enhancement of antigen presenting ability is or comprises one or more of: enhanced CD8+ T cell activation, enhanced CD8+ T cell proliferation, enhanced CD8+ T cell activity, enhanced CD4+ T cell activation, enhanced CD4+ T cell proliferation, enhanced CD4+ T cell activity, enhanced NK cell activation, enhanced NK cell proliferation, and enhanced NK cell activity.

In some embodiments, a transformation comprises transduction with a virus or viral vector comprising at least one exogenous nucleic acid molecule encoding a chimeric antigen receptor (CAR).

In some embodiments, a cell is selected from a primary cell, a macrophage, a dendritic cell, a monocyte or a B cell. In some embodiments, a virus or viral vector is an adenovirus, a lentivirus, an adeno-associated virus, or a foamy virus.

In some embodiments, the at least one exogenous nucleic acid molecule encodes at least one domain of a CAR selected from an antigen binding domain, a transmembrane domain, and an intracellular domain. In some embodiments, the at least one exogenous nucleic acid molecule encodes two or more domains of a CAR selected from an antigen binding domain, a transmembrane domain, and an intracellular domain. In some embodiments, the at least one exogenous nucleic acid molecule encodes each of an antigen binding domain, a transmembrane domain, and an intracellular domain of a CAR.

In some embodiments, an antigen binding domain of a CAR comprises an antibody selected from the group consisting of a monoclonal antibody, a polyclonal antibody, a synthetic antibody, a human antibody, a humanized antibody, a single domain antibody, a single chain variable fragment and an antigen-binding fragment thereof. In some embodiments, an antigen binding domain is selected from the group consisting of an anti-CD19 antibody, an anti-HER2 antibody, an anti-mesothelin antibody or a fragment thereof.

In some embodiments, an intracellular domain is or comprises an intracellular domain of a stimulatory or co-stimulatory molecule. In some embodiments, an intracellular domain of a CAR comprises dual signaling domains.

In some embodiments, a method of the present invention further comprises administering transformed cells to a patient in need thereof. In some embodiments, the patient is suffering from one or more of a cancer, a viral infection, a bacterial infection, a parasitic infection, fibrosis, atherosclerosis, and a neurodegenerative disease.

In some embodiments, a cell is induced into an M1 phenotype prior to the transforming step. In some embodiments, a cell is induced into an M0 phenotype prior to the transforming step. In some embodiments, a cell is exhibits an M1 phenotype prior to the transforming step. In some embodiments, a cell exhibits an M0 phenotype prior to the transforming step.

In another aspect, the present disclosure provides pharmaceutical compositions comprising a cell which has been transformed according to any of the methods disclosed herein, wherein the cell exhibits an increase in the antigen presenting ability of the cell as compared to a cell of the same type not having been so transformed, and wherein the enhancement of antigen presenting ability is or comprises one or more of: enhanced T cell activation, enhanced T cell proliferation, and enhanced T cell activity.

In another aspect, the present disclosure provides a method for converting one or more endogenous antigen presenting cells (APCs) to a classically activated phenotype. The method comprises at least one of exposing the one or more endogenous APCs to one or more exogenous APCs that have been transformed such that the transformed APCs include at least one exogenous nucleic acid molecule encoding a chimeric antigen receptor (CAR).

In some embodiments, transformation comprises transduction with a virus or viral vector comprising at least one exogenous nucleic acid molecule encoding a chimeric antigen receptor (CAR) (transduced APCs). In some embodiments, the one or more endogenous APCs comprise monocytes, macrophages and/or dendritic cells. In some embodiments, the one or more transformed exogenous APCs comprise macrophages. In some embodiments, the classically activated phenotype comprises macrophages exhibiting an M1 phenotype. In some embodiments, at least some of the endogenous macrophages exhibited an M2 phenotype prior to the exposing step.

In some embodiments, the classically activated phenotype comprises increased expression of one or more genes associated with interferon signaling, neuroinflammation signaling, Th1 development, iNOS signaling, death receptor signaling, apoptosis signaling, dendritic cell maturation, inflammasome pathway, activation of IRF by cytosolic pattern recognition receptors, RIG-1-like receptor signaling in antiviral innate immunity, cytotoxic T lymphocyte-mediated apoptosis, JAK1/JAK2/TYK2 interferon signaling, GM-CSF signaling, IL-8 signaling, acute phase response signaling, IL-1 signaling, and/or CD40 signaling.

In some embodiments, the one or more genes involved in interferon signaling are selected from a list comprising, but not limited to BAK1, BAX, BCL2, IFI35, IFI6, IFIT1, IFIT3, IFITM2, IFITM3, IFNAR2, IFNGR2, IRF9, ISG15, OAS1, PTPN2, STAT1, STAT2, and TYK2.

In some embodiments, the one or more genes involved in neuroinflammation signaling are selected from a list comprising, but not limited to ACVR1, APH1A, B2M, BACE2, BCL2, BIRC3, BIRC5, CASP3, CASP8, CCL5, CD80, CFLAR, CREBBP, FAS, FOS, GLS, GLUL, GRIN2D, HLA-A, HLA-DQA1, HLA-E, HLA-F, ICAM1, IFNGR2, IKBKB, IRF7, JAK3, MYD88, NCSTN, NFATC2, PIK3R2, PIK3R5, PLA2G12A, PLA2G4A, PPP3CA, PSEN1, S100B, SLC1A3, STAT1, TBK1, TGFBR1, TRAF3, TYK2, and XIAP.

In some embodiments, the one or more genes involved in Th1 development are selected from a list comprising, but not limited to APH1A, CD274, CD80, HLA-A, HLA-DQA1, ICAM1, IFNGR2, JAK3, MAP2K6, NCSTN, NFATC2, NFIL3, PIK3R2, PIK3R5, PSEN1, RUNX3, SOCS3, STAT1, STAT3, STAT4, and TYK2.

In some embodiments, the one or more genes involved in iNOS signaling are selected from a list comprising, but not limited to CREBBP, FOS, HMGA1, IFNGR2, IKBKB, JAK3, MYD88, STAT1, and TYK2.

In some embodiments, the one or more genes involved in death receptor signaling are selected from a list comprising, but not limited to ACIN1, ACTA2, ACTB, ACTG1, APAF1, ARHGDIB, BCL2, BIRC3, CASP10, CASP2, CASP3, CASP7, CASP8, CFLAR, CYCS, DFFA, FAS, HSPB1, IKBKB, MAP4K4, PARP1, PARP10, PARP12, PARP14, PARP4, PARP6, PARP8, PARP9, SPTAN1, TBK1, TNFRSF21, and XIAP.

In some embodiments, the one or more genes involved in apoptosis signaling are selected from a list comprising, but not limited to ACIN1, APAF1, BAK1, BAX, BCL2, BCL2A1, BCL2L11, BIRC3, CAPNS1, CASP10, CASP2, CASP3, CASP7, CASP8, CDK1, CYCS, DFFA, FAS, IKBKB, MAP4K4, MCL1, MRAS, NRAS, PARP1, PRKCA, RAP1A, RAP2A, SPTAN1, and XIAP.

In some embodiments, the one or more genes involved in dendritic cell maturation are selected from a list comprising, but not limited to B2M, CCR7, CD80, CD83, COL5A3, CREBBP, FCER1G, FCGR1A, FSCN1, HLA-A, HLA-DQA1, HLA-E, HLA-F, ICAM1, IKBKB, IL15, MYD88, PIK3R2, PIK3R5, PLCB3, RELB, STAT1, STAT2, and STAT4.

In some embodiments, the one or more genes involved in the inflammasome pathway are selected from a list comprising, but not limited to AIM2, CASP8, CTSB, MYD88, and NLRP1. In some embodiments, the one or more genes involved in the activation of IRF by cytosolic pattern recognition receptors are selected from a list comprising, but not limited to APAF1, B2M, BCL2, CASP3, CASP7, CASP8, CYCS, DFFA, FAS, FCER1G, HLA-A, HLA-E, and HLA-F.

In some embodiments, the one or more genes involved in the role of RIG-like receptors in antiviral innate immunity are selected from a list comprising, but not limited to CASP10, CASP8, CREBBP, DDX58, DHX58, EP300, IFIH1, IKBKB, IRF7, MAVS, TBK1, and TRAF3.

In some embodiments, the one or more genes involved in cytotoxic T lymphocyte-mediated apoptosis of target cells are selected from a list comprising, but not limited to APAF1, B2M, BCL2, CASP3, CASP7, CASP8, CYCS, DFFA, FAS, FCER1G, HLA-A, HLA-E, and HLA-F.

In some embodiments, the one or more genes involved in the role of JAK1, JAK2, and TYK2 in interferon signaling are selected from a list comprising, but not limited to IFNAR2, IFNGR2, PTPN2, STAT1, STAT2, STAT3, and TYK2.

In some embodiments, the one or more genes involved in GM-CSF signaling are selected from a list comprising, but not limited to BCL2A1, CAMK2B, CCND1, HCK, MRAS, NRAS, PIK3R2, PIK3R5, PIM1, PPP3CA, PRKCB, PTPN11, RAP1A, RAP2A, STAT1, and STAT3.

In some embodiments, the one or more genes involved in IL-8 signaling are selected from a list comprising, but not limited to BAX, BCL2, CCND1, CCND3, CSTB, CXCR1, CXCR2, EIF4EBP1, FOS, GNA12, GNA13, GNB1, GNG12, GNG2, HBEGF, ICAM1, IKBKB, IQGAP1, ITGB5, LASP1, LIMK2, MAP4K4, MRAS, NRAS, PIK3R2, PIK3R5, PLD2, PRKCA, PRKCB, RAC2, RAP1A, RAP2A, RHOA, RHOBTB1, RHOT1, and VEGFA.

In some embodiments, the one or more genes involved in acute phase response signaling are selected from a list comprising, but not limited to CIS, FOS, IKBKB, MAP2K3, MAP2K6, MRAS, MYD88, NRAS, PDPK1, PIK3R2, PTPN11, RAP1A, RAP2A, SERPINE1, SOCS3, and STAT3.

In some embodiments, the one or more genes involved in IL-1 signaling are selected from a list comprising, but not limited to ADCY1, ADCY3, ADCY6, FOS, GNA12, GNA13, GNB1, GNG12, GNG2, IKBKB, MAP2K3, MAP2K6, MRAS, MYD88, PRKAR2A, PRKAR2B, and TOLLIP.

In some embodiments, the one or more genes involved in CD40 signaling are selected from a list comprising, but not limited to FOS, ICAM1, IKBKB, JAK3, MAP2K3, MAP2K6, MAPKAPK2, PIK3R2, PIK3R5, STAT3, TNFAIP3, TRAF1, TRAF3, and TRAF5.

In some embodiments, the increased expression of one or more genes comprises increased expression of one or both of CD80 and CD86.

In some embodiments of the above aspects, the endogenous APCs are or comprise tumor-associated macrophages.

In another aspect, the present disclosure provides a method of killing tumor cells in a patient, the method comprising: transforming one or more antigen presenting cells (APCs), wherein transformed APCs comprise a chimeric antigen rector (CAR), and administering the one or more transformed APCs to a patient; wherein the one or more transformed APCs are able to kill tumor cells in the patient.

In some embodiments, transforming one or more APCs comprises transducing the one or more APCs with a virus or viral vector comprising at least one exogenous nucleic acid molecule encoding a CAR. In some embodiments, the one or more transformed APCs are monocytes, macrophages and/or dendritic cells. In some embodiments, the macrophages exhibit an M1 phenotype after the transformation step. In some embodiments, killing tumor cells in a patient comprises reducing tumor size in the patient. In some embodiments, a tumor microenvironment (TME) in the patient is altered after administration of the one or more transduced APCs to the patient.

In some embodiments, the altered TME comprises one or more of: recruitment of activated myeloid cells, conversion of suppressive macrophages toward classically activated macrophages, recruitment of natural killer (NK) cells, activation of NK cells, recruitment of T cells, activation of T cells, depletion of tumor-associated macrophages, conversion of myeloid-derived suppressor cells (MDSCs), depletion of MDSCs, increased expression of pro-inflammatory cytokines, a decrease in anti-inflammatory cytokines, an increase in pro-inflammatory cells, a decrease in anti-inflammatory cells, and an increased amount of activated dendritic cells, relative to a TME prior to administration of the one or more transduced APCs to the patient.

In some embodiments, the TME is sampled via a process comprising biopsy of a tumor. In some embodiments, the one or more modified APCs are able to kill the tumor cells in the presence of macrophages exhibiting an M2 phenotype. In some embodiments, the one or more modified APCs maintain the ability to kill the tumor cells while in the presence of an inhibitory TME for a period of time. In some embodiments, an inhibitory TME comprises the presence of one or more immunosuppressive cells selected from: tumor-associated macrophages, T_{reg} cells, B_{reg} cells, MDSCs, and cancer-associated fibroblasts.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1I illustrate the finding that CD3ζ-based chimeric antigen receptors direct macrophage phagocytosis. FIG. 1A shows constructs utilized in lentiviral vectors to express CAR-19 variants in THP-1 cells (left). Representative flow cytometry (FACS) plot of CAR-19 expression (post-sort) in genetically labeled red-fluorescent mRFP+ THP-1 macrophages (right). FIGS. 1B-1C show results from in vitro microscopy based phagocytosis assays by indicated THP-1 macrophages against CD19+ K562 target cells (FIG. 1B). Phagocytosis of CD19+ or control CD19-K562 target cells by CAR-19ζ+ THP-1 macrophages (FIG. 1C). Data represent the mean+/−standard error (SEM) of triplicate wells. Statistical significance was calculated via one-way ANOVA with multiple comparisons (FIG. 1B) or two-sided t-test (FIG. 1C), **p<0.01. FIG. 1D shows CAR-19ζ+ THP-1 macrophages were pre-treated with media, cytochalasin-D, blebbistatin, or R406 prior to phagocytosis assay. Data represent the mean+/−SEM of triplicate wells. Statistical significance was calculated via ANOVA with multiple comparisons, ****p<0.0001. FIG. 1E shows results from Luciferase-based killing assay of CD19+K562 cells by untransduced (UTD), CAR-19γ, or CAR-19ζ THP-1 macrophages (E:T=10:1; 48 hrs). Data represent the mean+/−SEM of triplicate wells. Statistical significance was calculated via ANOVA with multiple comparisons, ***p<0.001; ns=non-significant. FIG. 1F shows imaging cytometry of UTD or CAR-19ζ mRFP+ THP-1 macrophages after co-culture with GFP+ CD19+ K562 target cells. FIG. 1G shows key steps of the CAR-19ζ THP-1 macrophage phagocytosis during a 24-hour live cell fluorescent microscopy analysis. FIG. 1H shows a representative image of poly-phagocytic CAR-19ζ THP-1 macrophages from 4-hour co-culture at a 1:1 effector to target ratio. FIG. 1I shows construct diagrams of anti-HER2 and anti-mesothelin CARs (left). In vitro phagocytosis of UTD or CAR-meso-ζ THP-1s against mesothelin+K562 cells (middle), and in vitro phagocytosis of UTD or CAR-HER2-ζ THP-1s against HER2+ K562 cells (right). Data is represented as mean+/−SEM. Statistical significance was calculated via t-test. ****p<0.0001; **p<0.01.

FIGS. 2A-2M illustrate efficient generation of primary human CAR macrophages with Ad5f35 leads to targeted in vitro and in vivo anti-tumor function. FIG. 2A depicts an anti-HER2 CAR construct cloned into pAd5f35 (top). CAR expression in 10 human donors at an MOI of 1×10³ PFU, 48-hours post-transduction (bottom). FIG. 2B shows FACS-based phagocytosis with primary human control (UTD) or anti-HER2 CAR-macrophages against MDA-468 (HER2−) or SKOV3 (HER2+). The percent of GFP+ events within the CD11b+ population was plotted as percentage phagocytosis. Data is represented as mean+/−standard error. Statistical significant between CAR-HER2-zeta and UTD was calculated using ANOVA with multiple comparisons; ****p<0.0001, ns=non-significant. FIG. 2C illustrates results from human macrophages transduced with CAR-HER2-zeta Ad5f35 at MOIs of 0, 100, 500, or 1000 PFU. CAR expression correlated with MOI (left), in vitro phagocytosis against SKOV3 (middle), and in vitro cytotoxicity against SKOV3 at 48 hours (right). Data are represented as mean+/−SEM. Correlation was determined via linear regression and Pearson correlation. FIG. 2D depicts a panel of 10 human cancer cell lines tested for surface HER2 expression (isotype and MDA-468 are negative controls). These cell lines were exposed to CAR-HER2 macrophages. Percent phagocytosis is shown as a heat map, with each column representing a different donor, and cell lines are ordered by HER2-MFI from low-to-high (top to bottom). FIG. 2E shows luciferase+ SKOV3, HTB-20, or CRL-2351 were used as targets in in vitro cytotoxicity assays with control (UTD) or CAR-HER2-zeta (CAR) macrophages at different E:T ratios. Data is shown as mean+/−SEM for triplicate wells. Statistical significance was calculated using ANOVA with multiple comparisons; ****p<0.0001; ***p<0.001; **p<0.01; *p<0.05; ns=non-significant. FIG. 2F: NSGS mice were injected with SKOV3 IP 2-4 hours prior to receiving injections of either PBS, control (UTD) or CAR-HER2 human macrophages IP as shown. FIGS. 2G-2H show tumor burden (FIG. 2G), measured by bioluminescence (total flux), and body weight (FIG. 2H) over 100 days. FIG. 2I shows a Kaplan-Meier survival curve over 100 days. Statistical significance was calculated using Log-Rank Mantel Cox test; ****p<0.0001. FIG. 2J: Female NSGS mice were intravenously injected with SKOV3 and treated with IV macrophages 7 days later as shown. FIGS. 2K-2L show representative images of tumor burden 31-days post treatment (FIG. 2K) and tumor burden (total flux) over time (FIG. 2L). FIG. 2M shows a Kaplan-Meier survival curve. Statistical significance was calculated using Log-Rank Mantel Cox text; **p<0.01.

FIGS. 3A-3J illustrate the finding that adenovirally transduced human macrophages adopt a unique pro-inflammatory M1 phenotype and demonstrate resistance to immunosuppressive cytokines. FIG. 3A depicts hierarchical clustering of differentially expressed genes (DEGs) from RNA extracted from UTD or Ad5f35-CAR-HER2 transduced human macrophages from 4 matched donors, 48 hours post transduction. The heat map shows log₂ fold-change in gene expression relative to UTD. FIG. 3B shows transcriptome-derived principal component analysis clustering from UTD, Ad5f35-empty transduced, Ad5f35-CAR-HER2 transduced, classically-activated M1 or alternatively-activated M2 human macrophages from 5 donors. FIG. 3C is a differential gene expression volcano-plot between UTD and transduced CAR macrophages. Red indicates strongly upregulated interferon-associated genes. FIG. 3D is a table of Ad5f35 induced canonical pathways in human macrophages. FIG. 3E shows results from CFSE labeled T cells cultured alone or at a 1:1 E:T ratio for 5 days with UTD or autologous CAR macrophages in the presence or absence of PHA. Proliferation of CD8 T cells is shown as percent of CFSE(−)CD8(+) T cells, mean+/−SEM. (***p<0.001; ns=non-significant.) FIG. 3F shows results from control or NY-ESO-1 expressing macrophages (No Ag and Ag, respectively), with or without Ad5f35-CAR co-cultured with CTV-labeled anti-NY-ESO-1 T cells. Proliferation of anti-NY-ESO-1 TCR+ CD8+ T cells is shown as mean+/−SEM. Statistical significance was determined using ANOVA with multiple comparisons. ****p<0.0001. FIG. 3G: NSGS mice were IV injected with SKOV3 as shown in FIG. 2M. Seven days later mice were treated with either IV PBS, CAR macrophages (8×10⁶)+/−autologous T cells (3×10⁶), or T cells alone. Tumor burden over time is shown for each mouse. FIG. 3H shows upregulation of CD206 in response to M2-challenge in UTD or CAR macrophages (representative histograms; top panel, % CD206(+) in response to IL-4; bottom panel). Data is shown as mean+/−SEM from triplicate conditions. Statistical significance was calculated with t-test (****p<0.0001; CAR vs. UTD). FIG. 3I illustrates the change in oxygen consumption rate (OCR) upon treatment with IL-4 in UTD or CAR macrophages (representative OCR diagrams, top panel; mean basal OCR; bottom panel). Data is shown as mean+/−SEM from triplicate conditions. Statistical significance was calculated with t-test (***p<0.001; CAR vs. UTD). FIG. 3J shows upregulated genes from UTD or CAR macrophages challenged with M2-cytokines (or control). Venn diagrams show the number of M2-cytokine induced genes in UTD, CAR, or both macrophage types.

FIGS. 4A-4C illustrate human monocyte derived CAR macrophage manufacturing process and purity. FIG. 4A shows an overview of the CAR macrophage 7-day manufacturing process and timeline. FIG. 4B shows relative abundances of granulocytes, monocytes, T cells, NK cells, and B cells in the pre-selection or post-selection positive/negative fractions, as determined by FACS analysis. The post-selection positive fraction was used for macrophage differentiation. FIG. 4C shows the inter-donor variability in viability and leukocyte purity (macrophages, T cells, B cells, neutrophils, and NK cells) at the time of harvest from 6 normal donors for both control (untransduced, or UTD) and CAR macrophages.

FIGS. 5A-5H illustrate expression of adenoviral docking proteins, comparison of Ad5f35 to lentiviral vectors, and HER2 titration. FIGS. 5A-5D show expression of Ad5-docking protein Coxackie-adenovirus receptor (CXADR) and Ad5f35-docking protein CD46 relative to isotype control (unfilled histogram; FIG. 5A and FIG. 5B, respectively). MFI and percent positivity for CXADR (FIG. 5C) and CD46 (FIG. 5D) from 10 donors. Data represents mean+/−SEM. Statistical significance was determined using t-test; ****p<0.0001. FIGS. 5E-5F show results from primary human macrophages transduced with GFP encoding viruses at decreasing dilution factors. Ad5f35, standard 3^{rd} generation VSV-G pseudotyped lentivirus (Wt LV), or Vpx-packaged lentivirus were compared for transduction efficiency (FIG. 5E) and expression intensity (FIG. 5F). FIGS. 5G-5H show increasing amounts of in vitro transcribed HER-2 mRNA were electroporated into GFP+ MDA-468 (HER2−) target cells to generate titrated antigen expression, which was validated by surface anti-HER2 FACS staining (left; bottom histogram shows control cells). These cells were used as phagocytic targets for CAR-HER2 macrophages (right). Data are shown as mean+/−standard error.

FIGS. 6A-6E illustrate the pro-inflammatory phenotype of primary human macrophages after Ad5f35 transduction. FIG. 6A shows gene expression heatmaps of represented co-stimulatory ligands, antigen processing/presentation, and MHC class I/II genes from 3 normal donors as determined by RNA sequencing of control UTD or Ad5f35 transduced CAR macrophages. Expression is normalized to UTD for each gene. FIG. 6B shows confirmation of select M1 genes by RTqPCR from human macrophages transduced with increasing MOIs of Ad5f35-CAR. GAPDH was used as a housekeeping control gene. Data is represented as mean+/−SEM. Statistical significance was calculated using ANOVA with multiple comparisons; ****p<0.0001; ***p<0.001; **p<0.01; *p<0.05; ns=non-significant. FIG. 6C shows surface expression of select human M1 markers (CD80 and CD86) and M2 marker CD163 in response to transduction with increasing MOIs of Ad5f35-CAR by FACS. Data is represented as mean+/−SEM of the mean fluorescent intensity (MFI) of each marker for duplicate wells. FIG. 6D shows surface expression of human M1 markers (CD80 and CD86) and M2 marker CD163 after transduction with equivalent MOIs of control empty-vector Ad5f35 or Ad5f35-CAR. Data is represented as mean+/−SEM of the MFI of each marker for duplicate wells. FIG. 6E shows surface expression of M1 marker CD86 on control UTD or Ad5f35-CAR transduced macrophages from 10 human matched-donors.

FIGS. 7A-7D illustrate that CAR macrophage (CAR-M) cells push M2 macrophages toward M1 polarization. M2 macrophages were challenged with conditioned media generated from control untransduced (UTD) or CAR macrophages (CAR-M). After exposure to control or CAR-M conditioned media, M2 macrophage RNA was collected and subjected to RNA sequencing and bio-informatics analysis. Left-hand graphs of FIGS. 7A-7D show principle component analysis. Right-hand parts of FIGS. 7A-7D show unbiased hierarchical clustering.

FIG. 8 illustrates the expression of many genes that were upregulated and downregulated in M2 macrophages upon treatment with factors secreted from CAR-treated macrophages (*log FC>1, adj. p-val<0.05). The differentially expressed genes (DEG) were analyzed by the Ingenuity Pathway Analysis algorithm.

FIG. 9 is a series of graphs illustrating the induction of human M1 markers (CD80, CD86, HLA Class II) and downregulation of M2 marker TGF-β1 in M2 macrophages exposed to CAR-M. Cells were stained for the indicated surface marker or permeabilized and stained for the indicated cytokine marker followed by flow cytometry analysis. Data are represented as mean+/−SEM.

FIG. 10 illustrates evaluation of an exemplary gene expression profile of CAR-M cells using RT-qPCR. Data are represented as mean+/−SEM. Statistical significance was calculated via t-test. ****p<0.0001; **p<0.01; *p<0.05 for the indicated comparisons of CAR-M vs. UTD samples.

FIG. 11 illustrates the ability of CAR-M to kill SKOV3 tumor cells in the presence of M2 macrophages. SKOV3-GFP cells were seeded in 96-well plate wells with or without untransduced (UTD) and CAR macrophages (30,000 cells) in TexMACS media. Cytotoxicity was monitored on an IncuCyte S3 for subsequent 3 days. Data are represented as mean+/−SEM between sample replicates at the indicated time point.

FIGS. 12A-12B illustrate that CAR-M maintain the ability to kill tumor cells in the presence of a human tumor microenvironment. Cytotoxic ability was assessed in the presence of a single cell suspension of human lung tumor cells. SKOV3-GFP cells were seeded with digested single cell suspensions derived from human lung tumors. Suspensions derived from normal lung tissue and PBMCs were used as controls. UTD or CAR-M cells were then seeded into the mixtures and the cytotoxicity was assessed after 48 hours by the disappearance of GFP fluorescence intensity (FIG. 12A). FIG. 12B is a graph depicting quantification of the data. Data are represented as mean+/−SEM between indicated sample replicates.

FIG. 13 is an illustration depicting an experiment demonstrating that CAR-M cells maintain an M1 phenotype in model tumor microenvironment (TME). NOD scid gamma (NSG) immunodeficient mice were humanized with CD34+ human female hematopoietic stem cells. After engraftment was confirmed, ovarian cancer cells were engrafted subcutaneously in the flank of the mice. After tumor engraftment and growth was visualized, human male control untransduced (UTD) or CAR-macrophages were injected intratumorally. Tumors were harvested and subject to single cell RNA sequencing (scRNA seq) using the 10× genomics pipeline.

FIGS. 14A-14B show a single cell RNA sequencing analysis overlay of control UTD or CAR macrophages after extraction from a tumor xenograft from a humanized mouse. The phenotypes of the control (UTD) and CAR macrophages were directly compared (FIG. 14A). CAR macrophages expressed the CAR (positive control gene, 4D5 scFv). All macrophages expressed CD68, a pan-macrophage marker. Only UTD macrophages expressed the M2 marker MRC1. Only CAR macrophages expressed the M1 markers IFIT1, ISG15, and IFITM1 (FIG. 14B).

FIGS. 15A-15B illustrate results from single cell RNA sequencing of monocytes isolated from humanized mouse model xenografts treated with UTD or CAR-M cells.

FIG. 16 is a series of graphs illustrating the effect of UTD or CAR MAC on the phenotypes of immature and mature dendritic cells. Freshly isolated monocytes were stimulated with GM-CSF and IL-4 for 9 days, followed by maturation with GM-CSF, IL-4, and TNFα for an additional 48 hours. Conditioned media from UTD or CAR macrophages was then added to the cells for 48 hours prior to staining and analysis by flow cytometry. Data are reported as the mean fluorescence intensity (MFI) for the staining of each indicated marker. Error bars are +/−SEM between indicated sample replicates.

### DETAILED DESCRIPTION

### Definitions

Unless defined otherwise, all technical and scientific terms used herein have the same meaning as commonly understood by one of ordinary skill in the art to which the invention pertains. Anyone of skill in the art will understand that methods and materials similar or equivalent to those described herein can be used in accordance with various embodiments. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a monocyte/macrophage/dendritic cell that has been sufficiently stimulated to induce detectable cellular proliferation or has been stimulated to exert its effector function. Activation can also be associated with induced cytokine production, phagocytosis, cell signaling, target cell killing, or antigen processing and presentation.

The term “activated monocytes/macrophages/dendritic cells” refers to, among other things, monocyte/macrophage/dendritic cell that are undergoing cell division or exerting effector function. The term “activated monocytes/macrophages/dendritic cells” refers to, among others thing, cells that are performing an effector function or exerting any activity not seen in the resting state, including phagocytosis, cytokine secretion, proliferation, gene expression changes, metabolic changes, and other functions.

The term “agent,” or “biological agent” or “therapeutic agent” as used herein, refers to a molecule that may be expressed, released, secreted or delivered to a target by the modified cell described herein. The agent includes, but is not limited to, a nucleic acid, an antibiotic, an anti-inflammatory agent, an antibody, an antibody agent or fragments thereof, a growth factor, a cytokine, an enzyme, a protein, a peptide, a fusion protein, a synthetic molecule, an organic molecule (e.g., a small molecule), a carbohydrate or the like, a lipid, a hormone, a microsome, a derivative or a variation thereof, and any combinations thereof. The agent may bind any cell moiety, such as a receptor, an antigenic determinant, or other binding site present on a target or target cell. The agent may diffuse or be transported into the cell, where it may act intracellularly.

The term “antibody,” as used herein, refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention (e.g., as a component of a CAR) include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].

The term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, in some embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody agent may lack a covalent modification (e.g., attachment of a glycan) that it would have if produced naturally. In some embodiments, an antibody agent may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent is not and/or does not comprise a polypeptide whose amino acid sequence includes structural elements recognized by those skilled in the art as an immunoglobulin variable domain. In some embodiments, an antibody agent may be or comprise a molecule or composition which does not include immunoglobulin structural elements (e.g., a receptor or other naturally occurring molecule which includes at least one antigen binding domain).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments and human and humanized versions thereof.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. α and β light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that is capable of provoking an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

As used herein, the term “autologous” is meant to refer to any material derived from the same individual to which it is later to be re-introduced into the individual.

“Allogeneic” refers to a graft (e.g., a population of cells) derived from a different animal of the same species.

“Xenogeneic” refers to a graft (e.g., a population of cells) derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

The term “chimeric antigen receptor” or “CAR,” as used herein, refers to an artificial cell surface receptor that is engineered to be expressed on an immune effector cell and specifically targets a cell and/or binds an antigen. CARs may be used, for example, as a therapy with adoptive cell transfer. Monocytes macrophages and/or dendritic cells are removed from a patient (blood, tumor or ascites fluid) and modified so that they express the receptors specific to a particular form of antigen. In some embodiments, the CARs have been expressed with specificity to a tumor associated antigen, for example. CARs may also comprise an intracellular activation domain, a transmembrane domain and an extracellular domain comprising a tumor associated antigen binding region. In some aspects, CARs comprise fusions of single-chain variable fragments (scFv) derived monoclonal antibodies. CD3-zeta transmembrane domains and intracellular domains. The specificity of CAR designs may be derived from ligands of receptors (e.g., peptides). In some embodiments, a CAR can target cancers by redirecting a monocyte/macrophage expressing the CAR specific for tumor associated antigens.

The term “chimeric intracellular signaling molecule” refers to recombinant receptors comprising one or more intracellular domains of one or more stimulatory and/or co-stimulatory molecules. The chimeric intracellular signaling molecule substantially lacks an extracellular domain. In some embodiments, the chimeric intracellular signaling molecule comprises additional domains, such as a transmembrane domain, a detectable tag, and a spacer domain.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a monocyte/macrophage/dendritic cell, thereby providing a signal which mediates a monocyte/macrophage/dendritic cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a monocyte/macrophage/dendritic cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” or “co-stimulatory domain” refers to a molecule on an innate immune cell that is used to heighten or dampen the initial stimulus. For example, pathogen-associated pattern recognition receptors, such as TLR (heighten) or the CD47/SIRPα axis (dampen), are molecules on innate immune cells. Co-stimulatory molecules include, but are not limited to TCR, CD3 zeta, CD3 gamma, CD3 delta, CD3 epsilon, CD86, common FcR gamma, FcR beta (Fc Epsilon R1b), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combinations thereof.

A “co-stimulatory signal”, as used herein, refers to a signal, which in combination with a primary signal, such as activation of the CAR on a macrophage, leads to activation of the macrophage.

The term “cytotoxic” or “cytotoxicity” refers to killing or damaging cells. In one embodiment, cytotoxicity of the metabolically enhanced cells is improved, e.g. increased cytolytic activity of macrophages.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of monocytes/macrophages. In one embodiment, the monocytes, macrophages, or dendritic cells that are expanded ex vivo increase in number relative to the number originally present in the culture. In another embodiment, the monocytes, macrophages, or dendritic cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses (e.g., Ad5f35)) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. As applied to the nucleic acid or protein, “homologous” as used herein refers to a sequence that has about 50% sequence identity. More preferably, the homologous sequence has about 75% sequence identity, even more preferably, has at least about 80%, 85%, 90%, 91%, 92%, 93%, 94%, 95%, 96%, 97%, 98%, 99% sequence identity.

“Humanized” forms of non-human (e.g., murine) antibodies are chimeric immunoglobulins, immunoglobulin chains or fragments thereof (such as Fv, scFv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), intratumoral (i.t.) or intra-peritoneal (i.p.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combinations thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “resistance to immunosuppression” refers to lack of suppression or reduced suppression of an immune system activity or activation.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

By the term “specifically binds,” as used herein with respect to an antigen binding domain, such as an antibody agent, is meant an antigen binding domain or antibody agent which recognizes a specific antigen, but does not substantially recognize or bind other molecules in a sample. For example, an antigen binding domain or antibody agent that specifically binds to an antigen from one species may also bind to that antigen from one or more species. But, such cross-species reactivity does not itself alter the classification of an antigen binding domain or antibody agent as specific. In another example, an antigen binding domain or antibody agent that specifically binds to an antigen may also bind to different allelic forms of the antigen. However, such cross reactivity does not itself alter the classification of an antigen binding domain or antibody agent as specific. In some instances, the terms “specific binding” or “specifically binding,” can be used in reference to the interaction of an antigen binding domain or antibody agent, a protein, or a peptide with a second chemical species, to mean that the interaction is dependent upon the presence of a particular structure (e.g., an antigenic determinant or epitope) on the chemical species; for example, an antigen binding domain or antibody agent recognizes and binds to a specific protein structure rather than to proteins generally. If an antigen binding domain or antibody agent is specific for epitope “A”, the presence of a molecule containing epitope A (or free, unlabeled A), in a reaction containing labeled “A” and the antigen binding domain or antibody agent, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the Fc receptor machinery or via the synthetic CAR. Stimulation can mediate altered expression of certain molecules, such as downregulation of TGF-beta, and/or reorganization of cytoskeletal structures, and the like.

A “stimulatory molecule,” as the term is used herein, means a molecule of a monocyte, macrophage, dendritic cell that specifically binds with a cognate stimulatory ligand present on an antigen presenting cell.

A “stimulatory ligand,” as used herein, means a ligand that when present on an antigen presenting cell (e.g., an aAPC, a macrophage, a dendritic cell, a B-cell, and the like) or tumor cell can specifically bind with a cognate binding partner (referred to herein as a “stimulatory molecule”) on a monocyte, macrophage, or dendritic cell thereby mediating a response by the immune cell, including, but not limited to, activation, initiation of an immune response, proliferation, and the like. Stimulatory ligands are well-known in the art and encompass, inter alia, Toll-like receptor (TLR) ligand, an anti-toll-like receptor antibody, an agonist, and an antibody for a monocyte/macrophage receptor. In addition, cytokines, such as interferon-gamma, are potent stimulants of macrophages.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

By “target” is meant a cell, tissue, organ, or site within the body that is in need of treatment.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The term “tumor” as used herein, refers to an abnormal growth of tissue that may be benign, pre-cancerous, malignant, or metastatic.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based, in part, on the surprising finding that transformation (e.g., transduction) of antigen presenting cells, for example, with a modified virus (e.g., a virus engineered to express a chimeric antigen receptor) can cause those cells to exhibit enhanced antigen presenting ability. In some embodiments, such enhanced antigen presenting capability is as compared to an antigen presenting cell of the same type not having been so transduced. In some embodiments, enhanced antigen presenting ability is or comprises one or more of: enhanced CD8+ T cell activation, enhanced CD8+ T cell proliferation, enhanced CD8+ T cell activity, enhanced CD4+ T cell activation, enhanced CD4+ T cell proliferation, enhanced CD4+ T cell activity, enhanced NK cell activation, enhanced NK cell proliferation, and enhanced NK cell activity.

In some embodiments, transfection may also provide enhanced antigen presenting capability. In some embodiments, transfection may be or comprise transfection with DNA (e.g. ssDNA, dsDNA), RNA (ssRNA, dsRNA, siRNA, miRNA), artificial nucleic acids (e.g., one or more PNAs) and any combination thereof.

Chimeric antigen receptor (CAR) T cells have generated deep robust responses in patients with hematologic malignancies, but meaningful responses in solid tumors are more elusive. Certain antigen presenting cells, including dendritic cells and macrophages are actively recruited to solid tumors and infiltrate the microenvironment where they can become immunosuppressive and support tumor growth. Measures to recruit macrophage phagocytosis are being actively studied, leading to recent efforts to deplete, repolarize, or disinhibit tumor associated macrophages (TAMs). Given the potential effector functions of macrophages and their capacity for trafficking into tumors, human macrophages were engineered with CARs to genetically direct their anti-tumor function. The use of a chimeric adenoviral vector overcomes the resistance of human macrophages to genetic manipulation and imparts a global pro-inflammatory (M1) phenotype. Collectively, the CAR macrophage platform described herein achieves antigen specificity, anti-tumor activity, and the potential for orchestrating an immune response to metastatic solid tumors. It is specifically contemplated that any type of antigen presenting cell, including dendritic cells, may be enhanced through the methods and compositions described herein.

### Methods

In one aspect, the invention includes methods for enhancing antigen presentation in a cell. In some embodiments, provided methods comprise transforming (e.g., transducing) an antigen presenting cell, for example, with a virus or viral vector comprising at least one exogenous nucleic acid molecule encoding a chimeric antigen receptor (CAR), wherein transforming results in an increase in the antigen presenting ability of the cell as compared to a cell of the same type not having been so transformed, and wherein the enhancement of antigen presenting ability is or comprises one or more of: enhanced T cell activation, enhanced T cell proliferation, and enhanced T cell activity.

In some embodiments, the invention also provides methods for enhancing antigen presentation in a cell, the methods including the step of introducing into an antigen presenting cell, at least one exogenous nucleic acid encoding a chimeric antigen receptor (CAR), wherein said introducing results in an increase in the antigen presenting ability of the cell as compared to a cell of the same type not having been so transduced, wherein the enhancement of antigen presenting ability is or comprises one or more of: enhanced T cell activation, enhanced T cell proliferation, and enhanced T cell activity.

In some embodiments, enhanced T cell activation may be or comprise enhanced activation of one or more of CD8+ T cells, CD4+ T cells, and natural killer (NK) cells. In some embodiments, enhanced T cell proliferation may be or comprise enhanced proliferation of one or more of CD8+ T cells, CD4+ T cells, and natural killer (NK) cells. In some embodiments, enhanced T cell activity may be or comprise enhanced activity of one or more of CD8+ T cells, CD4+ T cells, and natural killer (NK) cells.

In certain embodiments, a cell is selected from a primary cell, a macrophage, a dendritic cell, a monocyte, a B cell, or a stem cell capable of producing one or more of these cell types (e.g., a hematopoietic stem cell, an iPSC). In certain embodiments, a virus or viral vector may be or comprise an adenovirus, a lentivirus, an adeno-associated virus, or a foamy virus.

In certain embodiments, an exogenous nucleic acid molecule encodes at least one domain of a CAR selected from an antigen binding domain, a transmembrane domain, and an intracellular domain. In certain embodiments, an exogenous nucleic acid molecule encodes two or more domains of a CAR selected from an antigen binding domain, a transmembrane domain, and an intracellular domain. In certain embodiments, an exogenous nucleic acid molecule encodes each of an antigen binding domain, a transmembrane domain, and an intracellular domain of a CAR.

In certain embodiments, an antigen binding domain of the CAR is or comprises an antibody selected from the group consisting of a monoclonal antibody, a polyclonal antibody, a synthetic antibody, a human antibody, a humanized antibody, a single domain antibody, a single chain variable fragment and an antigen-binding fragment thereof. In certain embodiments, the antigen binding domain is selected from the group consisting of an anti-CD19 antibody, an anti-HER2 antibody, an anti-mesothelin antibody or a fragment thereof.

In certain embodiments, an intracellular domain is or comprises the intracellular domain of a stimulatory or co-stimulatory molecule. In certain embodiments, the intracellular domain of the CAR comprises dual signaling domains.

In certain embodiments, provided methods further comprise administering the transduced and/or transfected cells to a patient in need thereof. In certain embodiments, a patient is suffering from one or more of a cancer, a viral infection, a bacterial infection, a parasitic infection, fibrosis, atherosclerosis, and neurodegenerative disease.

In certain embodiments, the method further comprises wherein the cell is induced into an M1 phenotype prior to being transformed (e.g., transduced). In certain embodiments, the method further comprises wherein the cell is exhibits an M1 phenotype prior to the transforming step. In certain embodiments, the method further comprises wherein the cell is induced into an M0 phenotype prior to the transforming step. In certain embodiments, the method further comprises wherein the cell exhibits an M0 phenotype prior to the transforming step.

### Antigen Presenting Cells

The present disclosure encompasses methods of transforming (e.g., transducing) one or more antigen presenting cells (e.g., macrophages, dendritic cells, B cells, etc) with at least one exogenous nucleic acid molecule encoding a chimeric antigen receptor (CAR). As used herein, the term “antigen presenting cell” refers to any cell that displays one or more antigens on its surface, for example, in combination with one or more major histocompatibility complex (MHC) proteins. In some embodiments, an antigen presenting cell may be or comprise a macrophage, a dendritic cell, a monocyte, a B cell, or a stem cell.

### Macrophages

Macrophages are immune cells that are specialized for detection, phagocytosis, and destruction of target cells including pathogens and tumor cells. As such, macrophages are potent effectors of the innate immune system and are capable of at least three distinct anti-tumor functions: phagocytosis of dead and dying cells, cytotoxicity against tumor cells themselves, and presentation of tumor antigens to orchestrate an adaptive anti-tumor immune responses. In adult humans, unpolarized, uncommitted, or resting macrophages (M0) differentiate from bone marrow-derived monocyte precursors and express the common markers of the lineage, including CD14, CD16, CD64, CD68, CD71, and CCR5. Exposure to various stimuli can induce M0 macrophages to polarize into several distinct populations identified by surface marker and cytokine/chemokine secretion. Under classical conditions of activation, M0 macrophages are exposed to pro-inflammatory signals such as lipopolysaccharide (LPS), IFNγ, and GM-CSF and polarize into “M1” macrophages that express CD86, CD80, MHC II, IL-1R, TLR2, TLR4, iNOS, and SOCS3 and secrete large amounts of IL-1β, TNFs, IL-12, IL-18, and IL-23. M1 macrophages are associated with pro-inflammatory immune responses such as Th1 and Th17 T cell responses. Exposure to other stimuli polarize macrophages into a diverse group of “alternatively activated” or “M2” type cells, which are subdivided into M2a, M2b, M2c, and M2d based on phenotype. M2a is induced by IL-4, IL-13, and fungal infections. M2b is induced by IL-1R ligands, immune complexes, and LPS. M2c polarization occurs in response to IL-10 and TGFβ, and M2d occurs in response to IL-6 and adenosine. Unlike M1 macrophages, M2 cells secrete cytokines such as IL-10 and TGFβ that induce Th2 T cell responses, and are less able to act as antigen presenting cells; functions typically associated with immune regulation and suppression in the tumor microenvironment. In general, M1 macrophages are inflammatory in nature, while M2 macrophages are anti-inflammatory. Unlike other immune cells, whose differentiation is usually permanent, polarized macrophages have been observed to undergo “reprogramming” from M2 to M1 phenotypes based on pro-inflammatory signaling changes in their immediate environment. This plasticity in macrophage function forms the basis of therapeutic strategies to redirect macrophages to become more cytotoxic.

### Macrophages and the Tumor Microenvironment

Avoiding detection by the immune system is a key factor in the development and growth of a tumor. As such, tumors have evolved to take advantage of numerous overlapping mechanisms of immune regulation, including suppressive immune cells like regulatory T cells and myeloid-derived suppressor cells and creating microenvironments lacking in nutrients critical to cytotoxic T cell function. Just as cancer is a heterogeneous disease, tumors can have varying levels of immune suppression that affect prognosis and the potential effectiveness of immunotherapies. So-called “cold” tumors are characterized by high levels of regulation and a lack of CD8+ T cell infiltration and function. As such, a “cold” tumor microenvironment is associated with a more aggressive disease and poorer treatment outcomes. In contrast a “hot” tumor possesses a more inflammatory immune environment that favors CD8+ T cell infiltration and cytotoxicity. Treatment strategies that would “warm up” the signaling environment of a tumor from “cold” to “hot” would greatly optimize immunotherapy efficacy.

Accumulating evidence suggests that macrophages are abundant in the tumor microenvironment of numerous cancers where they can adopt any of several phenotypes that do not neatly fit into traditional M1/M2 categories and are collectively referred to as tumor-associated macrophages (TAMs). The immunosuppressive nature of the tumor microenvironment typically results in more M2-like TAMs, which further contribute to the general suppression of anti-tumor immune responses. Recent studies, however, have identified that TAMs are able to be “reprogrammed” via pro-inflammatory signals, and that the switch from M2 to a more M1 phenotype is associated with productive anti-tumor immune responses. Inducing endogenous TAMs to switch to M1-type cells and engineering macrophages that cannot be subverted into M2 would greatly enhance anti-tumor immunotherapy and therefore represents a significant advance in the field, various embodiments of which are provided herein in.

### Dendritic Cells

Dendritic cells (DCs) are bone marrow-derived cells that function as professional antigen presenting cells. Immature DCs are characterized by a high capacity for antigen capture and processing, but low T cell stimulatory capability. Inflammatory mediators promote DC maturation. Once DCs have reached the mature stage, they have undergone a dramatic change in their properties. Specifically, they have substantially lost the ability to capture antigen and have acquired an increased capacity to stimulate T cells. Typically, mature DCs present antigen that has been captured at the level of peripheral tissues to naïve T cells. The ability to genetically engineer DCs with chimeric antigen receptors can, in some embodiments, allow mature DCs to simultaneously have the ability to capture and process antigens and to stimulate T cells.

### Monocytes

Monocytes are multipotent cells that circulate in the blood, bone marrow, and spleen, and generally do not proliferate when in a steady state. Typically, they comprise chemokine receptors and pathogen recognition receptors that mediate migration from blood to tissues, for example, during an infection. Monocytes can produce inflammatory cytokines and/or take up cells and toxic molecules, and can also differentiate into inflammatory DCs or macrophages. In some embodiments, a monocyte expressing a chimeric antigen receptor can differentiate into a macrophage expressing a chimeric antigen receptor. In some embodiments, a monocyte expressing a chimeric antigen receptor can differentiate into a dendritic cell expressing a chimeric antigen receptor. In some embodiments, a monocyte expressing a chimeric antigen receptor can recognize a specific antigen (e.g., via the CAR) and initiate an effector response, including, but not limited to, phagocytosis, induction of apoptosis, cytolysis, release of inflammatory cytokines, and gene expression changes.

### B Cells

Recent evidence also suggests that B cells account for up to 25% of all cells in some tumors and that 40% of tumor-infiltrating lymphocytes in some breast cancer subjects are B cells (Yuen et al. Trends Cancer, 2016, 2(12): 747-757). Additionally, therapeutic immune checkpoint blockade may also target activated B cells, in additional to activated T cells, since PD-1, PD-L1, CTLA-4, and the B7 molecules are expressed on B cells. In addition to the immune-regulatory function of producing antibodies and antibody-antigen complexes, B cells can affect the functions of other immune cells by presenting antigens, providing co-stimulation and secreting cytokines. Membrane-bound immunoglobulin on the B cell surface serves as the cell's receptor for antigen, and is known as a B cell receptor (BCR). Activation of BCRs on the surface of a B cell leads to clonal expansion of that B cell and specific antibody production. Additionally, B cells can internalize an antigen that binds to a BCR and present it to helper (CD4+) T cells. Unlike T cells, B cells can recognize soluble antigen for which their BCR is specific. In some embodiments, a B cell expressing a chimeric antigen receptor can also express BCR. In some embodiments, a B cell can express a chimeric antigen receptor and not express BCR.

### Stem Cells

Stem cells are cells that can renew themselves (self-renewal) and can differentiate to yield some or all of the major specialized cell types of an organ or tissue (multipotency). Hematopoietic stem cells (HSCs) give rise to red and white blood cells and platelets, while mesenchymal stem cells (MSCs) are non-blood stem cells from a variety of tissues. MSCs have been shown to have the ability to differentiate in various cells types such as osteoblasts, chondroblasts, adipocytes, neuroectodermal cells, and hepatocytes. Other types of adult stem cells include mammary, intestinal, endothelial, neural, olfactory, and testicular stem cells. MSCs possess, along with their ability to differentiate into several mesenchymal tissue lineages, the capacity to behave as antigen presenting cells. Once MSCs are stimulated with interferon (IFN)-γ, they can uptake, process and present exogenous antigens through their MHC class II molecules, leading to activation of naïve helper (CD4+) T cells (François et al. Blood, 2009, 114(13): 2632-2638). In some embodiments, the antigen presenting capabilities of a HSC are increased when the HSC expresses a chimeric antigen receptor.

### Chimeric Antigen Receptor (CAR)

In one aspect of the invention, a modified primary cell, for example, a macrophage, dendritic cell, monocyte or B cell, is generated by expressing a CAR therein. Thus, the present invention encompasses provided CARs, and a nucleic acid construct encoding provided CARs, wherein the CAR includes an antigen binding domain, a transmembrane domain and an intracellular domain.

In one aspect, the invention includes a cell including a chimeric antigen receptor (CAR), wherein the CAR comprises an antigen binding domain, a transmembrane domain and an intracellular domain, wherein the cell is a primary cell, a macrophage, a dendritic cell, a monocyte or a B cell that expresses the CAR. In some embodiments, a CAR may further comprise one or more of a linker/spacer domain, a co-stimulatory domain, and a destabilizing domain. In some embodiments a cell (e.g. an antigen presenting cell) expressing a CAR may comprise one or more control systems including, but not limited to: a safety switch (e.g., an on switch, and off switch, a suicide switch), a logic gate, for example an AND gate (e.g., two or more CARs, each of which lacks one or more signaling domains such that activation of both/all CARs is required for full T-cell activation or function), an OR gate (e.g., two or more CARs, each with an intracellular domain such as CD3ζ and a co-stimulatory domain), and/or a NOT gate (e.g., two or more CARs, one of which includes an inhibitory domain that antagonizes the function of the other CAR[s]).

In another aspect, the present invention provides a cell including a nucleic acid sequence (e.g., an isolated nucleic acid sequence) encoding a chimeric antigen receptor (CAR), wherein the nucleic acid sequence comprises a nucleic acid sequence encoding an antigen binding domain, a nucleic acid sequence encoding a transmembrane domain and a nucleic acid sequence encoding an intracellular domain, wherein the cell is a monocyte, macrophage and/or a dendritic cell that expresses the CAR. In some embodiments, a single nucleic acid sequence may encode at least two of an antigen binding domain, a transmembrane domain, and an intracellular domain.

In one aspect, the invention includes a modified cell comprising a chimeric antigen receptor (CAR), wherein the CAR comprises an antigen binding domain, a transmembrane domain and an intracellular domain of a co-stimulatory molecule, and wherein the cell is a primary cell, a macrophage, a dendritic cell, a monocyte or a B cell that possesses targeted effector activity. In another aspect, the invention includes a modified cell comprising a nucleic acid sequence encoding a chimeric antigen receptor (CAR), wherein the nucleic acid sequence comprises a nucleic acid sequence encoding an antigen binding domain, a nucleic acid sequence encoding a transmembrane domain and a nucleic acid sequence encoding an intracellular domain of a co-stimulatory molecule, and wherein the cell is a primary cell, a macrophage, a dendritic cell, a monocyte or a B cell that expresses the CAR and possesses targeted effector activity. In some embodiments, targeted effector activity is directed against an antigen on a target cell that specifically binds the antigen binding domain of the CAR. In some embodiments, targeted effector activity is selected from the group consisting of phagocytosis, targeted cellular cytotoxicity, antigen presentation, and cytokine secretion.

### Antigen Binding Domain

In some embodiments, a CAR of the invention comprises an antigen binding domain that binds to an antigen on a target cell. Examples of cell surface markers that may act as an antigen that binds to the antigen binding domain of the CAR include those associated with viral, bacterial and parasitic infections, autoimmune disease, and cancer cells.

The choice of antigen binding domain depends upon the type and number of antigens that are present on the surface of a target cell. For example, the antigen binding domain may be chosen to recognize an antigen that acts as a cell surface marker on a target cell associated with a particular disease state.

In some embodiments, the antigen binding domain binds to a tumor antigen, such as an antigen that is specific for a tumor or cancer of interest. In some embodiments the tumor antigen of the present invention comprises one or more antigenic cancer epitopes. Nonlimiting examples of tumor associated antigens include CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LTLRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

The antigen binding domain can include any domain that binds to an antigen and may include, but is not limited to, a monoclonal antibody, a polyclonal antibody, a synthetic antibody, a human antibody, a humanized antibody, a non-human antibody, and any fragment thereof, for example a scFv. In addition, in some embodiments, an antigen binding domain can be or include an aptamer, a darpin, a centyrin, a naturally occurring or synthetic receptor, affibodies, or other engineered protein recognition molecule. In some embodiments, the antigen binding domain portion comprises a mammalian antibody or a fragment thereof. In some embodiments, the antigen binding domain of the CAR is selected from the group consisting of an anti-CD19 antibody, an anti-HER2 antibody, an anti-mesothelin antibody, or any fragment thereof.

In some instances, the antigen binding domain is derived, in whole or in part, from the same species in which the CAR will ultimately be used in. For example, for use in humans, an antigen binding domain of the CAR comprises a human antibody, a humanized antibody, or a fragment thereof (e.g. a scFV).

In some aspects of the invention, an antigen binding domain is operably linked to another domain of the CAR, such as the transmembrane domain or the intracellular domain, for expression in the cell. In some embodiments, a nucleic acid encoding the antigen binding domain is operably linked to a nucleic acid encoding a transmembrane domain and the transmembrane domain is operably linked to a nucleic acid encoding an intracellular domain.

In some embodiments, a modified cell (e.g., a modified primary cell, monocyte, macrophage, dendritic cell, or B cell) comprising a CAR further comprises one or more additional antigen-binding domain(s) that is required for activation (e.g., a bispecific CAR or bispecific modified cell). In some embodiments, a bispecific modified cell can reduce off-target and/or on-target off-tissue effects by requiring that two antigens are present. In some embodiments, a CAR and an additional antigen-binding domain provide distinct signals that in isolation are insufficient to mediate activation of the modified cell, but are synergistic together, stimulating activation of the modified cell. In some embodiments, such a construct may be referred to as an ‘AND’ logic gate.

In some embodiments, a bispecific modified cell can reduce off-target and/or on-target off-tissue effects by requiring that one antigen is present and a second, normal protein antigen is absent before the cell's activity is stimulated. In some embodiments, such a construct may be referred to as a ‘NOT’ logic gate. In contrast to AND gates, NOT gated CAR-modified cells are activated by binding to a single antigen. However, the binding of a second receptor to the second antigen functions to override the activating signal being perpetuated through the CAR. Typically, such an inhibitory receptor would be targeted against an antigen that is abundantly expressed in a normal tissue but is absent in tumor tissue.

### Transmembrane Domain

In some embodiments, a CAR can be designed to comprise a transmembrane domain that connects the antigen binding domain of the CAR to the intracellular domain. In some embodiments, a transmembrane domain is naturally associated with one or more of the domains in the CAR. In some instances, a transmembrane domain can be selected or modified by amino acid substitution to avoid binding of such domains to the transmembrane domains of the same or different surface membrane proteins to minimize interactions with other members of the receptor complex.

In some embodiments, a transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9. In some embodiments, a transmembrane region may comprise one or more hinge regions. In some instances, any of a variety of human hinge regions can be employed as well (e.g., a CD28 or CD8 hinge region) including the human Ig (immunoglobulin) hinge region.

In some embodiments, a transmembrane domain may be synthetic, in which case it will comprise predominantly hydrophobic residues such as leucine and valine. In some embodiments, a triplet of phenylalanine, tryptophan and valine will be found at each end of a synthetic transmembrane domain.

### Intracellular Domain

In some embodiments, an intracellular domain and/or other cytoplasmic domain of a CAR, includes a similar or the same intracellular domain as the chimeric intracellular signaling molecule described elsewhere herein, and is responsible for activation of the cell in which the CAR is expressed.

In some embodiments, an intracellular domain of a CAR includes at least one domain responsible for signal activation and/or transduction. In some embodiments, an intracellular domain is or comprises at least one of a co-stimulatory molecule and a signaling domain. In some embodiments, an intracellular domain of the CAR comprises dual signaling domains. In some embodiments, an intracellular domain of the CAR comprises more than two signaling domains.

Examples of an intracellular domain for use in some embodiments of the invention include, but are not limited to, the cytoplasmic portion of a surface receptor, co-stimulatory molecule, and any molecule that acts in concert to initiate signal transduction in a primary cell (e.g., a macrophage, dendritic cell, monocyte or B cell), as well as any derivative or variant of these elements and any synthetic sequence that has the same functional capability.

Examples of an intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

In some embodiments, an intracellular domain of a CAR comprises dual signaling domains, such as 41BB, CD28, ICOS, TLR1, TLR2, TLR3, TLR4, TLR5, TLR6, TLR7, TLR8, TLR9, TLR10, TLR11, CD116 receptor beta chain, CSF1-R, LRP1/CD91, SR-A1, SR-A2, MARCO, SR-CL1, SR-CL2, SR-C, SR-E, CR1, CR3, CR4, dectin 1, DEC-205, DC-SIGN, CD14, CD36, LOX-1, CD11b, together with any of the signaling domains listed in the above paragraph in any combination. In some embodiments, an intracellular domain of a CAR includes any portion of one or more co-stimulatory molecules, such as at least one signaling domain from CD3, Fc epsilon RI gamma chain, any derivative or variant thereof, any synthetic sequence thereof that has the same functional capability, and any combination thereof.

In some embodiments, between an antigen binding domain and a transmembrane domain of a CAR, and/or between the intracellular domain and a transmembrane domain of a CAR, a spacer domain may be incorporated. As used herein, the term “spacer domain” generally means any oligo- or polypeptide that functions to link a transmembrane domain to, either an antigen binding domain or, an intracellular domain in a polypeptide chain. In one embodiment, a spacer domain may comprise up to 300 amino acids, preferably 10 to 100 amino acids and most preferably 25 to 50 amino acids. In another embodiment, a short oligo- or polypeptide linker, preferably between 2 and 10 amino acids in length may form the linkage between a transmembrane domain and an intracellular domain of a CAR. An example of a linker includes a glycine-serine doublet.

### Human Antibodies

In some embodiments, it may be preferable to use human antibodies or fragments thereof in an antigen binding domain of a CAR. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, human heavy and light chain immunoglobulin gene complexes may be introduced randomly or by homologous recombination into mouse embryonic stem cells. Alternatively, a human variable region, constant region, and diversity region may be introduced into mouse embryonic stem cells in addition to the human heavy and light chain genes. Mouse heavy and light chain immunoglobulin genes may be rendered non-functional separately or simultaneously with the introduction of human immunoglobulin loci by homologous recombination. For example, it has been described that the homozygous deletion of an antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Antibodies directed against the target of choice can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For an overview of this technology for producing human antibodies, see, Lonberg and Huszar (Int. Rev. Immunol., 13:65-93 (1995)). For a detailed discussion of this technology for producing human antibodies and human monoclonal antibodies and protocols for producing such antibodies, see, e.g., PCT Publication Nos. WO 98/24893, WO 96/34096, and WO 96/33735; and U.S. Pat. Nos. 5,413,923; 5,625,126; 5,633,425; 5,569,825; 5,661,016; 5,545,806; 5,814,318; and 5,939,598, each of which is incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above. For a specific discussion of transfer of a human germ-line immunoglobulin gene array in germ-line mutant mice that will result in the production of human antibodies upon antigen challenge see, e.g., Jakobovits et al., Proc. Natl. Acad. Sci. USA, 90:2551 (1993); Jakobovits et al., Nature, 362:255-258 (1993); Bruggermann et al., Year in Immunol., 7:33 (1993); and Duchosal et al., Nature, 355:258 (1992).

Human antibodies can also be derived from phage-display libraries (Hoogenboom et al., J. Mol. Biol., 227:381 (1991); Marks et al., J. Mol. Biol., 222:581-597 (1991); Vaughan et al., Nature Biotech., 14:309 (1996)). Phage display technology (McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of unimmunized mice. A repertoire of V genes from unimmunized human donors can be constructed and antibodies to a diverse array of antigens (including self-antigens) can be isolated essentially following the techniques described by Marks et al., J. Mol. Biol., 222:581-597 (1991), or Griffith et al., EMBO J., 12:725-734 (1993). See, also, U.S. Pat. Nos. 5,565,332 and 5,573,905, each of which is incorporated herein by reference in its entirety.

Human antibodies may also be generated by in vitro activated B cells (see, U.S. Pat. Nos. 5,567,610 and 5,229,275, each of which is incorporated herein by reference in its entirety). Human antibodies may also be generated in vitro using hybridoma techniques such as, but not limited to, that described by Roder et al. (Methods Enzymol., 121:140-167 (1986)).

### Humanized Antibodies

Alternatively, in some embodiments, a non-human antibody can be humanized, where specific sequences or regions of the antibody are modified to increase similarity to an antibody naturally produced in a human. For instance, in some embodiments of the present invention, an antibody or fragment thereof may comprise a non-human mammalian scFv. In some embodiments, an antigen binding domain portion is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, preferably improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some framework (FR) residues are substituted by residues from analogous sites in rodent antibodies. Humanization of antibodies can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized that retain high affinity for the target antigen and that possess other favorable biological properties. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A humanized antibody retains a similar antigenic specificity as the original antibody. However, using certain methods of humanization, the affinity and/or specificity of binding of the antibody to the target antigen may be increased using methods of “directed evolution,” as described by Wu et al., J. Mol. Biol., 294:151 (1999), the contents of which are incorporated herein by reference herein in their entirety.

### Vectors

In some embodiments, a vector may be used to introduce a CAR into a cell (e.g., a primary cell, monocyte, macrophage, B cell or dendritic cell) as described elsewhere herein. In one aspect, the invention includes a vector comprising a nucleic acid sequence encoding a CAR as described herein. In some embodiments, a vector comprises a plasmid vector, viral vector, retrotransposon (e.g. piggyback, sleeping beauty), site directed insertion vector (e.g. CRISPR, Zn finger nucleases, TALEN), or suicide expression vector, or other known vector in the art. In some embodiments, introducing a nucleic acid sequence into a cell comprises adenoviral transduction. In some embodiments, adenoviral transduction comprises use of an Ad5f35 adenovirus vector. In some embodiments, an Ad5f35 adenovirus vector is a helper-dependent Ad5F35 adenovirus vector. In some embodiments, an AD5f35 adenovirus vector is an integrating, CD46-targeted, helper-dependent adenovirus HDAd5/35++ vector system.

All constructs mentioned above are capable of use with 3rd generation lentiviral vector plasmids, other viral vectors, or RNA approved for use in human cells. In some embodiments, a vector is a viral vector, such as a lentiviral vector. In some embodiments, a vector is a RNA vector.

The production of any of the molecules described herein can be verified by sequencing. Expression of the full length proteins may be verified using immunoblot, immunohistochemistry, flow cytometry or other technology well known and available in the art.

The present invention, in some embodiments, also provides vectors in which DNA of the present invention is inserted. Vectors, including those derived from retroviruses such as lentivirus, are suitable tools to achieve long-term gene transfer since they allow long-term, stable integration of a transgene and its propagation in daughter cells. Lentiviral vectors have the added advantage over vectors derived from onco-retroviruses, such as murine leukemia viruses, in that they can transduce non-proliferating cells, such as hepatocytes. They also have the added advantage of resulting in low immunogenicity in the subject into which they are introduced.

The expression of natural or synthetic nucleic acids is typically achieved by operably linking a nucleic acid or portions thereof to a promoter, and incorporating the construct into an expression vector. In some embodiments, a vector is one generally capable of replication in a mammalian cell, and/or also capable of integration into the cellular genome of the mammal. Typical vectors contain transcription and translation terminators, initiation sequences, and promoters useful for regulation of the expression of the desired nucleic acid sequence.

A nucleic acid can be cloned into any number of different types of vectors. For example, a nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

An expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193, the contents of which are incorporated herein by reference in their entireties).

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the elongation factor-1α promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

In some embodiments, reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Introduction of Nucleic Acids

In some embodiments, the invention includes methods for modifying a cell comprising introducing (e.g., via transformation or transduction) a nucleic acid sequence (e.g., an exogenous nucleic acid sequence) encoding some or all of a chimeric antigen receptor (CAR) into a cell (e.g., a primary cell, monocyte, macrophage, B cell or dendritic cell), wherein the CAR comprises an antigen binding domain, a transmembrane domain and an intracellular domain, and wherein the cell expresses the CAR. In some embodiments, introducing a CAR into a cell comprises introducing a nucleic acid sequence encoding the CAR. In another embodiment, introducing a nucleic acid sequence comprises electroporating a mRNA encoding the CAR. In some embodiments, the invention includes methods for modifying a cell comprising introducing a nucleic acid sequence (e.g., an isolated or non-native nucleic acid sequence) encoding a chimeric antigen receptor (CAR) into a primary cell, monocyte, macrophage, B cell or dendritic cell, wherein the isolated nucleic acid sequence comprises a nucleic acid sequence encoding an antigen binding domain, a nucleic acid sequence encoding a transmembrane domain and a nucleic acid sequence encoding an intracellular domain, wherein the cell is a primary cell, monocyte, macrophage, B cell or dendritic cell that expresses the CAR. In some embodiments, one or more of the antigen binding domain, transmembrane domain, and the intracellular domain are encoded by separate nucleic acid molecules.

In some embodiments, the invention includes methods for modifying a cell comprising introducing a chimeric antigen receptor (CAR) into the cell, wherein the CAR comprises an antigen binding domain, a transmembrane domain and an intracellular domain, and wherein the cell is a primary cell (e.g., monocyte, macrophage, B cell or dendritic cell) that expresses the CAR. In some embodiments, introducing a CAR into a cell comprises introducing a nucleic acid sequence encoding the CAR (e.g., some components or all of the CAR). In some embodiments, introducing a nucleic acid sequence comprises electroporating DNA or a mRNA encoding the CAR into a cell.

Methods of introducing and expressing genes, such as those that encode a CAR, into a cell are known in the art. In the context of an expression vector, the vector can be readily introduced into a host cell, e.g., mammalian, bacterial, yeast, or insect cell by any method in the art. For example, in some embodiments, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). Nucleic acids can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). Nucleic acids can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

In some embodiments, biological methods for introducing a polynucleotide of interest into a host cell include the use of DNA and RNA vectors. RNA vectors include vectors having a RNA promoter and/or other relevant domains for production of a RNA transcript. Viral vectors, and especially retroviral vectors, have become the most widely used method for inserting genes into mammalian, e.g., human cells. Other viral vectors may be derived from lentivirus, poxviruses, herpes simplex virus, adenoviruses (e.g. Adf535) and adeno-associated viruses, and the like. See, for example, U.S. Pat. Nos. 5,350,674 and 5,585,362.

In some embodiments, introducing a nucleic acid sequence into a cell comprises adenoviral transduction. In some embodiments, adenoviral transduction comprises use of an Ad5f35 adenovirus vector. In some embodiments, an Ad5f35 adenovirus vector is a helper-dependent Ad5F35 adenovirus vector. In some embodiments, an AD5f35 adenovirus vector is an integrating, CD46-targeted, helper-dependent adenovirus HDAd5/35++ vector system.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized (e.g., for introduction of an exogenous nucleic acid in to a cell), an exemplary delivery vehicle is a liposome. The use of lipid formulations is contemplated for the introduction of the nucleic acids into a host cell (in vitro, ex vivo or in vivo). In another aspect, a nucleic acid may be associated with a lipid. A nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the molecules described herein, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In some embodiments, one or more of nucleic acid sequences are introduced by a method selected from the group consisting of transducing the population of cells, transfecting the population of cells, and electroporating the population of cells. In some embodiments, a population of cells comprises one or more of the nucleic acid sequences described herein. In some embodiments, one or more nucleic acids are transfected, transduced and/or electroporated with one or more nuclease enzymes (e.g. Cas9 or Cas12a, for example).

In some embodiments, nucleic acids introduced into a cell are or comprise RNA. In some embodiments, RNA is or comprises mRNA that comprises in vitro transcribed RNA or synthetic RNA. In some embodiments, RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In some embodiments, a desired template for in vitro transcription is a CAR.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. Primers can be designed to be substantially complementary to any portion of the DNA template. For example, the primers can be designed to amplify the portion of a gene that is normally transcribed in cells (the open reading frame), including 5′ and 3′ UTRs. Primers can also be designed to amplify a portion of a gene that encodes a particular domain of interest. In one embodiment, primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In some embodiments, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, a 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, a promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, a mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100 T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

Some in vitro-transcribed RNA (IVT-RNA) vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In some embodiments, a RNA construct is delivered into cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Sources of Cells

In some embodiments, phagocytic cells are used in the compositions and methods described herein. In some embodiments, a source of phagocytic cells, such as primary cell, monocyte, macrophage, B cell or dendritic cell, is obtained from a subject. In some embodiments, one or more stem cells may be used to provide desired antigen presenting cells (e.g., monocyte, macrophage, B cell or dendritic cell). Non-limiting examples of subjects include humans, dogs, cats, mice, rats, and transgenic species thereof. Preferably, the subject is a human. Cells can be obtained from a number of sources, including peripheral blood mononuclear cells, bone marrow, lymph node tissue, spleen tissue, umbilical cord, tumors, and induced pluripotent stem cells. In certain embodiments, any number of primary cell, monocyte, macrophage, B cell, dendritic cell or progenitor cell lines available in the art, may be used. In certain embodiments, cells can be obtained from a unit of blood collected from a subject using any number of techniques known to the skilled artisan, such as Ficoll separation. In some embodiments, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. Cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, precursors to primary cells, monocytes, macrophages, B cells and/or dendritic cells may be used (e.g., stem cells). Non-limiting examples include, hematopoietic stem cells, common myeloid progenitors, myeloblasts, monoblasts, promonocytes, and intermediates. In some embodiments, induced pluripotent stem cells may be used as a source of generating primary cells, monocytes, macrophages, B cells and/or dendritic cells. In some embodiments, any cells derived from hematopoietic stem cells that are capable of acting as APCs can be used.

If myeloid precursors are used, such as hematopoietic stem cells, they may be ex vivo differentiated into primary cells, monocytes, macrophages, B cells, and/or dendritic cells, or precursors of said pathway. In addition, precursors (such as but not limited to hematopoietic stem cells) may be used as the therapeutic cell, such that the myeloid differentiation occurs in vivo. Cells may be autologous or sourced from allogeneic or universal donors. In some embodiments, myeloid progenitors or hematopoietic stem cells may be engineered such that expression of the CAR is under the control of a cell type specific promoter, such as a known myeloid, macrophage, monocyte, dendritic cell, microglial cell, M1 specific, or M2 specific promoter.

In some embodiments, monocytes or precursors may be ex vivo differentiated into microglial cells prior to infusion with cytokines known to those in the art. In some embodiments, differentiation of monocytes into microglial cells may improve activity in the central nervous system.

In some embodiments, induced pluripotent stem cells may be derived from normal human tissue, such as peripheral blood, fibroblasts, skin, keratinocytes, renal epithelial cells, or other cells reprogrammed with the genes OCT4, SOX2, KLF4, and C-MYC. In some embodiments, autologous, allogeneic, or universal donor iPSCs could be differentiated toward the myeloid lineage (monocyte, macrophage, dendritic cell, and/or precursor thereof).

In some embodiments, cells are isolated from peripheral blood by lysing the red blood cells and depleting the lymphocytes and red blood cells, for example, by centrifugation through a PERCOLL™ gradient. Alternatively, cells can be isolated from umbilical cord. In any event, a specific subpopulation of the primary cells, monocytes, macrophages, B cells and/or dendritic cells can be further isolated by positive or negative selection techniques.

In some embodiments, mononuclear cells so isolated can be depleted of cells expressing certain antigens, including, but not limited to, CD34, CD3, CD4, CD8, CD14, CD19 or CD20. Depletion of these cells can be accomplished using an isolated antibody, a biological sample comprising an antibody, such as ascites fluid, an antibody bound to a physical support, and a cell bound antibody.

Enrichment of a primary cell e.g., monocyte, macrophage, B cell and/or dendritic cell) population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, enrichment of a cell population for primary cells (e.g., monocytes, macrophages, B cells and/or dendritic cells) by negative selection can be accomplished using a monoclonal antibody cocktail that typically includes antibodies to CD34, CD3, CD4, CD8, CD14, CD19 or CD20.

During isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in some embodiments, a concentration of 2 billion cells/ml is used. In some embodiments, a concentration of 1 billion cells/ml is used. In some embodiments, greater than 100 million cells/ml is used. In some embodiments, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. The use of high concentrations of cells can result in increased cell yield, cell activation, and cell expansion.

In some embodiments, a population of cells comprises primary cells, monocytes, macrophages, B cells or dendritic cells of the present invention. Examples of a population of cells include, but are not limited to, peripheral blood mononuclear cells, cord blood cells, a purified population of primary cells, monocytes, macrophages, B cells or dendritic cells, and a cell line. In some embodiments, peripheral blood mononuclear cells comprise the population of primary cells, monocytes, macrophages, B cells or dendritic cells. In some embodiments, purified cells comprise a population of primary cells (e.g., monocytes, macrophages, B cells or dendritic cells).

In some embodiments, cells have upregulated M1 markers and/or downregulated M2 markers. For example, in some embodiments, at least one M1 marker, such as HLA DR, CD86, CD80, and PDL1, is upregulated in the phagocytic cell. In another example, at least one M2 marker, such as CD206, CD163, is downregulated in the phagocytic cell. In one embodiment, the cell has at least one upregulated M1 marker and at least one downregulated M2 marker.

In yet another embodiment, targeted effector activity in a phagocytic cell is enhanced by inhibition of either CD47 or SIRPα activity. CD47 and/or SIRPα activity may be inhibited by treating the phagocytic cell with an anti-CD47 or anti-SIRPα antibody. Alternatively, CD47 or SIRPα activity may be inhibited by any method known to those skilled in the art.

### Expansion of Cells

In one embodiment, cells or population of cells comprising primary cells, monocytes, macrophages, B cells or dendritic cells are cultured for expansion. In another embodiment, cells or population of cells comprising progenitor cells are cultured for differentiation and expansion of primary cells, monocytes, macrophages, B cells or dendritic cells. The present invention comprises, inter alia, expanding a population of primary cells, monocytes, macrophages, B cells or dendritic cells comprising a chimeric antigen receptor as described herein.

In some embodiments, following culturing, provided cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The culture medium may be replaced during the culture of the cells at any time. Preferably, the culture medium is replaced about every 2 to 3 days. The cells are then harvested from the culture apparatus whereupon the cells can be used immediately or stored for use at a later time

The culturing step as described herein (contact with agents as described herein) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

In some embodiments, cells may be cultured for several hours (about 3 hours) to about 14 days or any hourly integer value in between. Conditions appropriate for cell culture include an appropriate media (e.g., macrophage complete medium, DMEM/F12, DMEM/F12-10 (Invitrogen)) that may contain factors necessary for proliferation and viability, including serum (e.g., fetal bovine or human serum), L-glutamine, insulin, M-CSF, GM-CSF, IL-10, IL-12, IL-15, TGF-beta, and TNF-α. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of the cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the cells may include an agent that can activate the cells. For example, an agent that is known in the art to activate primary cells, monocytes, macrophages, B cells or dendritic cells is included in the culture medium.

### Therapy

In some embodiments, modified cells described herein may be included in a composition for treatment of a subject. In one aspect, a provided composition comprises a modified cell comprising a chimeric antigen receptor described herein. In some embodiments, a composition may include a pharmaceutical composition and further include a pharmaceutically acceptable carrier. A therapeutically effective amount of a pharmaceutical composition comprising the modified cells may be administered.

In one aspect, the invention includes methods of treating a disease or disorder or condition in a subject comprising administering to the subject a therapeutically effective amount of a pharmaceutical composition comprising a modified cell described herein. In some embodiments, a disease or disorder or condition is a neurodegenerative disease/disorder, an inflammatory disease/disorder, a cardiovascular disease/disorder, a fibrotic disease/disorder or a disease associated with a tumor or cancer, or cancer, or amyloidosis. In another aspect, the invention includes methods of treating a solid tumor in a subject, comprising administering to the subject a therapeutically effective amount of a pharmaceutical composition comprising the modified cell described herein. In another aspect, the invention includes methods for stimulating an immune response to a target tumor cell or tumor tissue in a subject comprising administering to a subject a therapeutically effective amount of a pharmaceutical composition comprising the modified cell described herein. In yet another aspect, the invention includes use of modified cells as described herein in the manufacture of a medicament for the treatment of an immune response in a subject in need thereof. In still another aspect, the invention includes use of modified cells as described herein in the manufacture of a medicament for the treatment of a tumor, or cancer, or neurodegenerative disease/disorder, or inflammatory disease/disorder, or cardiovascular disease/disorder, or fibrotic disease/disorder, or amyloidosis in a subject in need thereof.

In certain embodiments, modified cells generated as described herein possess targeted effector activity. In some embodiments, modified cells have targeted effector activity directed against an antigen on a target cell, such as through specific binding to an antigen binding domain of a CAR. In some embodiments, targeted effector activity includes, but is not limited to, phagocytosis, targeted cellular cytotoxicity, antigen presentation, and cytokine secretion.

In some embodiments, a modified cell as described herein has the capacity to deliver an agent, a biological agent or a therapeutic agent to a target. A cell may be modified or engineered to deliver an agent to a target, wherein the agent is selected from the group consisting of a nucleic acid, an antibiotic, an anti-inflammatory agent, an antibody or antibody fragments thereof, a growth factor, a cytokine, an enzyme, a protein, a peptide, a fusion protein, a synthetic molecule, an organic molecule, a carbohydrate or the like, a lipid, a hormone, a microsome, a derivative or a variation thereof, and any combination thereof. As a non-limiting example, a macrophage modified with a CAR that targets a tumor antigen is capable of secreting an agent, such as a cytokine or antibody, to aid in macrophage function. Antibodies, such as anti-CD47/antiSIRPα mAB, may also aid in macrophage function. In yet another example, the macrophage modified with a CAR that targets a tumor antigen is engineered to encode a siRNA that aids macrophage function by downregulating inhibitory genes (i.e. SIRPα). Another example, the CAR macrophage is engineered to express a dominant negative (or otherwise mutated) version of a receptor or enzyme that aids in macrophage function.

In some embodiments, a macrophage is modified with multiple genes, wherein at least one gene includes a CAR and at least one other gene comprises a genetic element that enhances CAR macrophage function. In some embodiments, a macrophage is modified with multiple genes, wherein at least one gene includes a CAR and at least one other gene aids or reprograms the function of other immune cells (such as T cells within the tumor microenvironment).

Further, in some embodiments, provided modified cells can be administered to an animal, preferably a mammal, even more preferably a human, to suppress an immune reaction, such as those common to autoimmune diseases such as diabetes, psoriasis, rheumatoid arthritis, multiple sclerosis, GVHD, enhancing allograft tolerance induction, transplant rejection, and the like. In addition, the cells of the present invention can be used for the treatment of any condition in which a diminished or otherwise inhibited immune response, especially a cell-mediated immune response, is desirable to treat or alleviate the disease. In one aspect, the invention includes treating a condition, such as an autoimmune disease, in a subject, comprising administering to the subject a therapeutically effective amount of a pharmaceutical composition comprising a population of the cells described herein. In addition, the cells of the present invention can be administered as pre-treatment or conditioning prior to treatment with an alternative anti-cancer immunotherapy, including but not limited to CAR T cells, tumor-infiltrating lymphocyte, or a checkpoint inhibitor.

Examples of autoimmune disease include but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

In some embodiments, provided cells can also be used to treat inflammatory disorders. Examples of inflammatory disorders include but are not limited to, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

In some embodiments, cells of the present invention can be used to treat cancers. Cancers include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the cells of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyngioma, ependymoma, pinealoma, hemangioblastoma, acoustic neuroma, oligodendroglioma, menangioma, neuroblastoma, retinoblastoma and brain metastases).

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

In some embodiments, cells of the invention to be administered may be autologous, allogeneic or xenogeneic with respect to the subject undergoing therapy.

The administration of the cells of the invention may be carried out in any convenient manner known to those of skill in the art. In some embodiments, cells of the present invention may be administered to a subject by aerosol inhalation, injection, ingestion, transfusion, implantation or transplantation. In some embodiments, compositions described herein may be administered to a patient transarterially, subcutaneously, intradermally, intratumorally, intranodally, intramedullary, intramuscularly, by intravenous (i.v.) injection, or intraperitoneally. In other instances, provided cells are injected directly into a site of inflammation in the subject, a local disease site in the subject, alymph node, an organ, a tumor, and the like.

### Pharmaceutical Compositions

Pharmaceutical compositions of the present invention may comprise cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, compositions of the present invention are preferably formulated for intravenous administration. In some embodiments, the invention includes pharmaceutical compositions comprising a cell which has been transduced according to the method of any one of the above claims, wherein the cell exhibits an increase in the antigen presenting ability of the cell as compared to a cell of the same type not having been so transduced.

Pharmaceutical compositions of the present invention may be administered in a manner appropriate to the disease/disorder/condition to be treated (or prevented). The quantity and frequency of administration will be determined by such factors as the condition of the patient, and the type and severity of the patient's disease/disorder/condition, although appropriate dosages may be determined by clinical trials.

When “an immunologically effective amount”, “an anti-immune response effective amount”, “an immune response-inhibiting effective amount”, or “therapeutic amount” is indicated, the precise amount of the compositions of the present invention to be administered can be determined by a physician with consideration of individual differences in age, weight, immune response, and condition of the patient (subject). It can generally be stated that a pharmaceutical composition comprising the cells described herein may be administered at a dosage of 10⁴ to 10⁹ cells/kg body weight, preferably 10⁵ to 10⁶ cells/kg body weight, including all integer values within those ranges. The cell compositions described herein may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease/disorder/condition and adjusting the treatment accordingly.

In certain embodiments, it may be desired to administer primary cells, monocytes, macrophages, B cells or dendritic cells to a subject and then subsequently redraw blood (or have an apheresis performed), activate the primary cells, monocytes, macrophages, B cells or dendritic cells therefrom according to the present invention, and reinfuse the patient with these activated cells. This process can be carried out multiple times every few weeks. In certain embodiments, cells can be activated from blood draws of from 10 ml to 400 ml. In certain embodiments, cells are activated from blood draws of 20 ml, 30 ml, 40 ml, 50 ml, 60 ml, 70 ml, 80 ml, 90 ml, or 100 ml. Not to be bound by theory, using this multiple blood draw/multiple reinfusion protocol, may select out certain populations of cells.

In certain embodiments of the present invention, cells are modified using the methods described herein, or other methods known in the art where the cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or treatments for PML patients. In further embodiments, the cells of the invention may be used in combination with CART cell therapy, chemotherapy, radiation, immunosuppressive agents, such as cyclosporin, azathioprine, methotrexate, mycophenolate, and FK506, antibodies, or other immunoablative agents such as anti-CD52 antibody alemtuzumab (CAM PATH), anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the cell compositions of the present invention are administered to a patient in conjunction with (e.g., before, simultaneously or following) bone marrow transplantation, lymphocyte ablative therapy using either chemotherapy agents such as, fludarabine, external-beam radiation therapy (XRT), cyclophosphamide, Rituxan, or antibodies such as OKT3 or CAMPATH. For example, in one embodiment, subjects may undergo standard treatment with high dose chemotherapy followed by peripheral blood stem cell transplantation. In certain embodiments, following the transplant, subjects receive an infusion of the cells of the present invention. In an additional embodiment, the cells may be administered before or following surgery.

The dosage of the above treatments to be administered to a subject will vary with the precise nature of the condition being treated and the recipient of the treatment. The scaling of dosages for human administration can be performed according to art-accepted practices. The dose for CAMPATH antibody, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

It should be understood that methods and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in these experiments are now described.

Cell lines: The THP-1, SKOV3, K562, MDA-468, CRL-2351, HTB-20, HTB-85, CRL-5803, CRL-5822, CRL-1555, HTB-131, HTB-20, and CRL-1902 cell lines were purchased from American Type Culture Collection (ATCC). Cells were culture in RPMI media with 10% fetal bovine serum, penicillin, streptomycin, 1× Glutamax, and 1× HEPES unless otherwise recommended by ATCC. All cell lines were transduced with a lentiviral vector co-encoding click beetle green (CBG) luciferase and GFP under an EF1α promoter, separated by a P2A sequence. Transduced target cell lines were FACS sorted for 100% GFP positivity prior to use as targets in vitro and in vivo. THP-1 cells were lentivirally transduced, FACS sorted, and maintained in liquid culture. CAR expression and purity was routinely validated.

Plasmid construction and virus: For lentivirus production, CAR constructs were cloned into the third generation pTRPE lentiviral backbone using standard molecular biology techniques. All CAR constructs utilized a CD8 leader sequence, (GGGGS)₃ (SEQ ID NO: 1) linker, CD8 hinge, and CD8 transmembrane domain and were expressed under the control of an EF1α promoter. Lentivirus was packaged in 293 cells and purified/concentrated as described previously (Gill, S. et al. (2014) Blood 123, 2343-2354). In indicated experiments, Vpx was incorporated into lentivirus at the packaging stage as previously described (Bobadilla, S. et al. (2013) Gene Ther. 20, 514-520). Cell lines were transduced with lentivirus MOI 3-5 unless otherwise noted. For replication deficient adenovirus production, anti-HER2 CAR was cloned into the pShuttle transfer plasmid using Xba-I and Sal-I, then cloned into pAd5f35 using I-Ceu I and PI-Sce I. All cloning steps were validated by restriction enzyme digest and sequencing. pAd5f35 is first generation E1/E3 deleted adenoviral backbone. Ad5f35-CAR-HER2-zeta was generated, expanded, concentrated, and purified using standard techniques in 293 cells. All adenoviral batches were verified negative for replication competent adenovirus and passed sterility and endotoxin analysis. Adenoviral titer was determined using Adeno-X Rapid Titer Kit (Clontech, USA) and validated by functional transgene expression in human macrophages. An MOI of 1000 PFU/cell was used unless otherwise stated.

Animal studies: Schemas of the utilized xenograft models are shown in detail in the first panel of each relevant figure. NOD/SCID Il2rg^{-/-} hIL3-hGMCSF-hSF (NSG-SM3 or NSGS) mice originally obtained from Jackson Laboratories were purchased and bred. Cells (SKOV3 tumor cells, human macrophages, or human T cells) were injected in 200-300 μL PBS for both IP and IV tail vein injections. IV injections of human macrophages were split into consecutive injections to attain the target dose. Bioluminescent imaging was performed at least weekly using an IVIS Spectrum (Perkin Elmer, USA) and analysis was performed using LivingImage v4.3.1 (Caliper LifeSciences). Mice were weighed weekly and were subject to routine veterinary assessment for signs of overt illness. Animals were euthanized at experimental termination or when predetermined IACUC rodent health endpoints were reached.

Flow cytometry: Primary human macrophages were tested for CAR-HER2 expression using a two-step staining protocol: human HER2/ERBB2 Protein-His tag (Sino Biological Inc, 10004-H08H-100) primary stain followed by Human TruStain FcX (Biolegend, 422302) and Anti-His Tag APC (R&D Systems, IC050A) secondary stain. Macrophage purity was tested using the following panel: Anti-CD11b PE (Biolegend, 301306), Anti-CD14 BV711 (Biolegend, 301838), Anti-CD3 FITC (eBioscience, 11-0038-42), Anti-CD19 PE-CY7 (eBioscience, 25-0198-42), Anti-CD66b PerCP-CY5.5 (Biolegend, 305108), Anti-CD56 BV605 (Biolegend, 318334), and Live/Dead Fixable Aqua Dead Cell Stain Kit (ThermoFisher, L34957). The same panel was used for testing the monocyte purity post CD14 MACS selection, prior to seeding for differentiation. M1/M2 markers on primary human macrophages were detected with the following panel: Anti-CD11B PE (Biolegend, 301306), Anti-CD80 BV605 (Biolegend, 305225), Anti-CD86 BV711 (Biolegend, 305440), Anti CD206 BV421 (Biolegend, 321126), Anti CD163 APC-CY7 (Biolegend, 333622), anti HLA-DR BV785 (Biolegend, 307642), Anti-HLA ABC PE/CY7 (Biolegend, 311430) and Live/Dead Fixable Aqua Dead Cell Stain Kit. CD46 expression was detected with Anti-CD46 APC (Biolegend, 352405) and CXADR was detected with Anti-CAR PE (EMD Millipore, FCMAB418PE-I). Appropriate fluorescence matched isotype controls were acquired from Biolegend. Surface HER2 was detected using Anti-Human CD340/HER2 APC (Biolegend, 324408).

For macrophage staining, cells were detached using a Detachin, collected, pre-treated with FcR blocking agent, and stained with a cocktail antibody. If needed, cells were fixed, permeabilized and stained for intracellular markers and transcription factors. Cells were acquired using an Attune cytometer. Five different antibody panels were used to determine macrophage phenotype.

TABLE 1
Surface panel I

| | | Macrophage |
|---|---|---|
| Antibody | Fluorochrome | Phenotype |
| HLA-DR, DP, DQ | FITC | M1 |
| CD80 | BV421 | M1 |
| CD86 | PE/Cy7 | M1 |
| TLR2 (CD282) | PE | M1 |
| CD163 | PerCP/Cy5.5 | M2 |
| CD204 | APC | M2 |
| CD206 | APC/Fire750 | M2 |

TABLE 2
Surface panel II

| | | Macrophage |
|---|---|---|
| Antibody | Fluorochrome | Phenotype |
| CD40 | PE/Cy7 | M1 |
| TLR4 (CD284) | PE | M1 |
| 4-1BBL (CD137L) | APC/Fire750 | M1 |
| PD-L1 (CD274) | BV711 | M1/M2 |
| CD209 (DC-SIGN) | BV421 | M1 |
| SIRPα/β (CD172α/β) | APC | M2 |
| CSF-1R (CD155) | PerCP/Cy5.5 | M2 |
| TGF-β1 (LAP) | FITC | M2 |

TABLE 3
Intracellular panel I

| | | Macrophage |
|---|---|---|
| Antibody | Fluorochrome | Phenotype |
| IL-1β | FITC | M1 |
| TNF-α | PE/Cy7 | M1 |
| INF-α | Alexa Fluor 647 | M1 |
| INF-γ | BV421 | M1 |
| IL-10 | PerCP/Cy5.5 | M2 |
| Arginase I | PE | M2 |

TABLE 4
Intracellular panel II

| | | Macrophage |
|---|---|---|
| Antibody | Fluorochrome | Phenotype |
| CXCL10 | PE | M1 |
| IL-6 | BV421 | M1 |
| IL-8 | PE/Cy7 | M1 |
| IL-12/IL-23 | PerCP/Cy5.5 | M1 |
| IL-15 | APC | M1 |
| IFN-β1 | FITC | M1 |

TABLE 5
Transcription factor panel

| | | Macrophage |
|---|---|---|
| Antibody | Fluorochrome | Phenotype |
| pSTAT1 | FITC | M1 |
| IRF1 | PE | M1 |
| IRF3 | Alexa Fluor 647 | M2 |
| pSTAT3 | PE/Cy7 | M2 |
| pSTAT6 | PerCP/eFluor710 | M2 |

Two antibody panels were used to determine dendritic cell phenotype upon treatment with conditioned media from UTD or CAR macrophages, in addition to staining with CD11c-APC, CD14-PerCP-Cy5.5 and CD11b-PE

TABLE 6
Dendritic cell panel I

| Antibody | Fluorochrome |
|---|---|
| HLA-DR, DP, DQ | FITC |
| CD80 | BV421 |
| CD86 | PE/Cy7 |
| TLR2 (CD282) | PE |
| CD14 | PerCP-Cy5.5 |
| CD1a | APC |
| CD206 | APC/Fire750 |
| CD1c | BV711 |

TABLE 7
Dendritic cell panel II

| Antibody | Fluorochrome |
|---|---|
| CD54 | FITC |
| CD209 (DC-SIGN) | BV421 |
| CD40 | PE/Cy7 |
| TLR4 (CD137L) | PE |
| CD58 | PerCP-Cy5.5 |
| SIRPα/beta (CD172α/β) | APC |
| 4-1BBL (CD137L) | APC/Fire750 |
| CD83 | BV711 |

TruStain FcX (Biolegend, 422302) was always used for FACS staining of monocytes, macrophages, dendritic cells, or monocytic cell lines expressing Fc receptors. Flow cytometry data were acquired on a BD Fortessa with HTS (BD Biosciences, USA), and analyzed with FlowJo X10 (FlowJo, LLC).

FACS based phagocytosis assay: 1×10⁵ UTD or CAR-HER2-zeta human monocyte derived macrophages (48 hours post transduction) were co-cultured with media (Mac Alone), 1×10⁵ GFP+ MDA-468 cells (HER2−) or 1×105 GFP+ SKOV3 (HER2+) target cells for 3-4 hours at 37° C. in triplicate. Following co-culture, cells were harvested with Accutase (Innovate Cell Technologies, Inc., USA) and stained with Anti-CD11b APC-CY7 (Biolegend, 301342) and analyzed via FACS using a BD Fortessa (Beckton Dickinson, New Jersey). The percent of GFP+ events within the CD11b+ population was plotted as percentage phagocytosis. Data are represented as mean+/−standard error of triplicate wells. Statistical significance between CAR-HER2-zeta and UTD was calculated using ANOVA with multiple comparisons; ****p<0.0001, ns=non-significant.

Primary human macrophages and T cells: Normal donor apheresis was either performed at the hematology unit at the Hospital of the University of Pennsylvania under an IRB approved protocol through the Human Immunology Core of the University of Pennsylvania or were acquired and shipped fresh from HemaCare (HemaCare Corporation, CA, USA). Apheresis derived leukopacs were subject to elutriation using an Elutra Cell Separation System (Terumo BCT) to reduce erythrocytes, platelets, lymphocytes, and granulocytes. Monocyte enriched fractions were pooled and subjected to MACS CD14 positive selection (Miltenyi) per manufacturer's instruction. The pre-selection and post-selection (positive and negative fraction) purity was tested using flow cytometry. Selected CD14 monocytes were seeded in Cell Differentiation Bags (Miltenyi) in RPMI with 10% FBS, penicillin, streptomycin, 1× glutamax, 1× HEPES, and 10 ng/mL recombinant human GM-CSF (Peprotech, 300-03) for 7 days. Differentiation was monitored by light microscopy. Adenovirus was added on day 5 at an MOI of 1×103 based on PFU titer. Differentiated macrophages were harvested at day 7 and tested for CAR expression, differentiation, and macrophage purity by FACS. For smaller scale experiments macrophages were plated directly in tissue-culture treated well-plates or flasks and transduced at an MOI of 1000 PFU directly in well plates or flasks. CD3 selected T cells were expanded/transduced as previously described (Gill, S. et al. (2014) Blood 123, 2343-2354).

Microscopy based phagocytosis assay: Control or CAR expressing mRFP+ THP1 cells were plated at 7.5×10⁴ per well in 48 well plates and differentiated with 1 ng/mL phorbol 12-myristate 13-acetate (PMA) in RPMI with 10% FBS for 48 hours. Following differentiation, PMA was washed out with media and 7.5×10⁴ control or target GFP+ K562 tumor cells were added and co-cultured for 4 hours at 37° C. After 4 hours, tumor cells (non-adherent) were washed out and the plate was imaged for mRFP and GFP fluorescence. The average number of phagocytic events in three random fields of view per well were averaged, in triplicate wells, on a 10× field of view. Cells were imaged using an EVOS FL Auto 2 Imaging System (ThermoFisher Scientific, AMAFD2000). Data represent the mean+/−standard error of triplicate wells. Statistical significance was calculated via t-test.

Live video imaging microscopy: 3.0×10⁵ CAR or control mRFP+ THIP-1 cells were differentiated as above in 6 well plates and co-cultured with 3.0×10⁵ control or target GFP+ K562 cells for 16-24 hours in an incubated 37° C. live imaging chamber and imaged ever 30-120 seconds for mRFP and GFP using the EVOS FL Auto 2 Live Imaging System (ThermoFisher, USA) using the 10× lens.

In vitro cytotoxicity assay: CBG/GFP double positive SKOV3, HTB-20, and CRL-2351 tumor cells were used as targets in luciferase based killing assays by control (UTD) or CAR-HER2-zeta (CAR) macrophages. The effector to target (E:T) ratio was serially titrated from 10:1 down to 1:30 for both effector groups. Bioluminescence was measured using an IVIS Spectrum (Perkin Elmer, USA). Percent specific lysis was calculated based on luciferase signal (total flux) relative to tumor alone, using the following formula.

% Specific Lysis=[(Sample signal−Tumor alone signal)/(Background signal−Tumor alone signal)]×100

Data is shown as mean+/−SEM, with each condition in triplicate. Negative specific lysis values indicate more signal than in the tumor alone wells. Statistical significance was calculated using ANOVA with multiple comparisons; ****p<0.0001; ***p<0.001; **p<0.01; *p<0.05; ns=non-significant.

Image cytometry: Control or CAR mRFP+ THP-1s were differentiated and co-cultured with CD19+GFP+ K562 target cells as described above. After 4 hour co-culture, cells were washed and harvested with trypsin-EDTA and stained with L/D aqua for viability. Imaging cytometry was performed on Amnis ImageStreamX (EMD Millipore, Germany). Cells were gated for mRFP+GFP+ events and the phagocytosis erode algorithm was applied, which identifies GFP signal within an mRFP positive event.

Macrophage polarization: For M1 or classically-activated macrophage polarization, human monocyte derived macrophages were exposed to 20 ng/mL recombinant interferon-gamma (Peprotech, 300-02) and 100 ng/mL lipopolysaccharide (LPS-EK, Invivogen, tlrl-eklps) in RPMI with 10% FBS for 24 hours. For M2 or alternatively activated macrophage polarization, human monocyte derived macrophages were exposed to 20 ng/mL recombinant human IL-4 (Peprotech, 200-04) or IL-13 (Peprotech, 200-13). In some experiments, 48-hour conditioned media from SKOV3 was used (50% diluted in RPMI with 10% FBS) to polarize macrophages toward M2 for 24 hours. In experiments where control or CAR macrophages were challenged with M2 inducing cytokines, cells were treated with cytokine for 24 hours, 48 hours post-viral transduction.

RNA-sequencing of human macrophages: RNA was isolated from human macrophages from matched donors, treated as described in each figure and polarized/challenged as above using Ambion RiboPure RNA purification kit (Thermo Fisher Scientific, AM1924). RNA-seq libraries were generated using TruSeq RNA Library Prep Kit (Illumina, RS-122-2001/2) and validated via BioA prior to sequencing. The libraries were sequenced on 75 bp single-end reads using a NextSeq sequencer (Illumina). Low quality reads were trimmed using Trimmomatic (v0.36) and mapped to human genome (hg38) using STAR (v2.6.0c) with default parameters. Gene count was calculated using featureCounts (v1.6.1). Non-expressed genes with read count <1 in all samples were removed prior to differential expression analysis. DESeq2 with log fold change of 1 and adjusted P-value of 0.05 was used to identify differentially expressed genes.

For genome browser tracks, bam files were first converted into bed files using bedtools (v2.27.1). Normalized bedgraph tracks were generated using makeUCSCfile with 10,000,000 normalization factor (Homer v2) and converted into bigwig format for integrative genomics viewer (IGV) usage.

Reads were mapped to the human genome (hg38) using RUM prior to using DegSeq and EdgeR for differential analysis. Ingenuity Pathway Analysis (Qiagen Bioinformatics) was used to map differentially expressed genes to canonical pathways.

Real-time PCR: RNA was isolated using Ambion RiboPure RNA purification kit (Thermo Fisher Scientific, AM1924) and reverse transcribed using iScript RT Supermix for RT-qPCR (Bio-Rad, 1708841). For q-PCR, template cDNA, primers, Taqman Gene Expression primer/probe, and Taqman Gene Expression Master Mix (Applied Biosystems, 4369016) were used per manufacturer's instructions. The following human primer/probes from Applied Biosystems were used: TNF (Hs00174128_m1), IL12A (Hs01073447_m1), GAPDH (Hs02786624_G1), TAP1 (Hs00388675_m1), CD206 (Hs00267207_m1), CD80 (Hs01045161_m1), and IFNB (Hs01077958_s1).

Phytohemagglutinin T cell proliferation assay: Human T cells were labeled with CellTrace CFSE Cell Proliferation Kit (ThermoFisher, C34554) per manufacturer's protocol. CFSE labeled T cells were cultured alone or at a 1:1 E:T ratio for 5 days with control UTD or transduced CAR-HER2-zeta autologous macrophages in the presence or absence of 0.5% phytohumaggluttinin (PHA-L, Sigma-Aldrich, 11249738001). Proliferation of CD8 T cells was determined by FACS by measuring the % loss of CFSE (CFSE dilution).

NY-ESO-1 antigen processing and presentation assay: Primary human macrophages were transduced with HLA-A201-P2A-NY-ESO1 Vpx lentivirus or not (Ag and No Ag, respectively). 1G4 NY-ESO-1 TCR T cells were generated as previously described and stained with CellTrace Violet Cell Proliferation Kit (ThermoFisher, C34557) per manufacturer's instruction (Rapoport, A. P. et al. (2015) Nat. Med. 21, 914-921). 48 hours post lentiviral transduction, macrophages were transduced with Ad5f35-CAR-HER2-zeta for polarization, or not, for an additional 48 hours prior to the addition of CTV labeled 1G4 anti-NY-ESO1 TCR autologous T cells for 5 days. Proliferation of anti-NYESO1 TCR+ CD8+ T cells was determined by FACS by measuring dilution of CTV.

Mitochondrial respiratory analysis in human macrophages: Mitochondrial function was assessed using an extracellular flux analyzer (Agilent/Seahorse Bioscience). Primary human control or 48-hour transduced CAR macrophages, with or without 24-hour exposure to 20 ng/mL recombinant human IL-4 (Peprotech, 200-04) were seeded at 1×10⁵ cells/well onto XF96 cell culture microplates. To assay mitochondrial function, the medium was replaced with XF assay base medium supplemented with 5.5 mM glucose, 2 mM L-glutamine and 1 mM sodium pyruvate. Prior to use, XF96 assay cartridges were calibrated in accordance with the manufacturer's instructions. During instrument calibration (60 min) the cells were switched to a CO₂-free, 37° C., incubator. Cellular oxygen consumption rates (OCR) and extracellular acidification (ECAR) levels were measured under basal conditions and following treatment with 1.5 μM oligomycin, 1.5 μM fluoro-carbonyl cyanide phenylhydrazone (FCCP) and 40 nM rotenone, with 1 M antimycin A.

In vitro transcription and RNA electroporation: In vitro transcription (IVT) was performed using the mMessage mMachine T7 Ultra Kit (ThermoFisher, AM1345). Briefly, the cDNA for human HER2 was cloned into the pDA vector downstream of a T7 promoter, linearized with PacI, and IVT was performed per manufacturer's instruction. For RNA electroporation, MDA-468 cells were washed twice in PBS and resuspended in Opti-MEM (ThermoFisher, 31985062). Increasing amounts of IVT HER2 mRNA were added (from 0 to 20 ug) prior to electroporation using the BTX ECM 830 Square Wave Electroporation System (Harvard Apparatus) using a single pulse of 300V and 0.7 msec. Cells were incubated at 37 C overnight and HER2 MFI was determined via FACS prior to use.

M2 macrophage polarization: 2×10⁶ untransduced (UTD) macrophages were seeded per well of a 6-well plate in 3 mL of TexMACS media supplemented with 10% fetal bovine serum (FBS). Initially, M0 and M1 macrophages were stimulated with 50 ng/mL GM-CSF and M2 macrophages were stimulated with 100 ng/mL M-CSF for 6 days. On day 3, additional fresh media with appropriate cytokines was added to all macrophage subtypes. On day 6, old media was replaced with fresh media containing: 50 ng/mL GM-CSF (for M0 macrophages); 50 ng/mL GM-CSF, 20 ng/mL LPS, 20 ng/mL IFN-γ, and 20 ng/mL TNF-α (for M1 macrophages); 100 ng/mL M-CSF, 20 ng/mL IL-13, and 20 ng/mL IL-4 (for M2A macrophages); 100 ng/mL M-CSF, 20 ng/mL LPS, and 20 ng/mL IL-1RA (for M2B macrophages); 100 ng/mL M-CSF, 10 ng/mL IL-10, and 10 ng/mL TGF-β1 (for M2C macrophages); and 100 ng/mL M-CSF, 50 ng/m IL-6, and 20 ng/mL LIF (for M2D macrophages). Two days later, media from all macrophage subtypes was removed and conditioned media from UTD, or CAR macrophages was added to cells for next 48 hours.

CAR macrophage generation: 15×10⁶ macrophages were transduced with adenovirus (Ad5f35; 1,000 MOI). 48 hours later, conditioned media from UTD and CAR macrophages was collected, filtered through 0.22 μm bottle filter and aliquoted to polarized macrophages.

Killing assay with M0 and M2 macrophages: M0 and M2 (M2A and M2C) macrophages were prepared as previously described. 10,000 SKOV3-GFP cells (from a high HER2 ovarian cancer cell line) were seeded per well of a 96-well plate with or without untransduced (UTD) and CAR macrophages (30,000 cells) in TexMACS media. To determine the effect of M0 and M2 macrophages on the ability of CAR macrophages to kill tumor cells, SKOV3-GFP cells were mixed with CAR macrophages and each subtype of polarized macrophages (10,000 cells). The killing assay was monitored on an IncuCyte S3 for subsequent 3 days.

Monocyte-Derived Dendritic Cell Differentiation: 2×10⁶ freshly isolated monocytes were seeded per well (6-well plate) in 3 ml of TexMACS media supplemented with 10% FBS and stimulated with 50 ng/ml GM-CSF and 20 ng/ml IL-4 for 9 days, with fresh media addition every third day. To induce maturation, on day 9 media from immature dendritic cells was removed and fresh media containing 50 ng/ml GM-CSF, 20 ng/ml IL-4, and 20 ng/ml TNF-α was added for next 48 hours. Afterwards, media from immature and mature dendritic cells was removed and conditioned media from UTD, or CAR macrophages was added to cells for next 48 hours.

Killing assay with primary human lung tumor explant: Untransduced (UTD) or Ad5f35-CAR-HER2 macrophages (CAR) were used as effector cells in a GFP-based killing assay against SKOV3, a high HER2 ovarian cancer cell line. Tumor cells stably expressed GFP to allow for tracking cell growth over time. The tumor burden was measured at 48 hours post-treatment (by GFP intensity via Incucyte S3 fluorescent microscopy). The macrophage to tumor ratio was 3:1. Primary challenge cell suspension was added—either primary human lung tumor explant single cell suspension, control normal lung single cell suspension, or control peripheral blood mononuclear cell single cell suspension. The ratio of primary single cell suspension challenge cells to macrophage was 1:1. Tumor and normal tissue single cell suspensions were generated using techniques standard in the field. The assay was run with an n of 3.

### Sc-RNA Seq Assay

In vivo: Ten (10) NSG (Nod-scid-gamma) mice were engrafted with 5e⁵ human CD34+ cells to generate a humanized immune system (HIS) mouse model. After engraftment of human leukocytes was confirmed by detection of hCD45+ cells in the peripheral blood (42 days post HSC injection), 2e⁶ SKOV3-CBG-GFP tumor cells were injected subcutaneously into the flank. Tumors were allowed to grow for approximately 19 days. Once tumors were established, mice were treated with either 1e⁷ untransduced control human macrophages (n=4), 1e⁷ anti-HER2 CAR macrophages (n=4), or PBS (n=2). After 5 days, tumors were harvested for scRNA seq analysis.

Tumor harvest: Tumors were excised and processed per standard techniques. Briefly, tumors were kept on ice with RPMI before being minced into small pieces and incubated with digestion medium at 37° C. for 25 minutes. The remaining tissues were further crushed using a syringe plunger. The cell suspension was then filtered through a 70 μm nylon gauze and centrifuged at 450×g for 6 minutes. The supernatant was discarded, and the rest of the cells were resuspended in ACK buffer to lyse red blood cells. Density gradient centrifugation was used on the remaining cells to remove dead cells while enriching live mononuclear cells. The layer of mononuclear cells was collected and the final cell count was measured by both Moxi GO and hemocytometer to be 1e⁶/mL.

Single-cell RNA sequencing: Cells were encapsulated into single cell droplets using 10× Chromium controller and libraries were prepared using Chromium Single Cell V(D)J Reagent Kit v2 according to the official protocol. The libraries were sequenced on an Illumina HiSeq4000 with a geometry of 75 bp paired ends.

Single-cell data processing and analysis: FASTQ files were demultiplexed and generated using Cell Ranger (v2.2). Gene sequences of the chimeric antigen receptor and GFP were added to the GRCh38 genome using function—cellranger mkref. Technical and biological replicates of the same condition (CAR-M treated, UTD-treated, untreated, CAR-M in-vitro and UTD in-vitro) were aggregated together using command—cellranger aggr. Downstream analysis were performed using Seurat v.2.3.4. Cells with fewer than 200 genes or genes that were present in 3 cells or fewer were excluded from downstream analysis. The number of genes (nGene) per cell, and percentage of mitochondria(mito.percent) gene expression level were used to further filter cells. Any cell at the top 3 percent of the nGene distribution or that had 20% or more mito.percentage expression was deemed either as a doublet or an apoptotic cell. These cells were filtered out. The subsequent data was log normalized with a factor of 10000 and scaled with number of UMI and mito.percentage. Highly variable genes were used for principal component analysis, and clusters, defined at 0.6 resolution, were visualized using tSNE plot. A “4D5_scFv” gene was used to identify CAR macrophages and a male specific gene (RPS4Y1) was used to differentiate donor cells from endogenous human immune cells. This was possible because the macrophage donor was male and the human CD34+ HSPC donor was female. This gender mismatch made it possible to identify donor UTD macrophages from the engrafted human immune cells. ERBB2, EPCAM and GFP were used to define the tumor population. Subpopulations from different treatment conditions (e.g., CAR-M in vivo and CAR-M in vitro) were merged into one Seurat object. The top differentially-expressed genes from each cluster were identified using the “roc” test. This test returned a classification power score, which was used to determine the top cluster-driving genes. Only genes that were expressed in more than 25% cells and had at least a 25% difference in expression level were considered. QIAGEN Ingenuity Pathway Analysis (IPA) was used for pathway analysis.

Statistics: Statistical analysis was performed in Prism 6.0 (GraphPad, Inc). Each figure legend denotes the statistical test used. Error bars indicate standard error of the mean unless otherwise indicated. ANOVA multiple comparison p-values were generated using Tukey's multiple comparisons test. All t-tests were two-sided. * indicates p<0.05, ** indicates p<0.01, *** indicates p<0.001, and **** indicates p<0.0001.

The results of the experiments are now described.

### Example 1—CAR-Mediated Redirection of Macrophage Phagocytic Activity

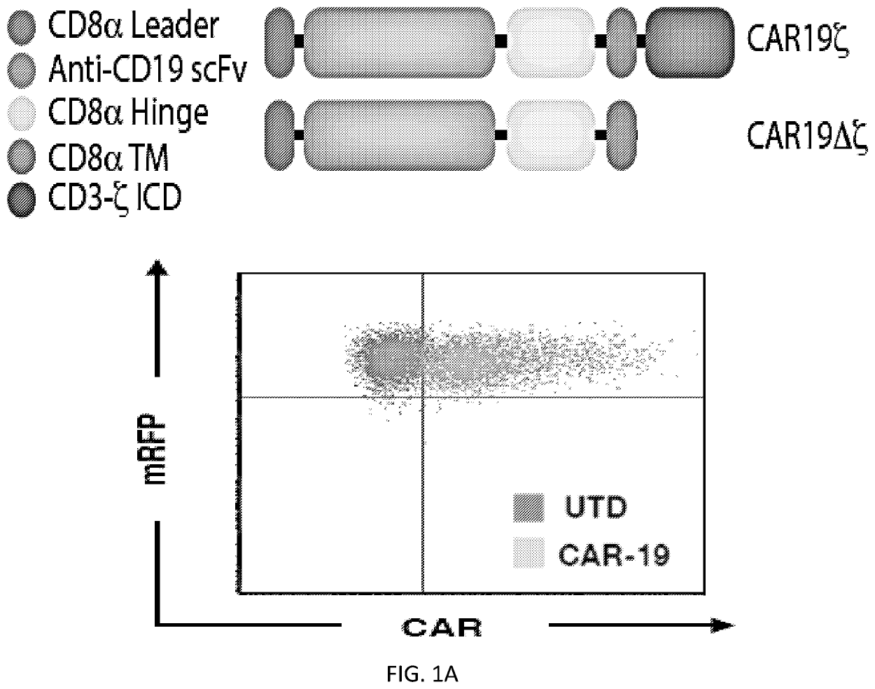

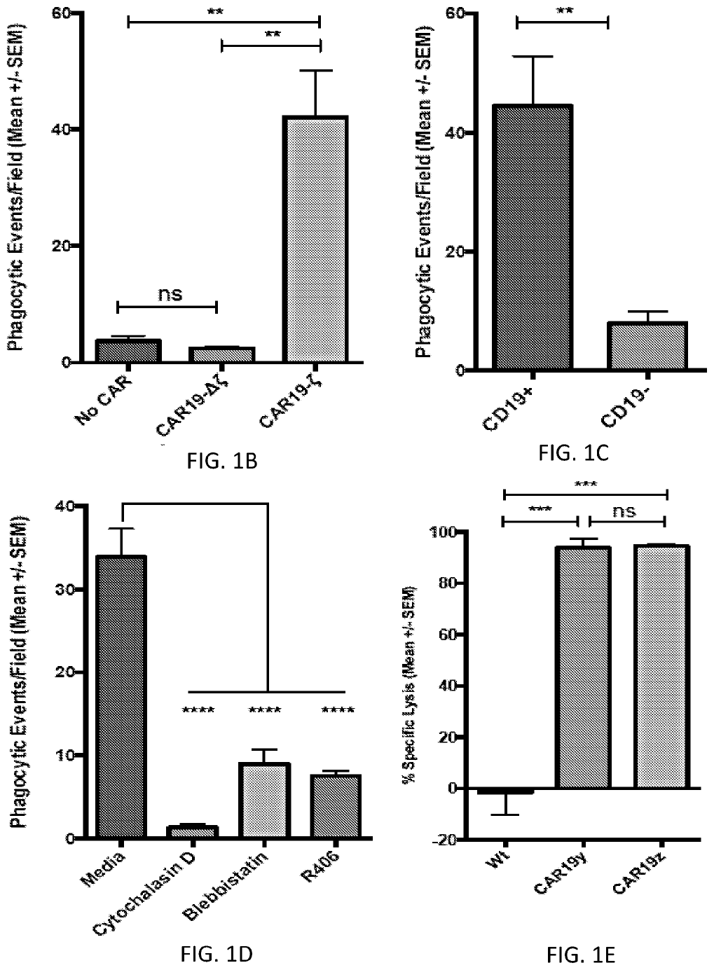

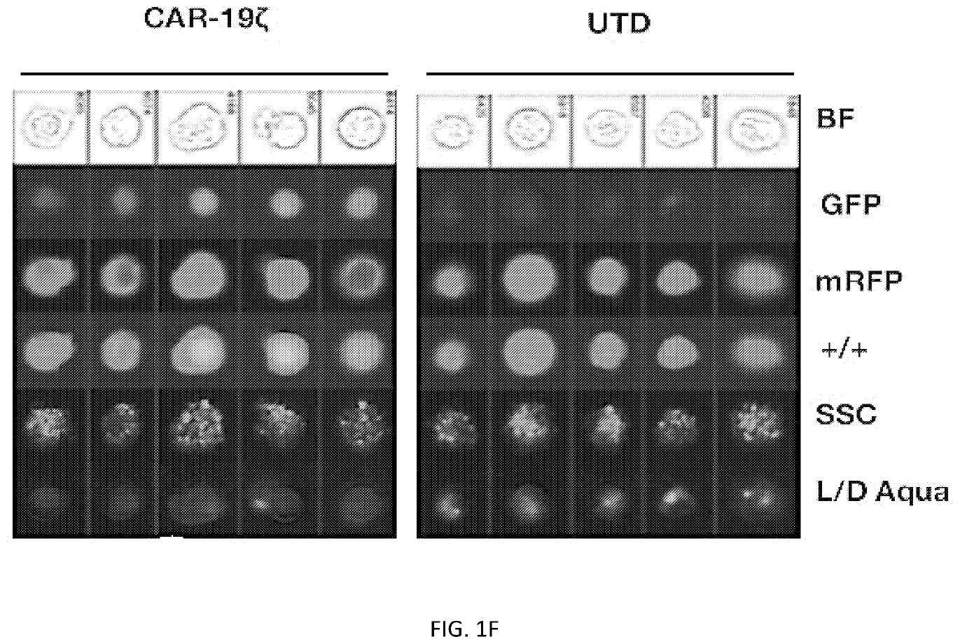

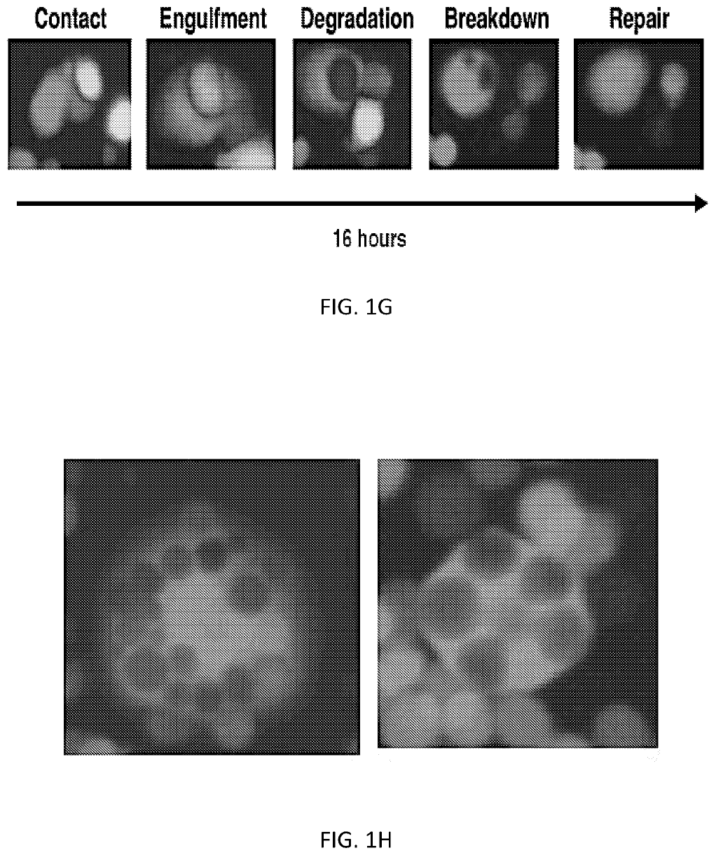

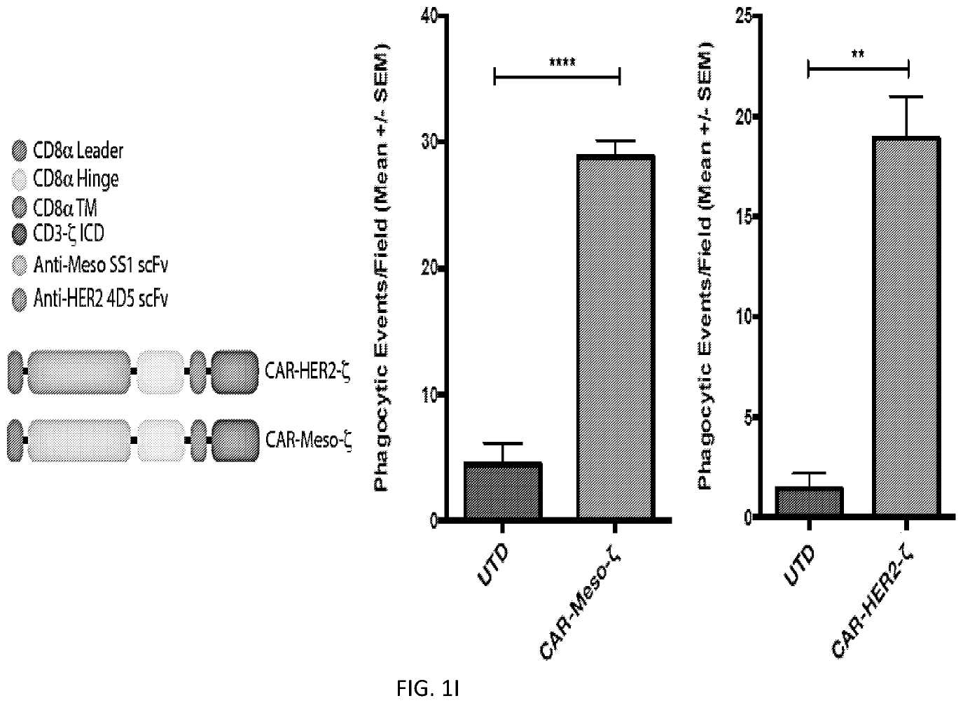

The human macrophage cell line model, THP-1, was first used to test the potential for CAR mediated redirection of macrophage phagocytic activity. The standard CAR expressed in T cells contains the CD3ζ intracellular domain, which bears significant sequence and structural homology to the Fc common gamma chain, FcεRI-γ, which is the canonical signaling molecule for antibody dependent cellular phagocytosis (ADCP) in macrophages. The capacity for CD3ζ-bearing CARs to drive macrophage phagocytosis of antigen bearing tumor cells was tested by expressing an anti-CD19 CAR with ζ signaling (CAR-19ζ) or truncated CAR-19Δζ as a negative control (FIG. 1A). CAR-19ζ but not CAR-19Δζ or control untransduced (UTD) macrophages phagocytosed antigen bearing tumor cells in vitro (FIG. 1B). Furthermore, CAR-19ζ macrophages selectively phagocytosed CD19+ but not CD19− tumor cells (FIG. 1C), demonstrating the need for CAR/antigen binding to drive macrophage phagocytosis. CAR macrophage phagocytosis was an active process requiring Syk, non-muscle myosin IIA, and actin polymerization, similarly to Fc receptor mediated ADCP (FIG. 1D). Anti-CD19 CAR dependent phagocytosis of CD19+ cells was equivalent in macrophages expressing CD3Δζ and Fcγ based CARs (FIG. 1E) and, therefore all subsequent experiments were performed using CD3ζ as the primary CAR intracellular domain. CAR macrophage phagocytosis was confirmed by imaging flow cytometry (FIG. 1F). The behavior of a single CAR macrophage was tracked over time and key steps of the phagocytic process were demonstrated (FIG. 1G). CAR macrophages were capable of polyphagocytosis, defined as the ability to engulf two or more target cells at once (representative images, FIG. 1H). The ability to redirect macrophage phagocytosis against two additional CAR targets—mesothelin and HER2—were demonstrated with CARs based on scFv from clones SS1 or 4D5, respectively (FIG. 1I). Together these data demonstrated that CD3ζ based CARs can direct the phagocytic activity of macrophages and provided support for subsequent efforts to translate this platform to primary human macrophages.

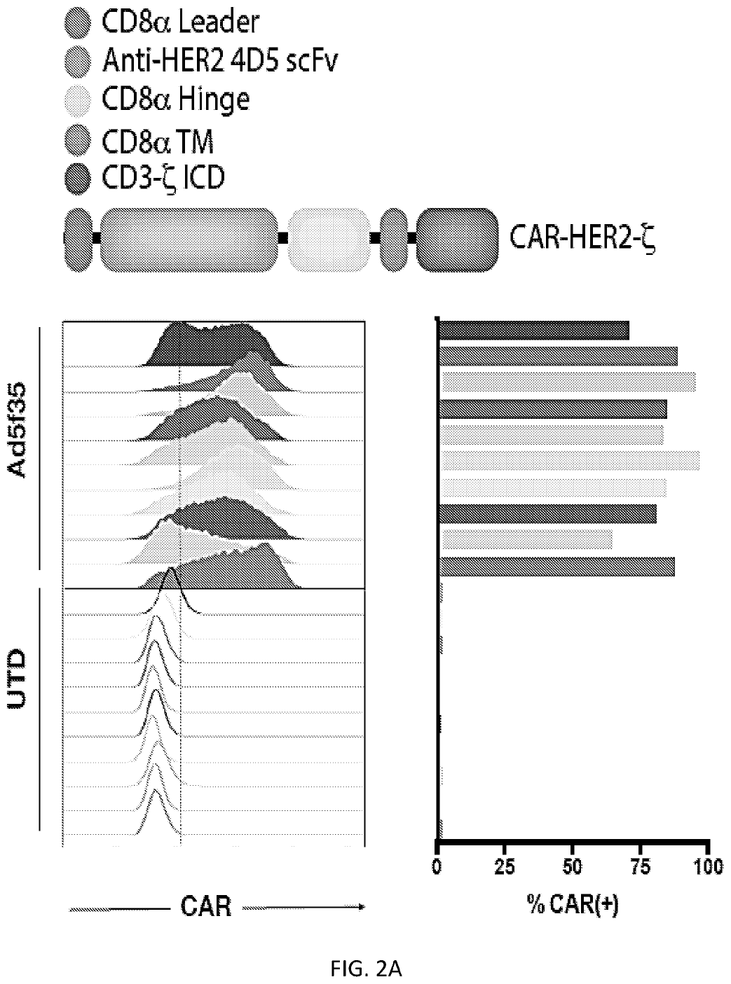

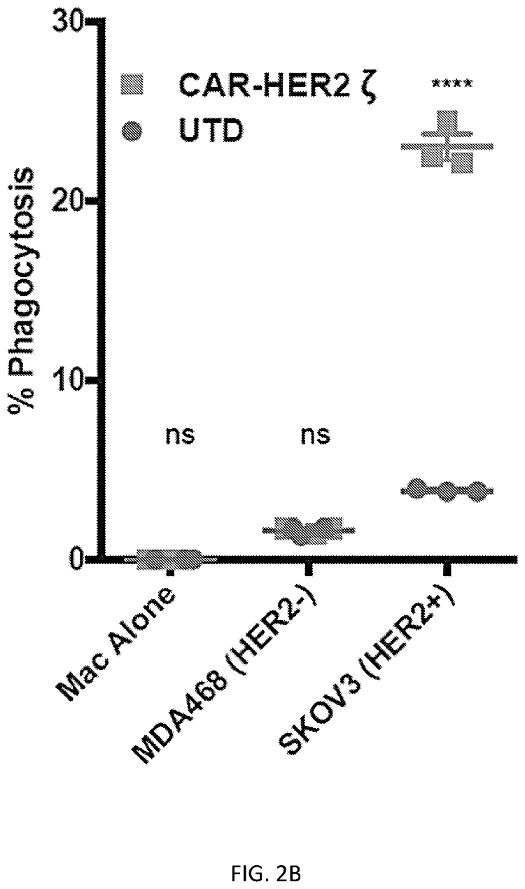

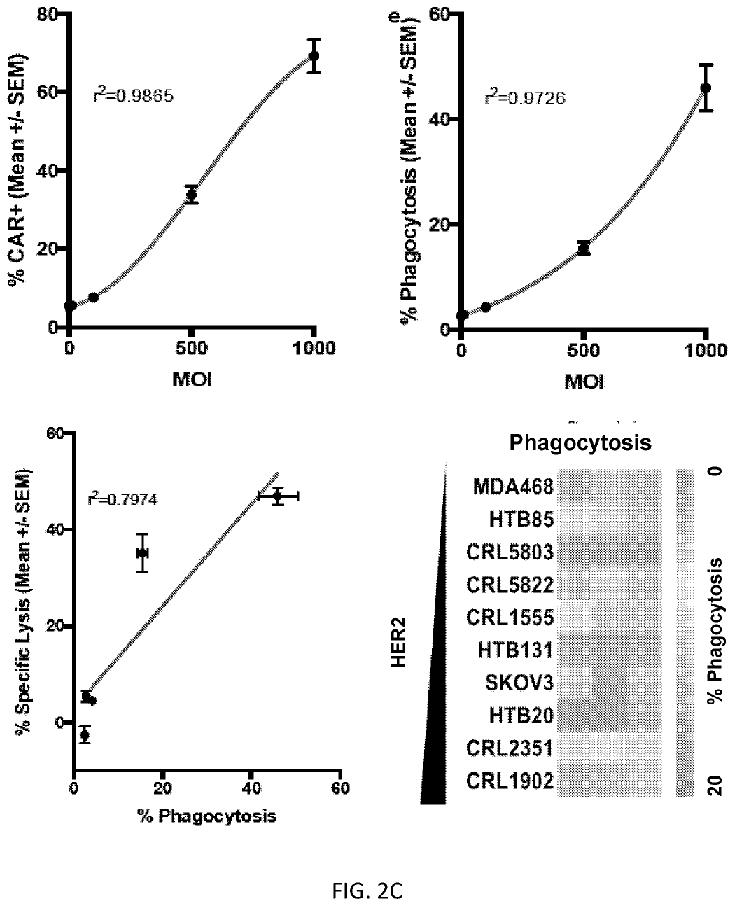

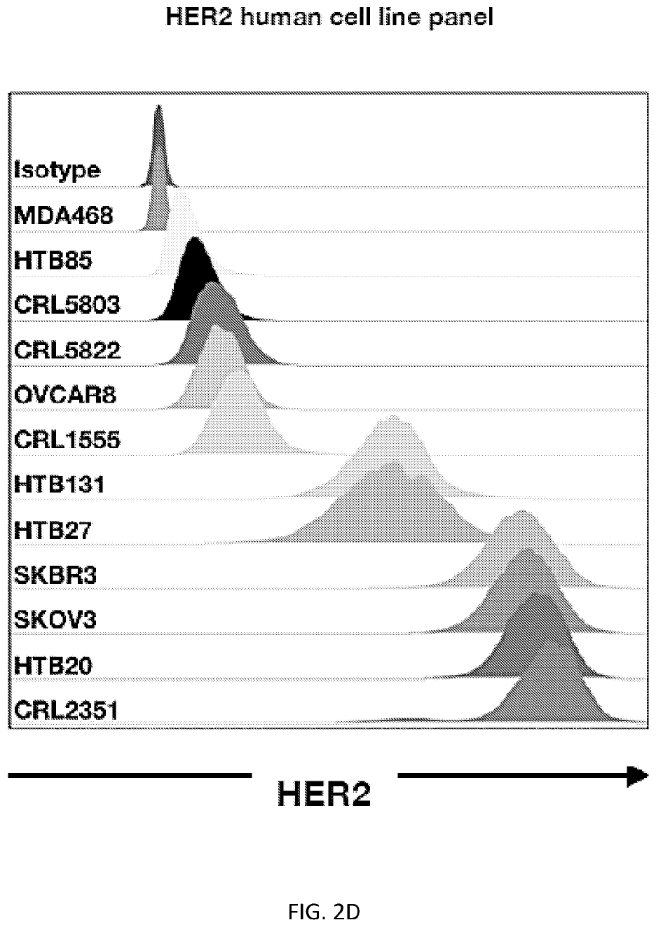

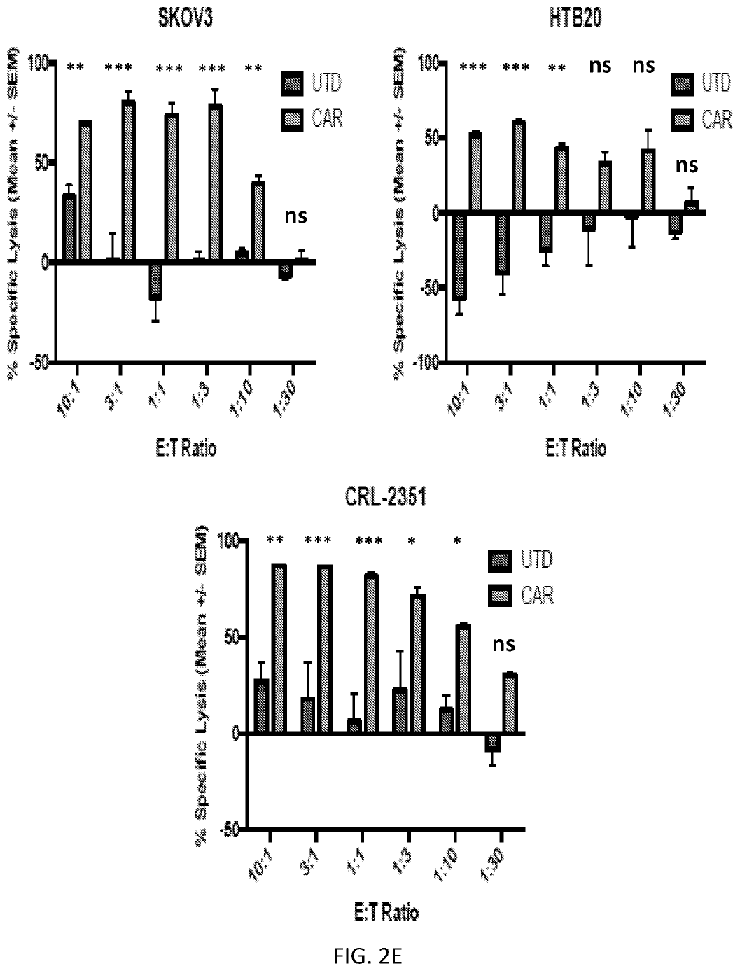

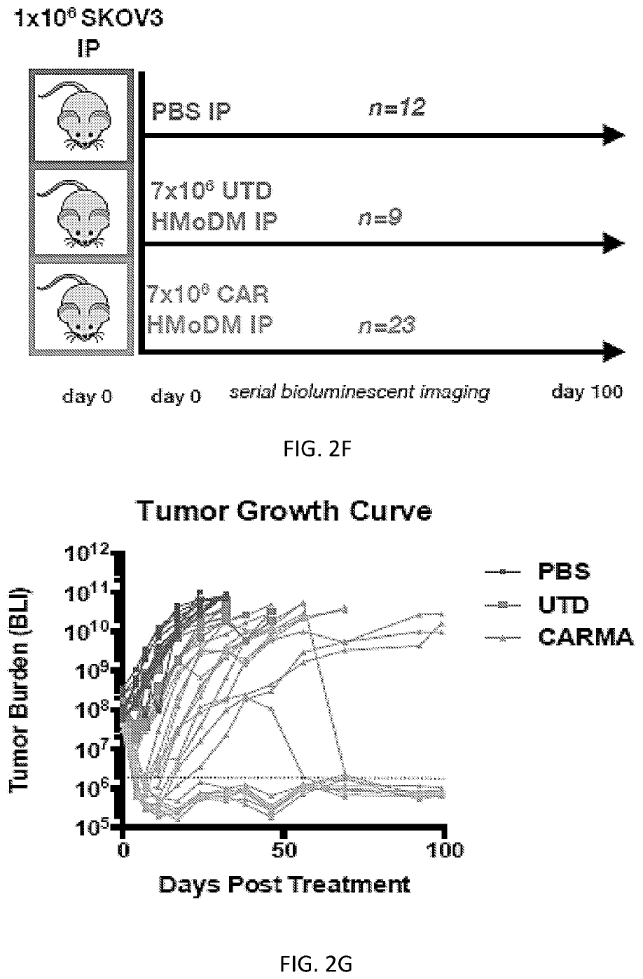

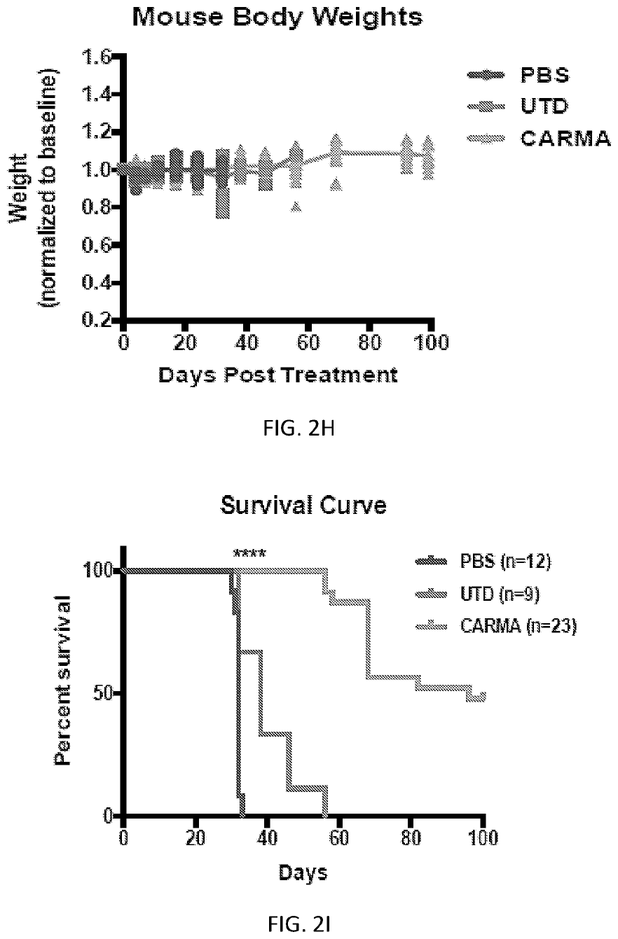

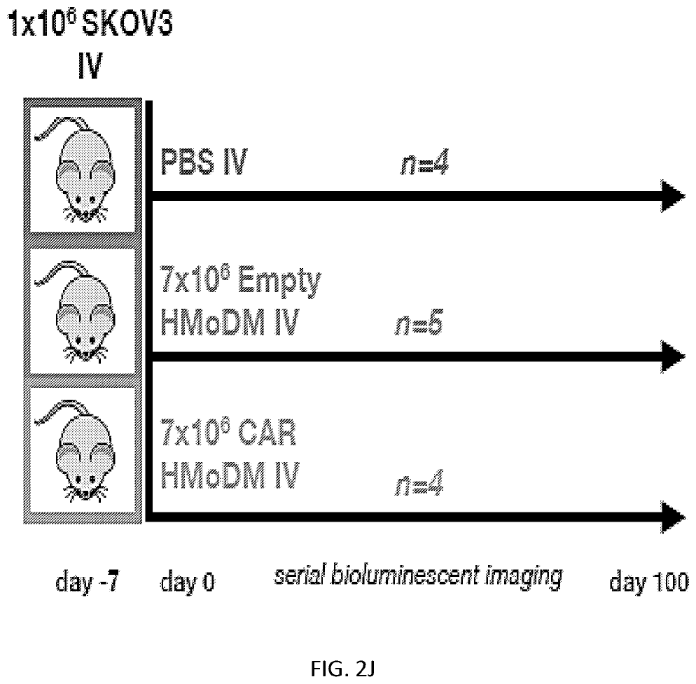

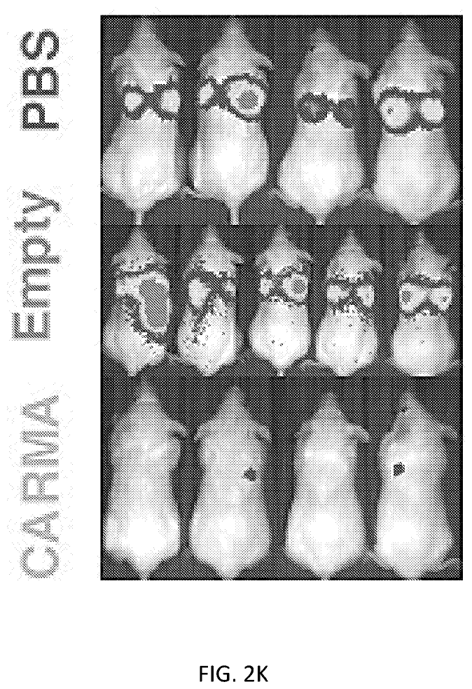

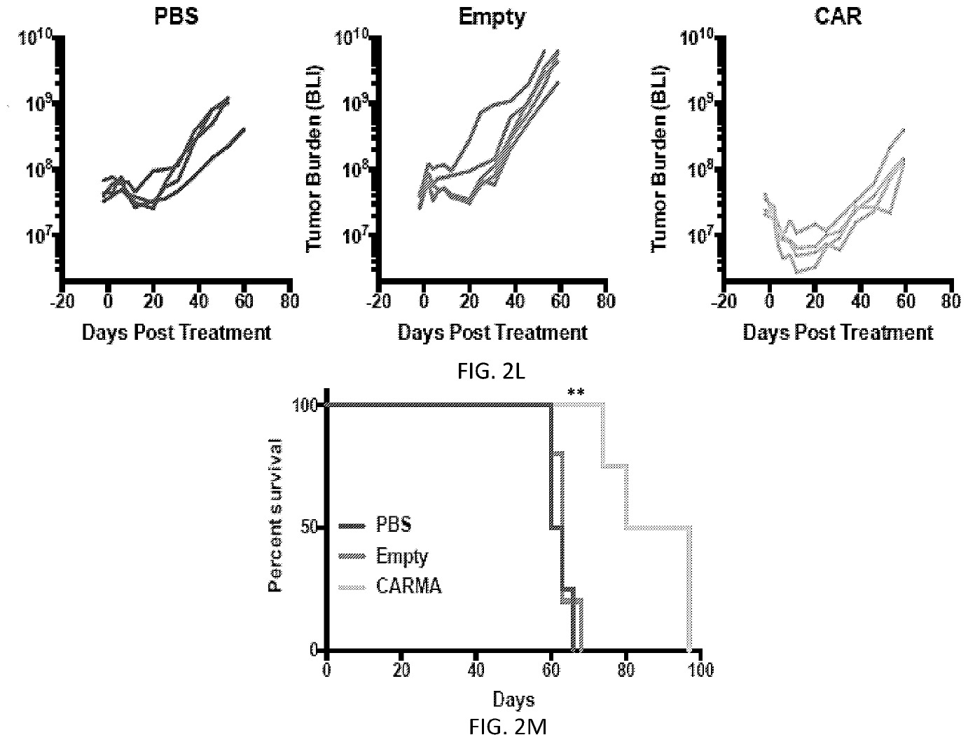

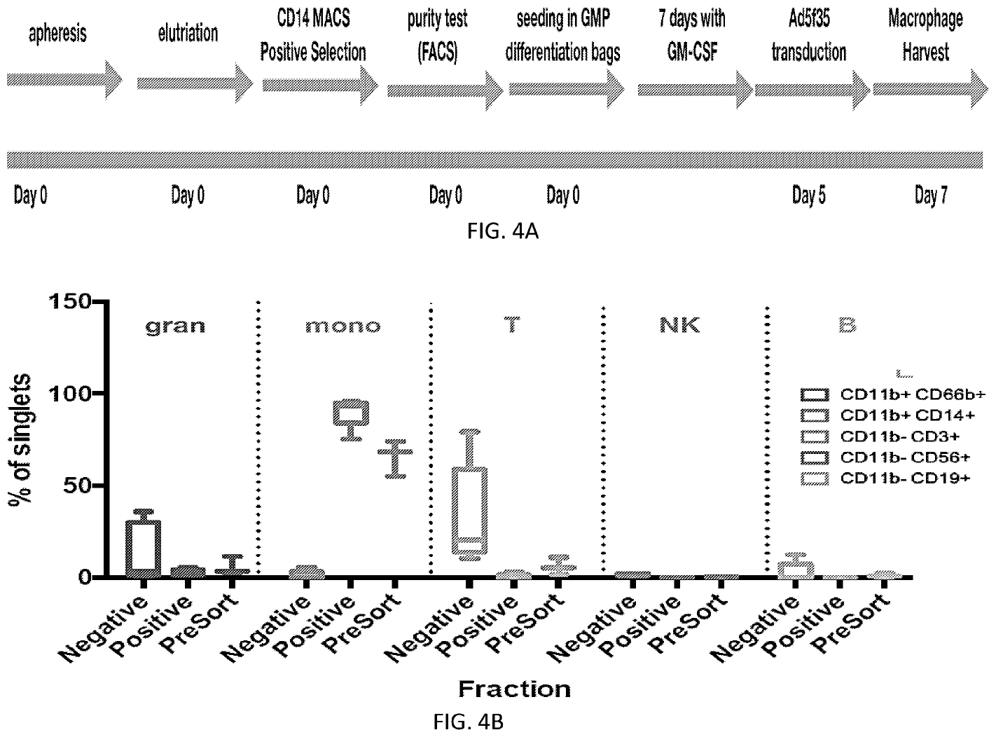

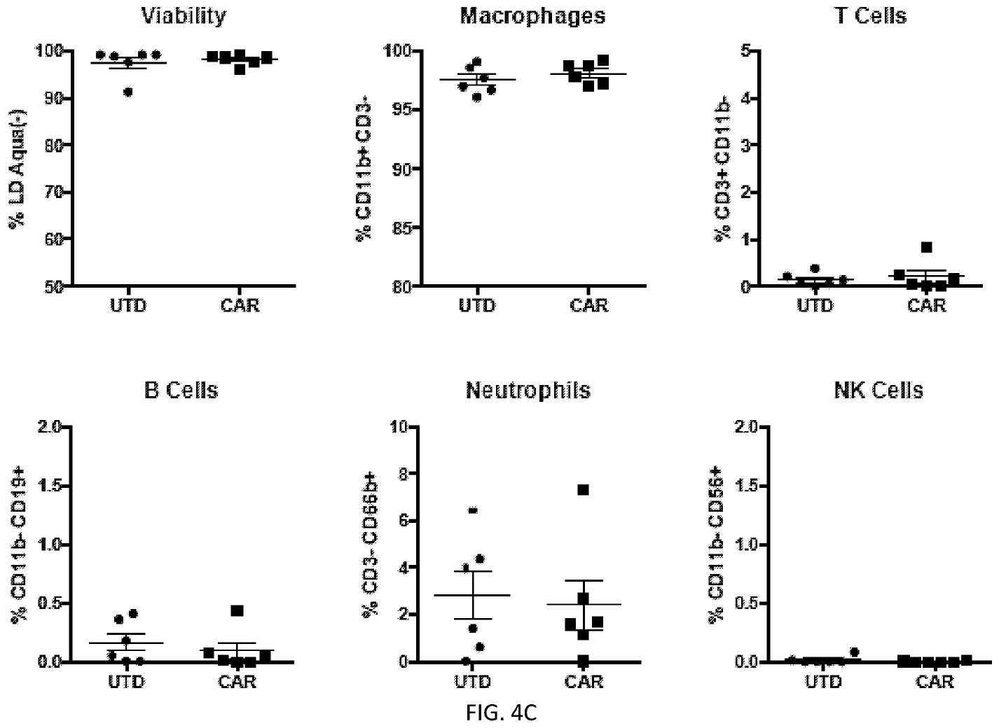

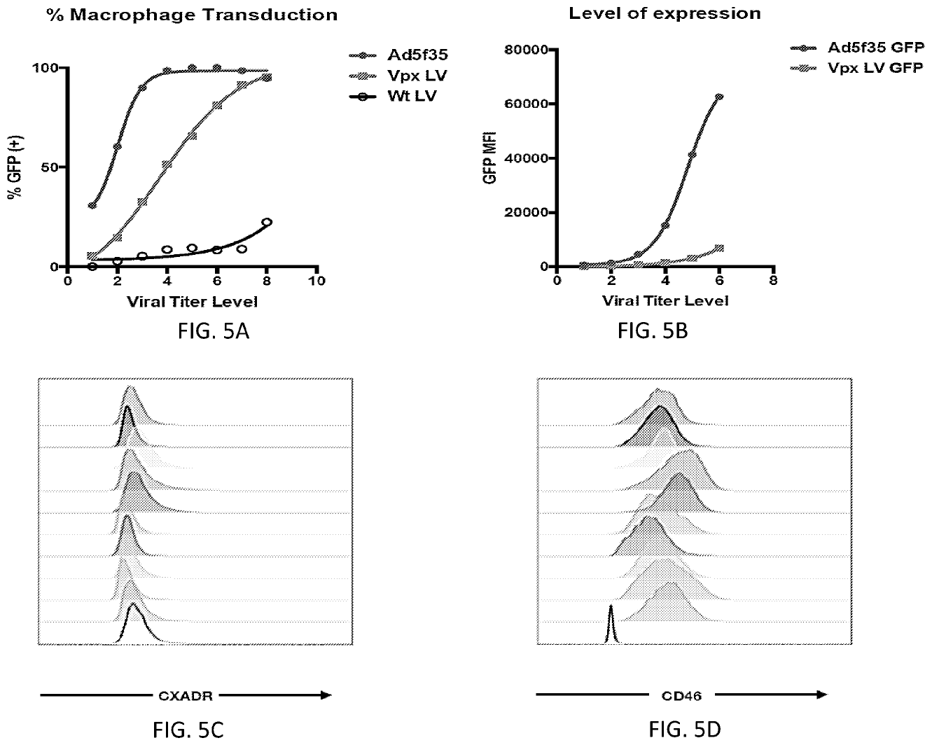

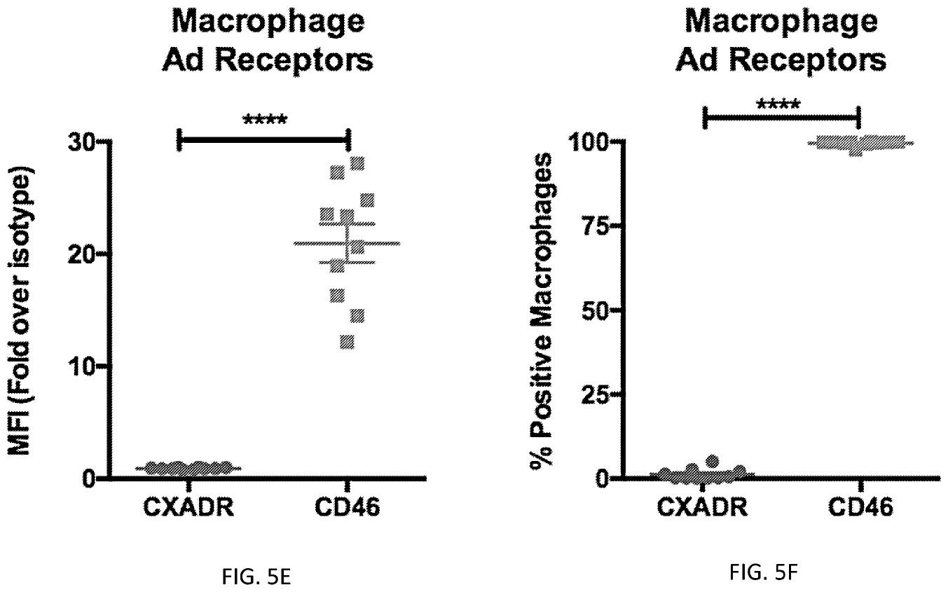

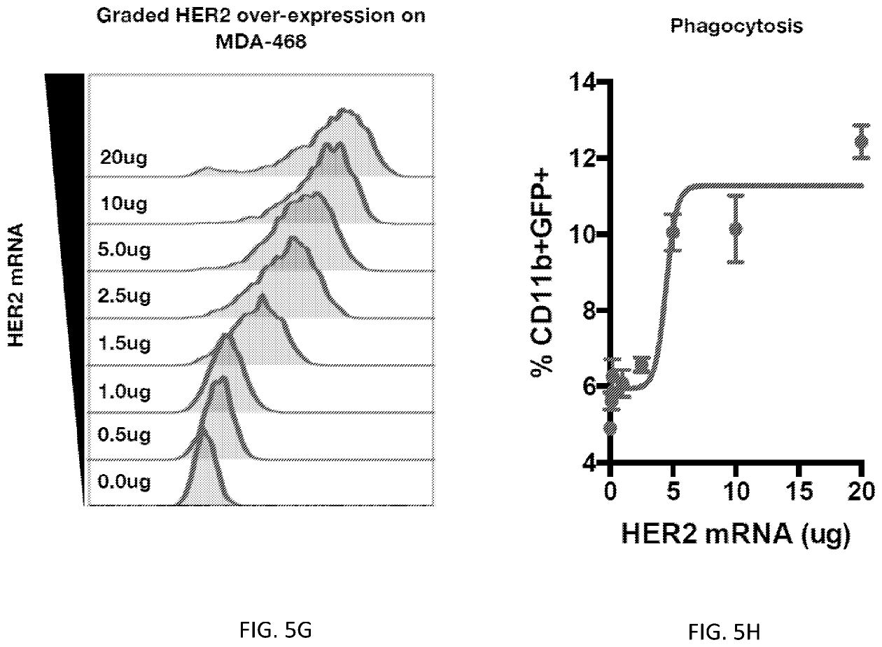

Primary human macrophages were generated by differentiating peripheral blood human CD14+ monocytes with recombinant human GM-CSF for 7 days (FIG. 4A-4C). Since transduction of primary human monocytes and macrophages is challenging, a broad array of integrating and non-integrating viral vectors were tested including lentivirus, Vpx modified lentivirus (Bobadilla, S., et al. (2013) Gene Ther. 20, 514-520), a panel of AAV serotypes, and Ad5f35 (FIG. 5A-5B). Given the low transduction efficiency of standard third generation lentiviral and AAV vectors, and the high MOIs and viral volumes required for Vpx-lentivirus, Ad5f35, a modified chimeric fiber adenoviral vector, was chosen for further study. This vector was selected because of the differential expression on human macrophages of the Ad5 and Ad5f35 docking receptors, CXADR and CD46, respectively (FIG. 5C-5F). Given the high transduction efficiency of Ad5f35-GFP, a CD3ζ-based anti-HER2 CAR was engineered into an Ad5f35 backbone and production of CAR-encoding vector was demonstrated. The vector was capable of transducing human macrophages at a high rate of efficiency and reproducibility across ten normal donors (FIG. 2A). The resultant primary human anti-HER2 CAR macrophages demonstrated antigen-specific phagocytosis (FIG. 2B). The level of tumor phagocytosis and killing correlated with the level of CAR expression, and phagocytosis as measured by a FACS based assay correlated with luciferase-based cytotoxicity (FIG. 2C). Given potential concerns around the low level HER2 expression on normal tissues, a dose response association between antigen density and phagocytic activity was demonstrated by electroporating a HER2-negative cell line with increasing amounts of in vitro transcribed HER2 mRNA and measuring phagocytic activity (FIG. 5G-5H). This was confirmed using a panel of human cancer cell lines with graded expression of HER2 and a clear correlation between antigen density and phagocytic activity was demonstrated (FIG. 2D). Anti-HER2 CAR macrophages mediated dose dependent killing of several HER2 high cancer cell lines in vitro (FIG. 2E).

The in vivo anti-tumor activity of CAR macrophages was tested using two distinct models and routes of administration. The immunodeficient triple transgenic mouse strain NOD scid_{yc}^{-/-} hIL3-hGMCSF-hSF (NSGS) were used for all in vivo xenograft experiments Wunderlich, M. et al. (2010) Leukemia 24, 1785-1788). In the first model, NSGS mice were injected intraperitoneally (IP) with luciferase expressing SKOV3 and treated 2-4 hours later with a single IP injection of phosphate buffered saline (PBS), untransduced (UTD), or anti-HER2 CAR macrophages (CAR) (FIG. 2F). CAR, but not control UTD macrophages, led to significant tumor rejection in the majority of treated mice as demonstrated by serial bioluminescent imaging over 100 days (FIG. 2G). The treatment was not associated with significant toxicity as demonstrated by body weights (FIG. 2H) and led to significantly improved overall survival in the CAR treatment group (median survival 96 (CAR) vs 38 days (UTD), p<0.0001) (FIG. 2I). In the second approach, metastatic disease was modeled by injecting SKOV3 intravenously (IV) and allowing 7 days for engraftment. Mice then received a single IV injection of PBS, macrophages transduced with empty Ad5f35 vector (Empty), or anti-HER2 CAR macrophages (FIG. 2J). CAR treated mice demonstrated a significant reduction in tumor burden (FIGS. 2K-2L). Though transient, a single infusion of CAR macrophages led to a significant improvement in overall survival (median survival 88.5 (CAR) vs 63 days (Empty), p=0.0047) (FIG. 2M). Collectively, these results demonstrated that CAR macrophages can be efficiently generated from human peripheral blood to exhibit targeted anti-tumor activity in vitro and in murine xenograft models.

### Example 2—Exposure to Ad5f35 Induces a Pro-Inflammatory Phenotype

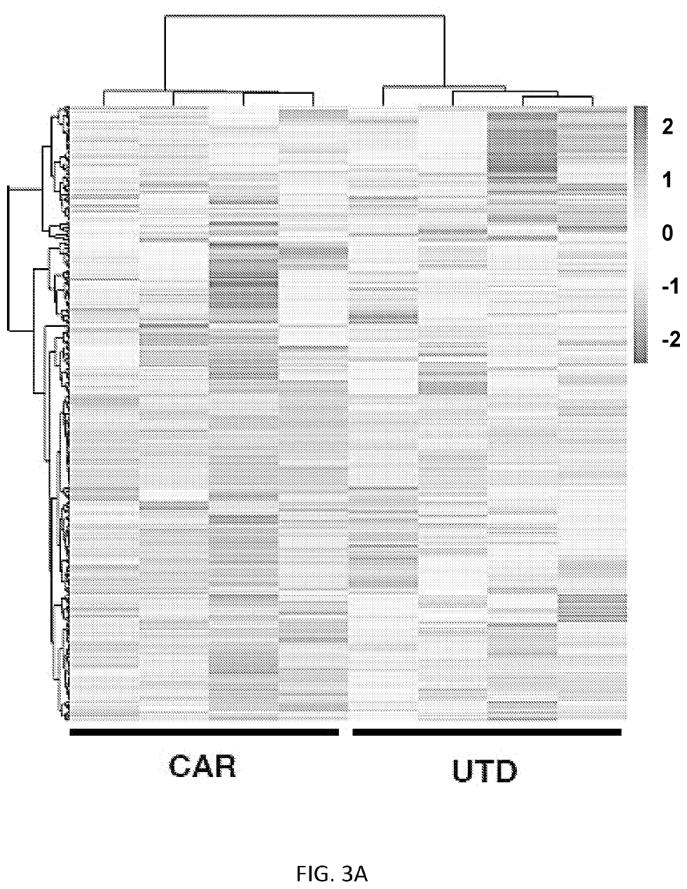

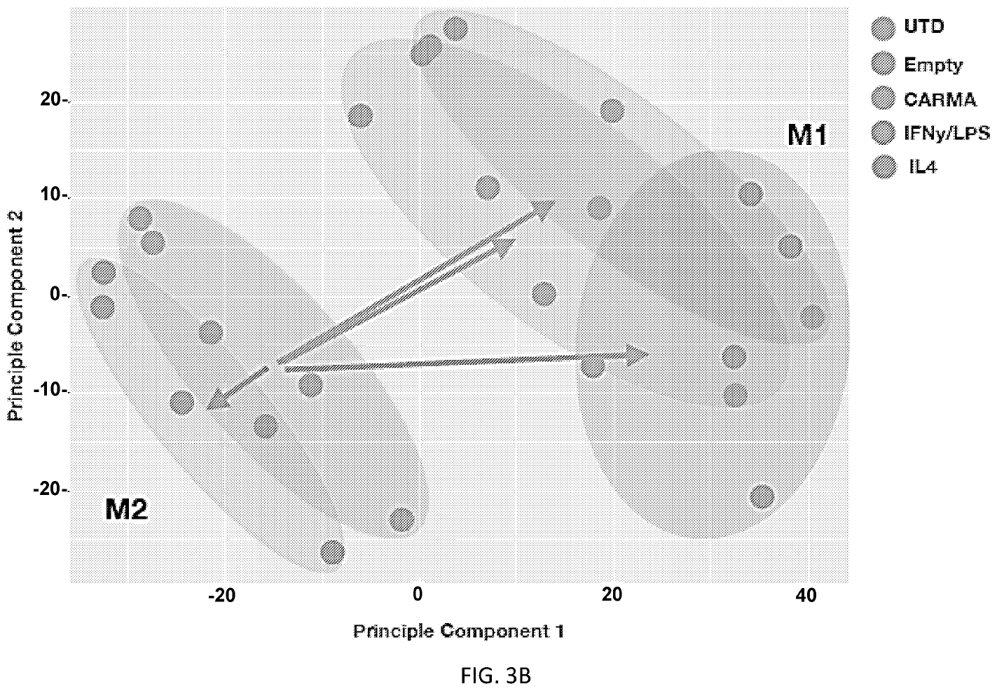

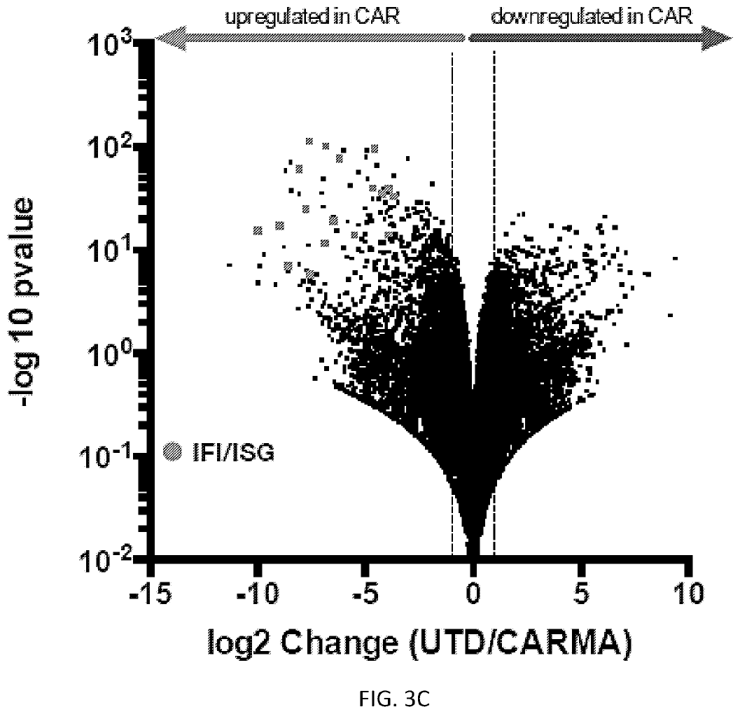

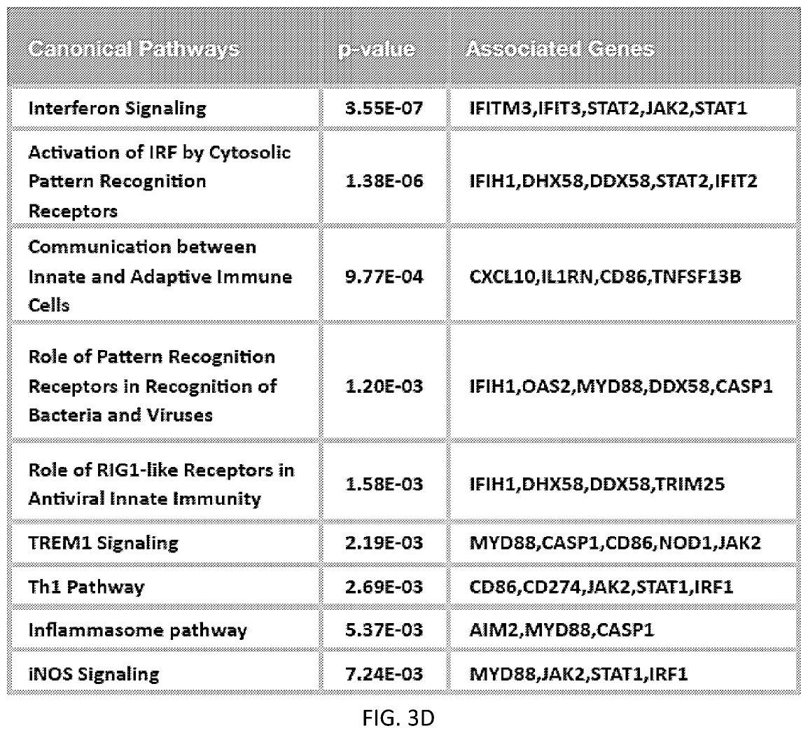

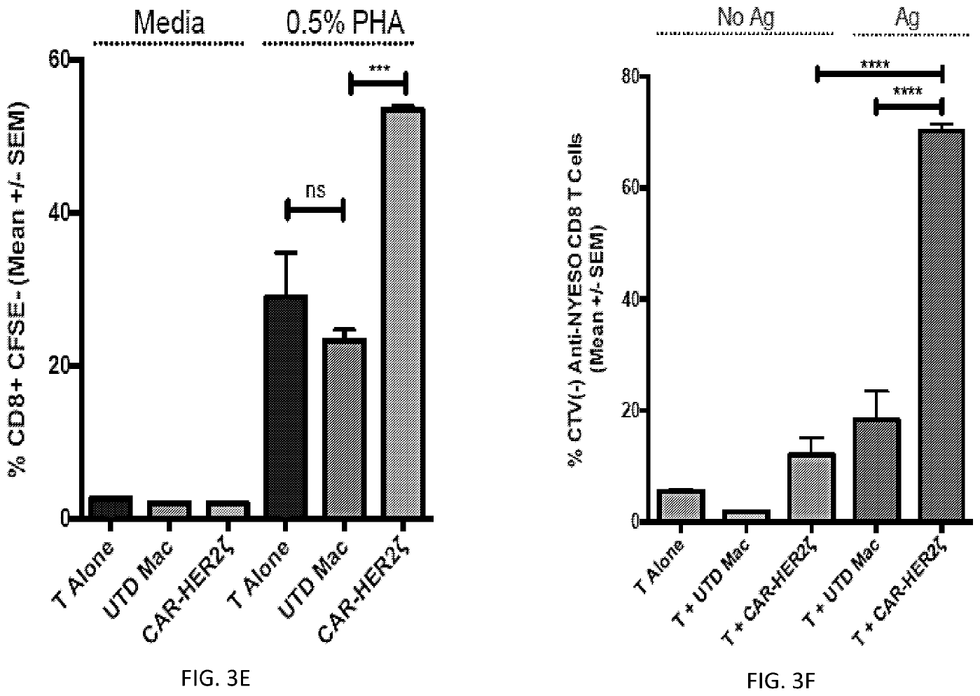

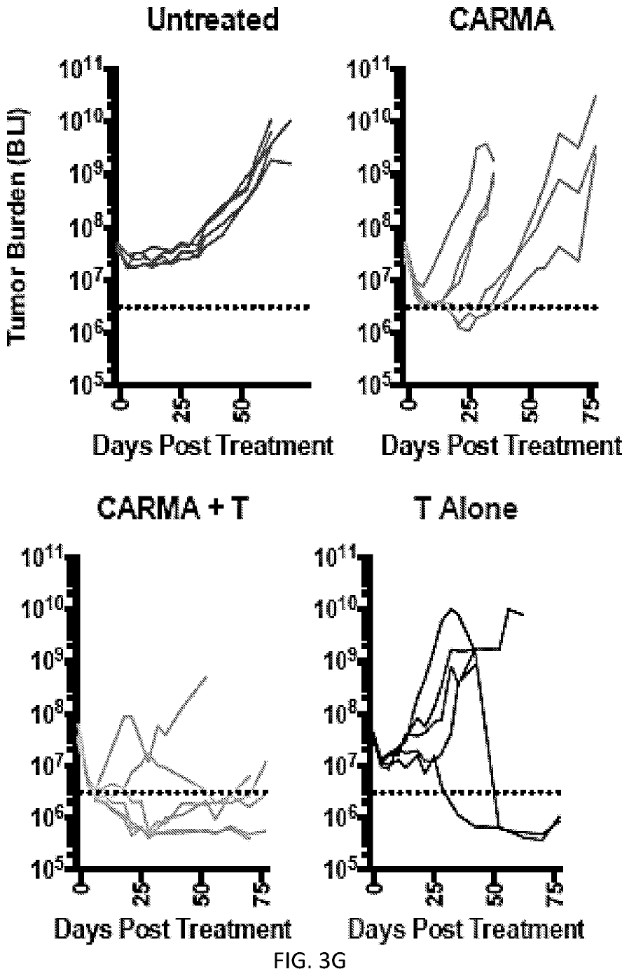

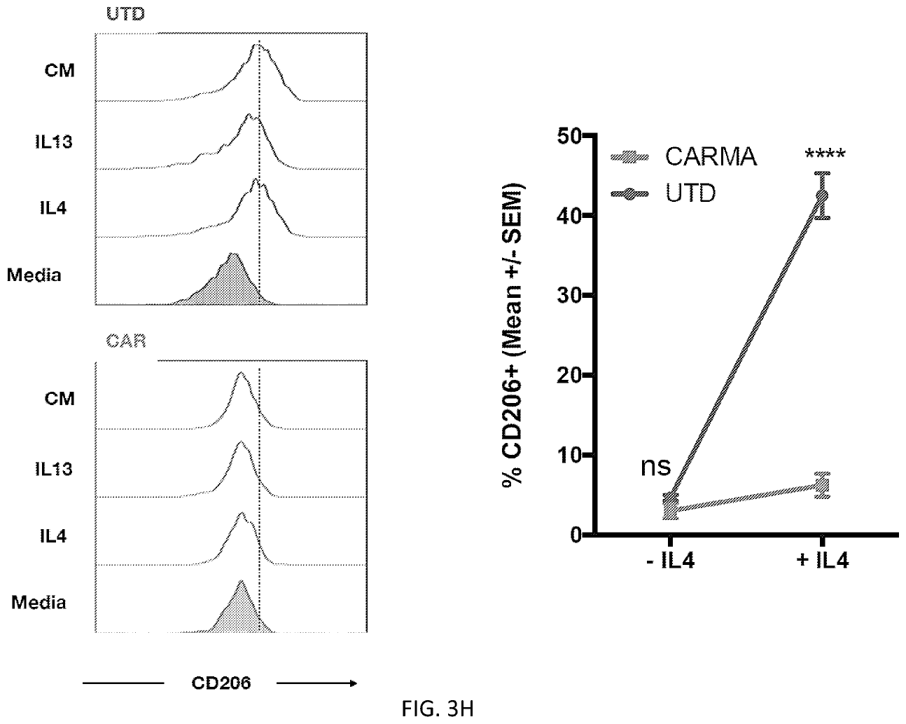

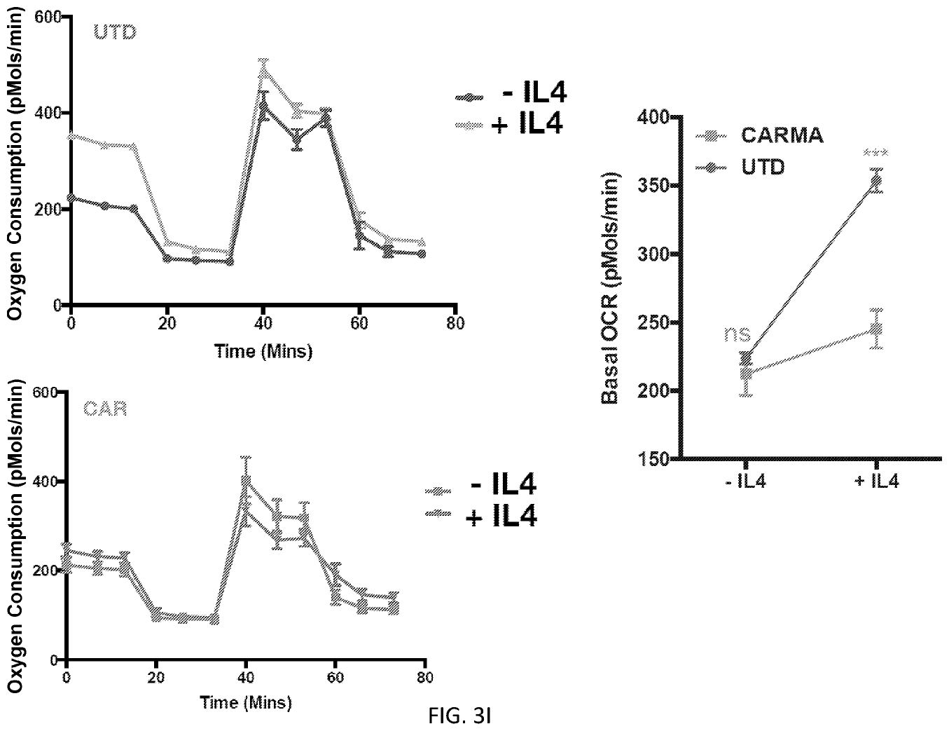

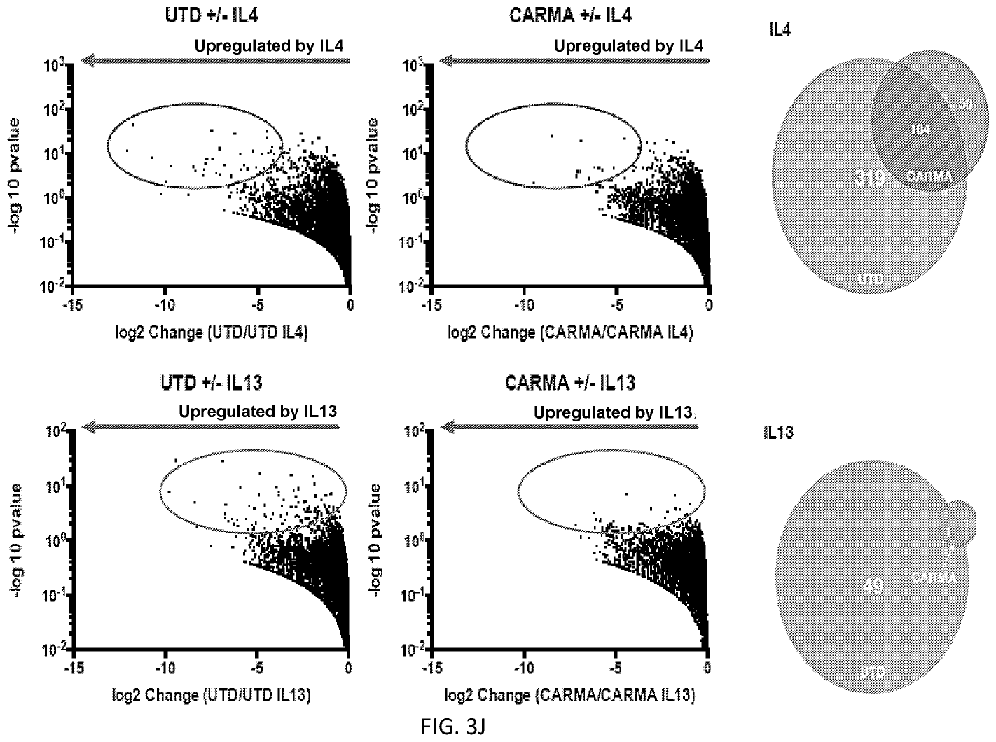

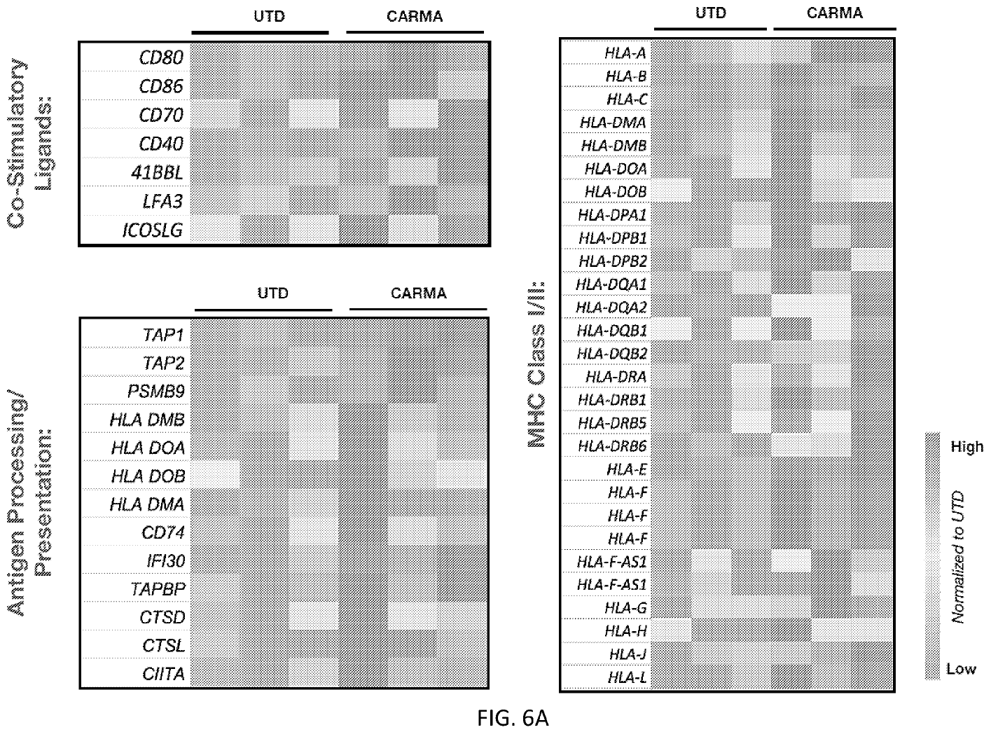

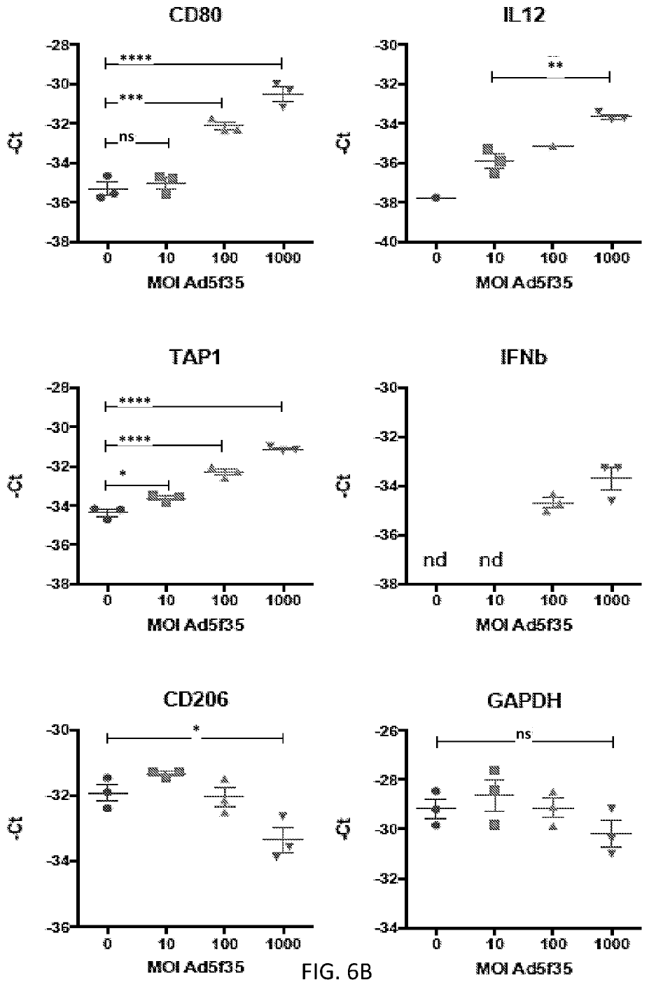

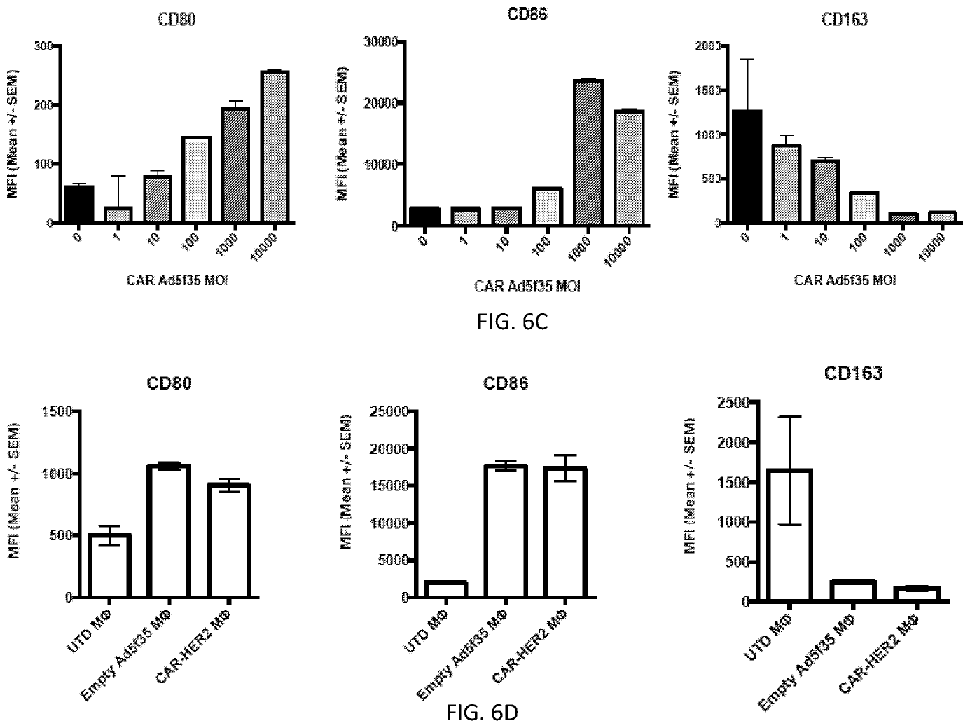

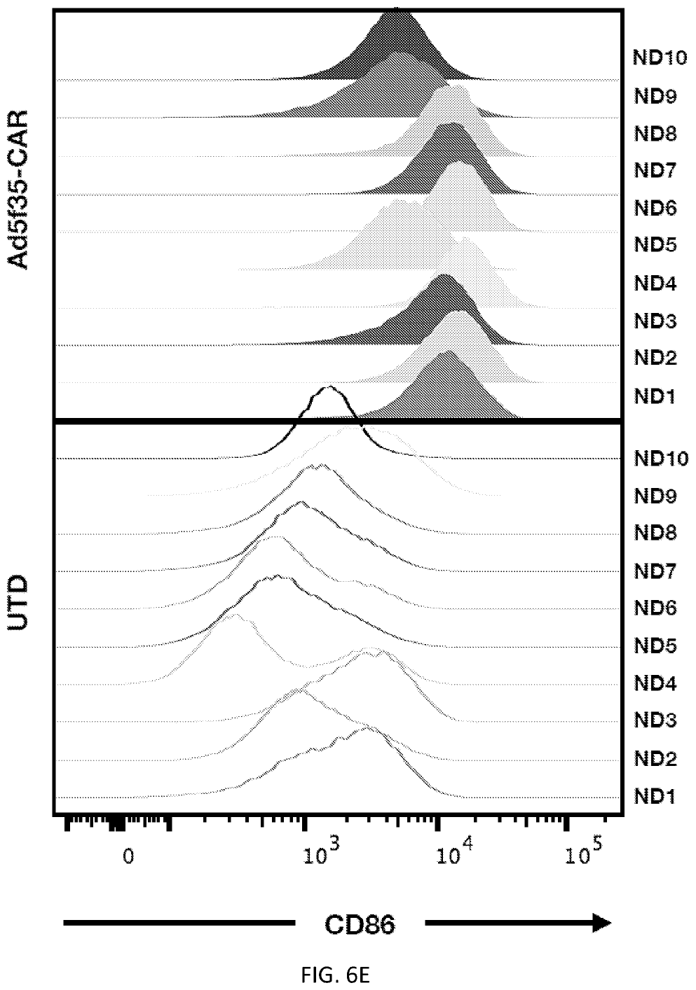

Macrophage phenotype is plastic and can change in response to cytokines, pathogen associated molecular patterns, metabolic cues, cell-cell interactions, and tissue-specific signals. It was hypothesized that exposure to Ad5f35, a double stranded DNA virus, may induce a pro-inflammatory (M1-like) phenotype. Using non-biased hierarchical clustering of macrophage transcriptomes from four human donors, transduced macrophages clustered distinctly from control untransduced macrophages, demonstrating a phenotypic shift (FIG. 3A). Furthermore, when untransduced (UTD), Ad5f35-CAR transduced, empty-vector Ad5f35 transduced (Empty), IFNy/LPS (classically activated, M1) stimulated, or IL4 (alternatively-activated, M2) stimulated macrophage transcriptomes from five human donors were subject to non-biased principal component analysis, adenovirally transduced macrophages clustered toward the classically-activated and away from the alternatively-activated macrophages, regardless of CAR expression (FIG. 3B). Transduction led to the induction of many interferon associated genes, consistent with a classically-activated M1 phenotype (FIG. 3C; IFI, interferon induced; ISG, interferon stimulated gene). Furthermore, a myriad of co-stimulatory ligand, antigen processing/presentation, and MHC-Class I/II genes were induced upon transduction (FIG. 6A). Unbiased Ingenuity Pathway Analysis demonstrated the induction of M1 associated pathways, such as interferon, pattern recognition receptor, Th1, RLR, JAK1/JAK2, and iNOS signaling (FIG. 3D). The induction of a pro-inflammatory M1 phenotype was validated by RT-qPCR and flow cytometry, demonstrating an MOI dependent response (FIGS. 6B-6C). The induction and repression of these markers was equivalent between CAR and empty vector Ad5f35, validating that the phenotype was induced by the vector and not related to expression of CAR in macrophages (FIG. 6D). Ad5f35 induced M1-induction was validated on macrophages from 10 human donors (FIG. 6E).

### Example 3—CAR Macrophages Exhibit Ability to Co-Stimulate and Present Antigens to T Cells

Given the upregulation of co-stimulatory ligand and antigen processing/presentation genes, and the fact that macrophages are professional antigen presenting cells (APCs), the capacity for CAR macrophages to co-stimulate and present antigens to T cells was tested. CD8+ T cells stimulated with phytohemagglutinin (PHA) in vitro, a non-specific source of signal 1, proliferated significantly more in the presence of transduced than untransduced macrophages (FIG. 3E). To test the capacity for Ad5f35 transduced macrophages to process and present antigen, macrophages were transduced with the tumor-associated antigen NY-ESO1 and the HLA-A2*01 molecule. Macrophages were then transduced with Ad5f35, or not (UTD), and co-cultured with transgenic anti-NY-ESO-1 (1G4) TCR+ autologous T cells. Ad5f35 transduced NY-ESO1-expressing macrophages induced significantly more proliferation of 1G4+ CD8+ T cells than NY_ESO1-expressing control macrophages or Ad5f35 transduced macrophages that lacked NY-ESO1 (FIG. 3F). In order to test the potential of CAR macrophages to stimulate T cells in vivo, NSGS mice were engrafted with a disseminated SKOV3 model and treated with CAR macrophages, CAR macrophages plus autologous polyclonal T cells (CAR+T), T cells alone, or left untreated. Mice treated with CAR macrophages plus autologous T cells had deeper anti-tumor responses (FIG. 3G) and generated more xenogeneic graft-versus host disease than the control conditions, suggesting that Ad5f35 transduced macrophages stimulated autologous T cells in vivo.

### Example 4—Macrophages Transduced with Ad5f35 are Less Responsive to M2-Inducing Cytokines

Previous studies have shown that IFN-γ induced M1 macrophages repressed M2 genes via epigenetic reprogramming. In the present study, phenotype plasticity was tested by challenging control or transduced human macrophages with two canonical M2 inducing cytokines—IL-4 or IL-13. Upon stimulation with IL-4, IL-13, or SKOV3 conditioned media, UTD, but not transduced macrophages, upregulated the M2 marker CD206 (FIG. 3H). Furthermore, upon stimulation with IL-4, UTD, but not transduced macrophages, increased their basal oxygen consumption rate as expected from IL-4 induced M2 macrophages (FIG. 3I). Transcriptome analysis revealed significantly fewer genes induced by IL-4 or IL-13 in transduced as compared to untransduced macrophages (FIGS. 3J-3K). Collectively, these results demonstrated that Ad5f35 induces a potent pro-inflammatory M1 macrophage phenotype during the transduction process, promotes the ability of macrophages to stimulate adaptive immunity, and reduces the responsiveness of macrophages to M2-inducing cytokines.

In conclusion, the findings of Examples 1-4 support the concept that human peripheral blood monocyte derived macrophages can be targeted to exert a potent anti-tumor effector function via the introduction of a CAR. It was demonstrated that human macrophages can be engineered with high efficiency using Ad5f35, and HER2-redirected human CAR macrophages reduced tumor burden and prolonged overall survival in xenograft models. Furthermore, the data show that Ad5f35 transduction polarized macrophages toward a unique pro-inflammatory/anti-tumor M1 phenotype and reduced their susceptibility to immunosuppressive M2-inducing cytokines. Taken together, these results introduce CAR macrophages as a novel cell therapy platform for the potential treatment of human cancer.

### Example 5—CAR-M Push M2 Macrophages Toward M1 Polarization

The data presented in this Example establish that administration of CAR-M to M2 macrophages pushes M2 macrophages toward an M1 phenotype. Primary human monocyte derived macrophages from 3 distinct human donors were polarized toward 4 different classifications of M2—M2a, M2b, M2c, and M2d. These are the four M2 subtypes studied in the literature, and represent the spectrum of M2 macrophage polarization.

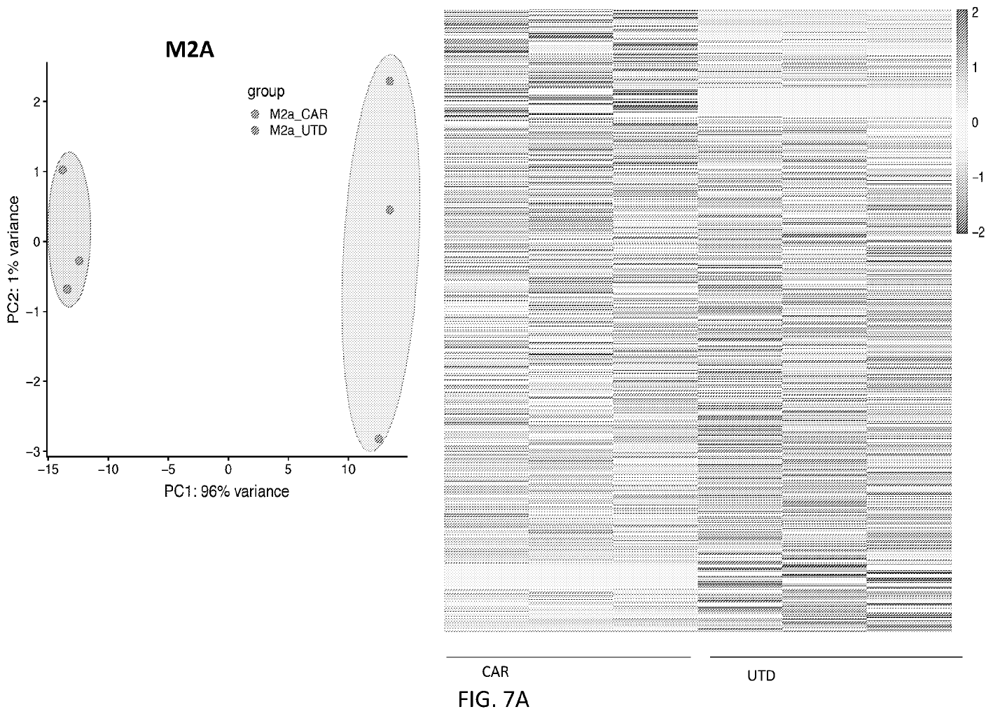

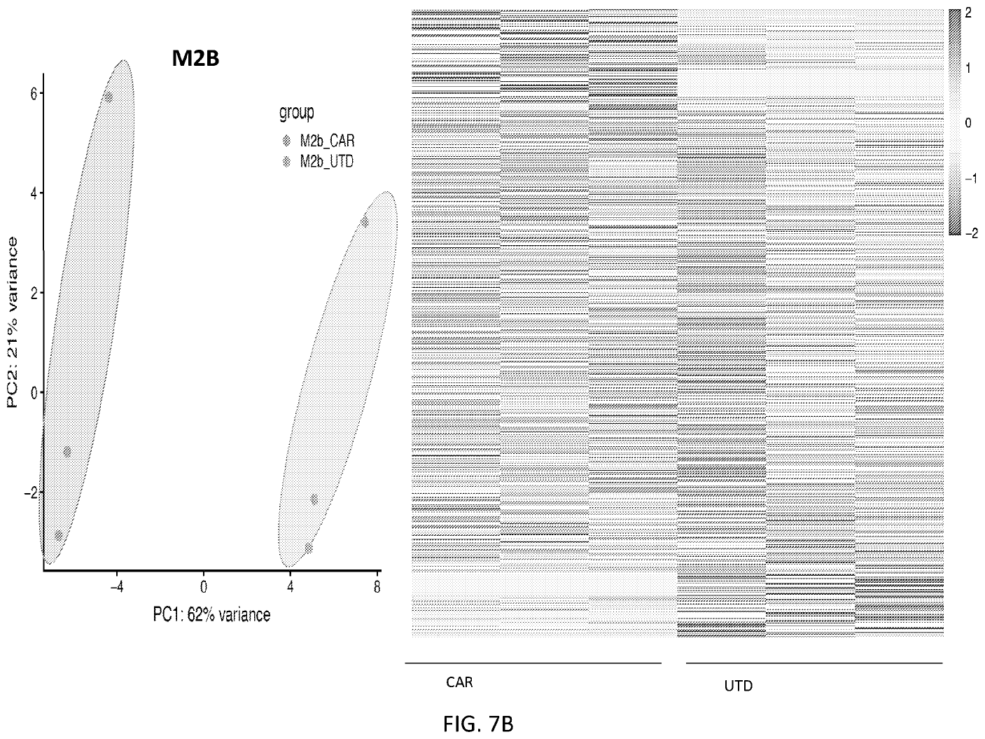

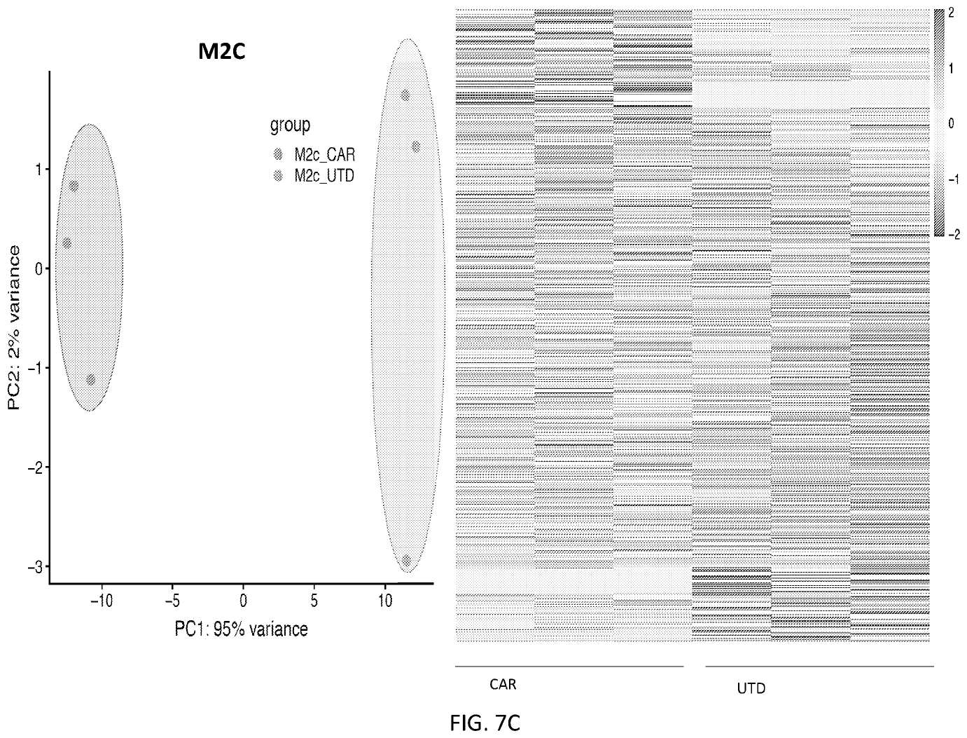

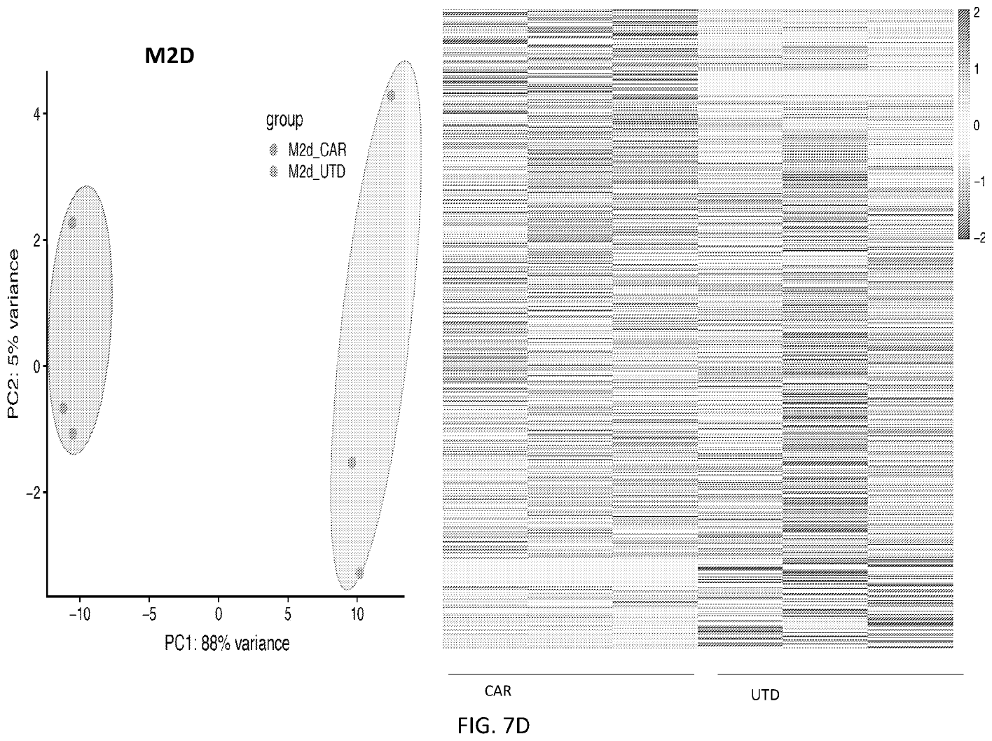

M2 macrophages were challenged with conditioned media generated from control untransduced (UTD) or CAR macrophages (CAR-M). After exposure to control or CAR-M conditioned media, M2 macrophage RNA was collected and subject to RNA sequencing and bio-informatics analysis. As shown in the left-hand graphs of FIGS. 7A-7D, principle component analysis illustrates that CAR-treated M2 macrophages were phenotypically distinct from control-treated M2 macrophages and clustered apart from each other by treatment. As shown in the right-hand parts of FIGS. 7A-7D, unbiased hierarchical clustering illustrates that CAR-treated M2 macrophages were phenotypically distinct from control-treated M2 macrophages and clustered apart from each other by treatment. This shows that factors secreted by CAR-M induced phenotypic changes in M2 macrophages of all subtypes.

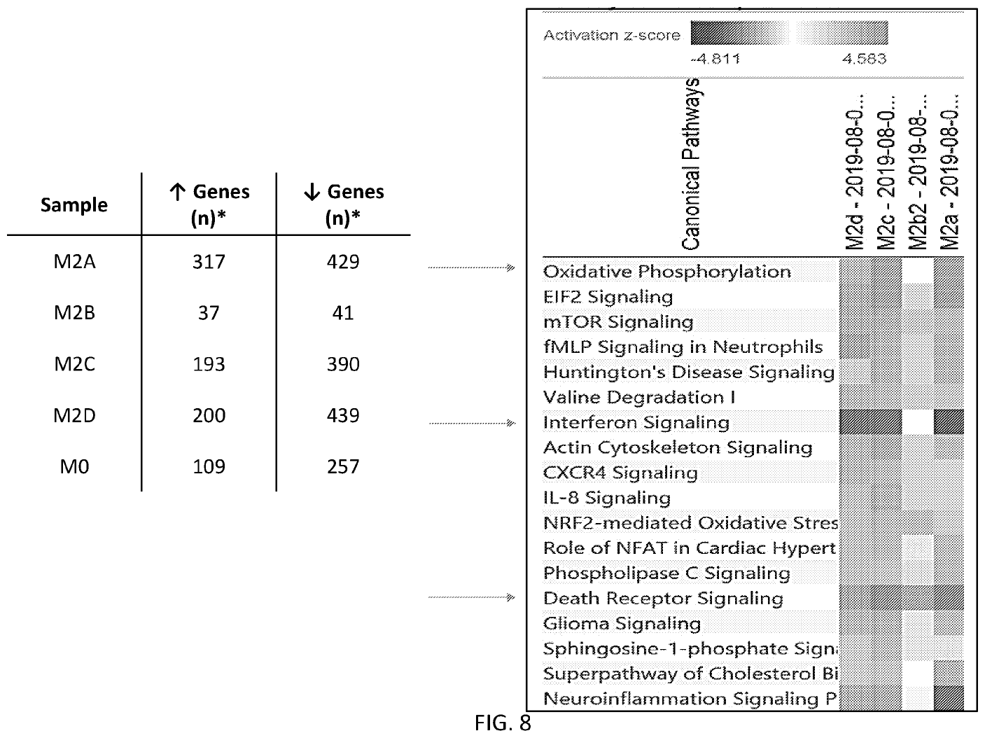

Based on RNA sequencing data, the expression of many genes were upregulated and downregulated in M2 macrophages upon treatment with factors secreted from CAR-treated macrophages (*log FC>1, adj. p-val <0.05) (FIG. 8). The differentially expressed genes (DEG) were analyzed by the Ingenuity Pathway Analysis algorithm. The results demonstrated that CAR-M induced expression of RNAs in M1-associated pathways in M2 macrophages (e.g., interferon signaling), and decreased expression of RNAs in certain M2-associated pathways in M2 macrophages (e.g., oxidative phosphorylation). Importantly, the “Death Receptor Signaling” pathway was upregulated in M2 macrophages treated with CAR-M, suggesting that factors secreted from CAR-M can have anti-M2 macrophage associated properties.

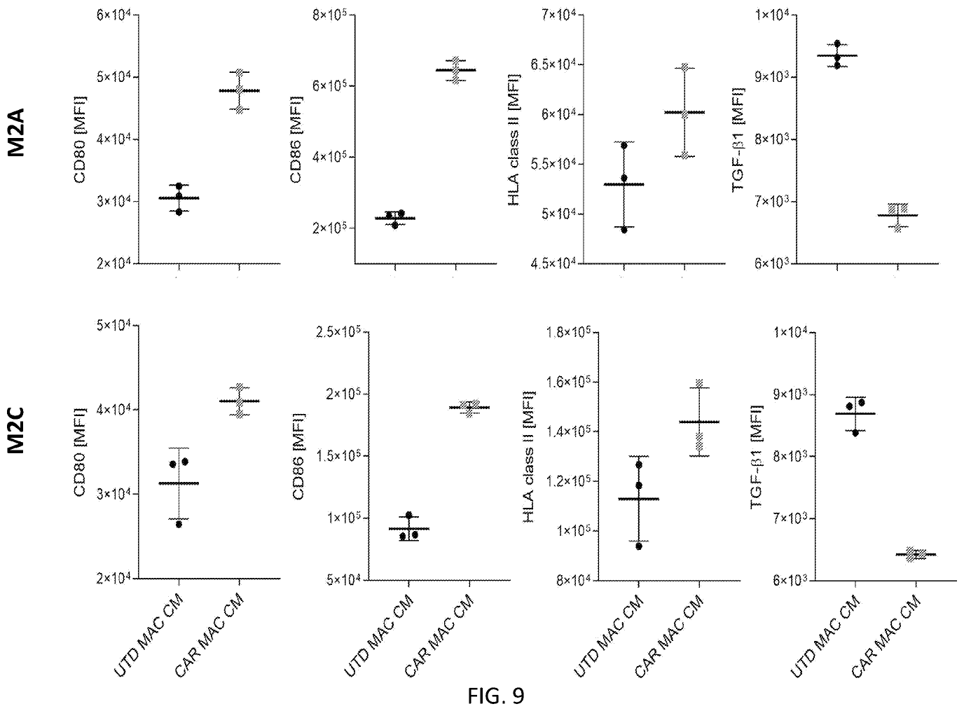

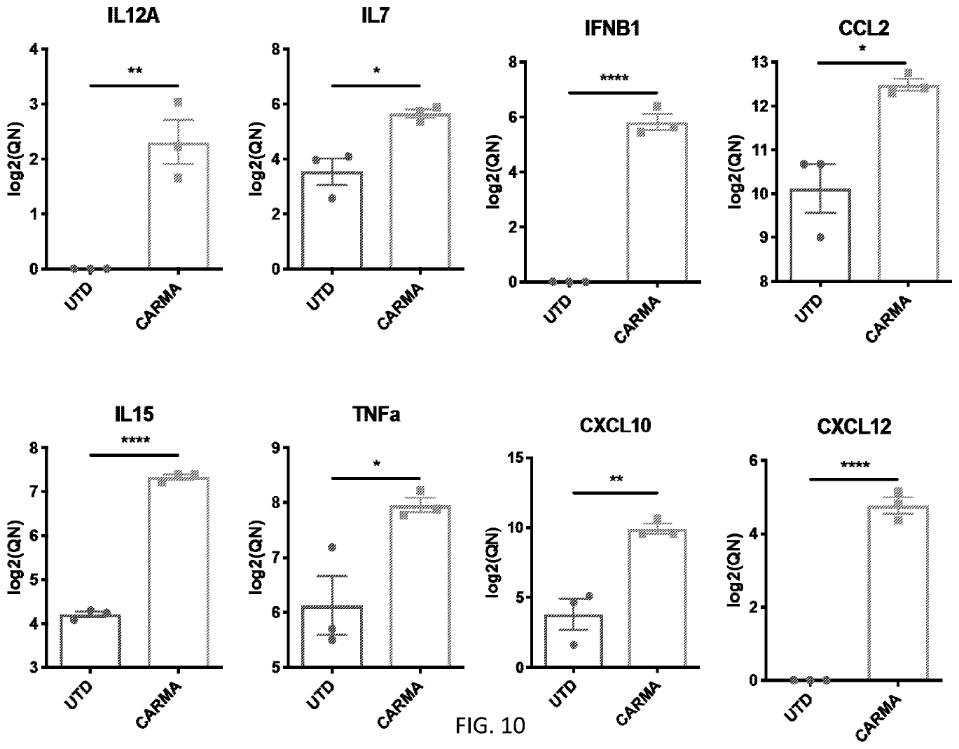

To confirm the results of the RNA-Seq studies, FACS analysis was performed to determine phenotypic changes at the protein level (FIG. 9). FACS results demonstrated the induction of human M1 markers (CD80, CD86, HLA Class II) and downregulation of M2 marker TGF-β1 in M2 macrophages exposed to CAR-M. Taken together with the RNA-Seq results, these data demonstrate that exposure to CAR-M can skew the phenotype of M2 macrophages toward the phenotype of M1 macrophages. Additionally, evaluation of an exemplary gene expression profile of CAR-M demonstrates the induction of a myriad of secreted pro-inflammatory factors that have the potential to activate or skew M2 macrophages toward an M1 phenotype (FIG. 10).

### Example 6—CAR-M Maintain Ability to Kill in Presence of M2 Macrophages

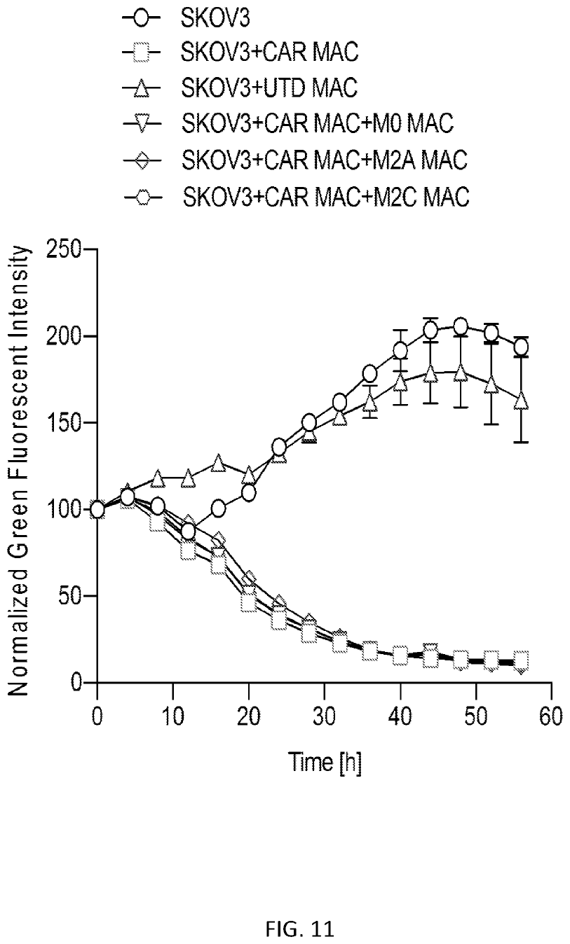

The ability of CAR-M to kill tumor cells in the presence of M2 macrophages of different subtypes was evaluated (FIG. 11). These results showed that SKOV3 cells were killed by CAR-M cells, independent of the presence of M0, M2a, or M2c macrophages.

### Example 7—CAR-M Maintain Ability to Kill Tumor Cells in the Presence of a Human Tumor Microenvironment

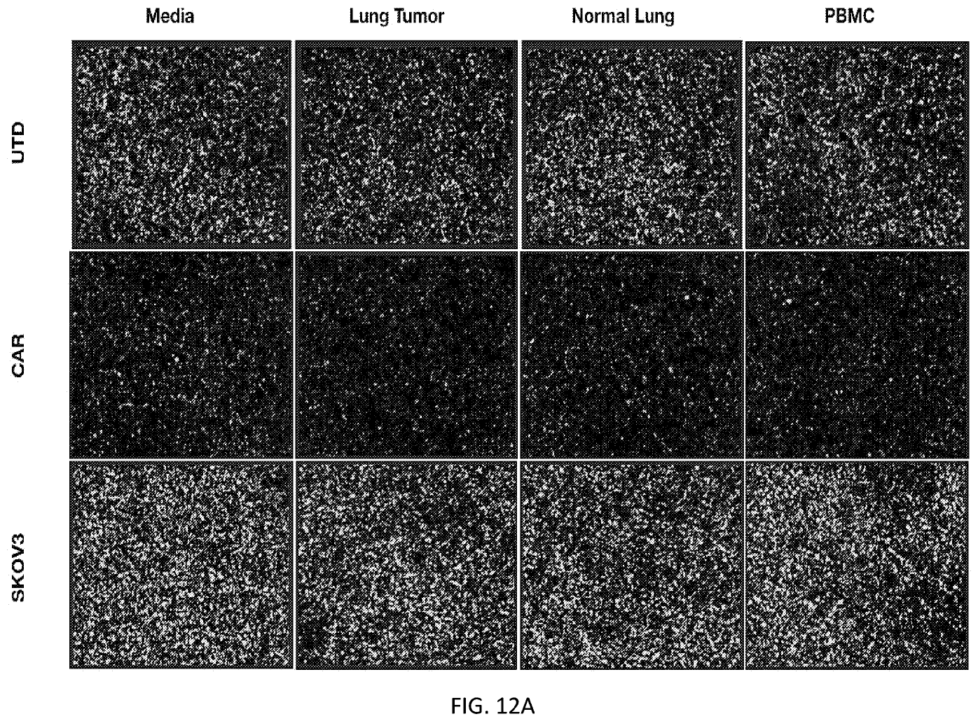

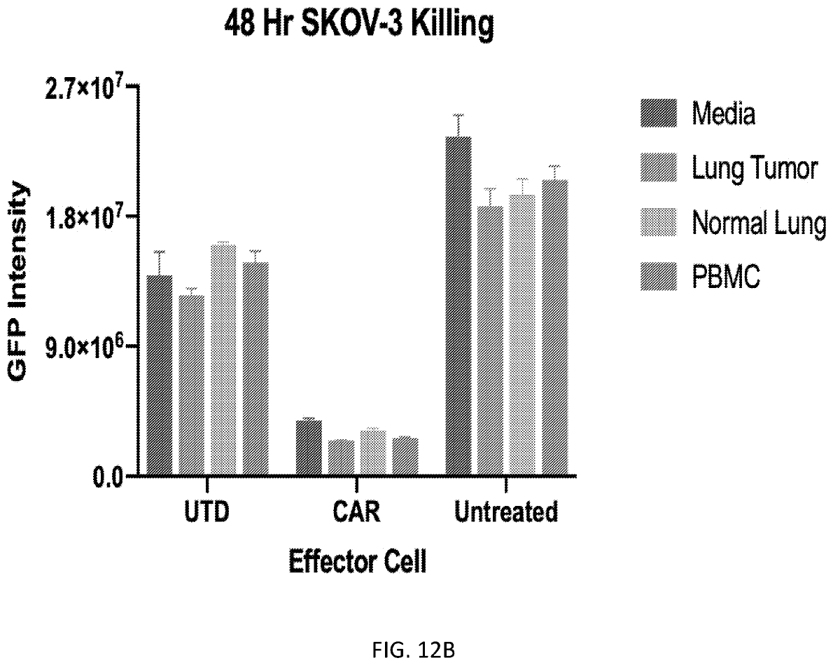

Given that in vitro generated M2 macrophages only model tumor-associated macrophages, the ability of CAR-M to kill tumor cells in the presence of a primary human tumor milieu was examined. In the presence of a single cell human lung tumor microenvironemt suspension, CAR-M maintained their ability to kill tumor cells. Normal lung tissue and PBMCs were used as controls (FIGS. 12A-12B).

### Example 8—CAR-M Maintain an M1 Phenotype in Model Tumor Microenvironment (TME)

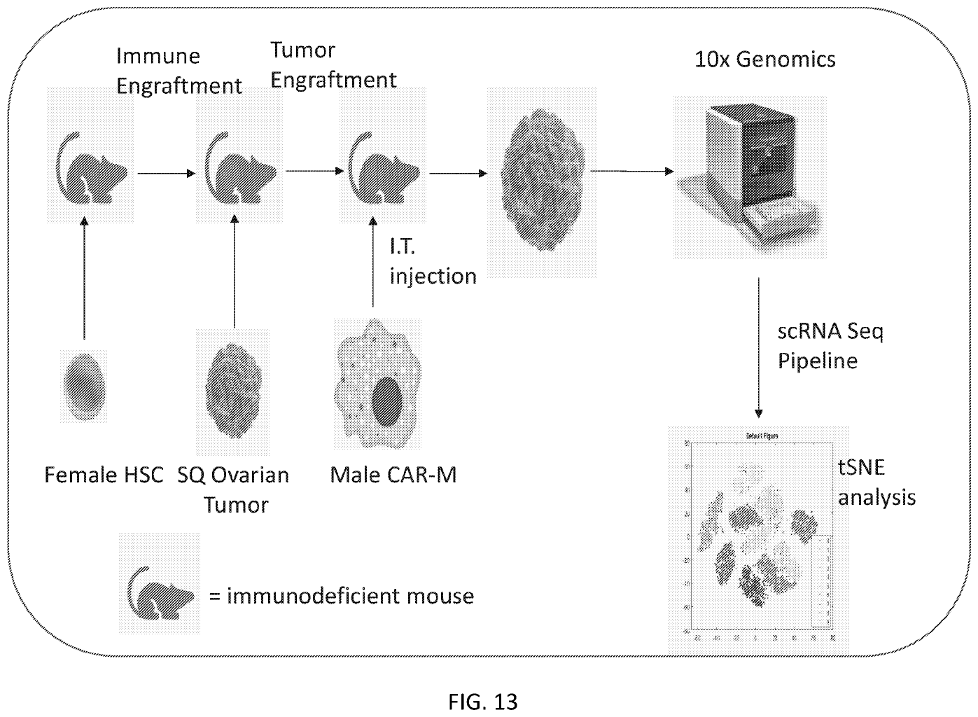

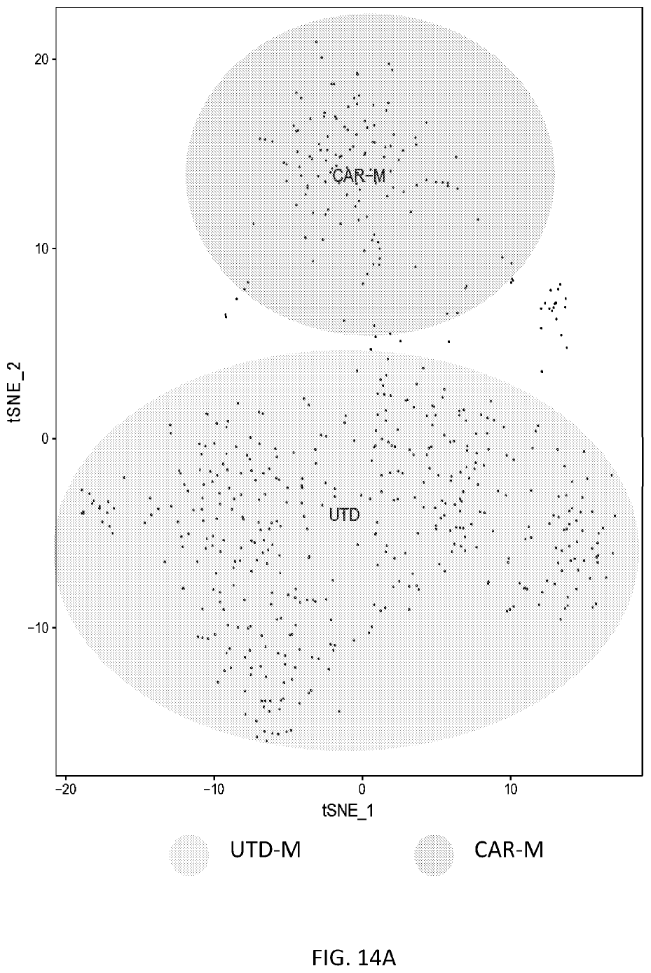

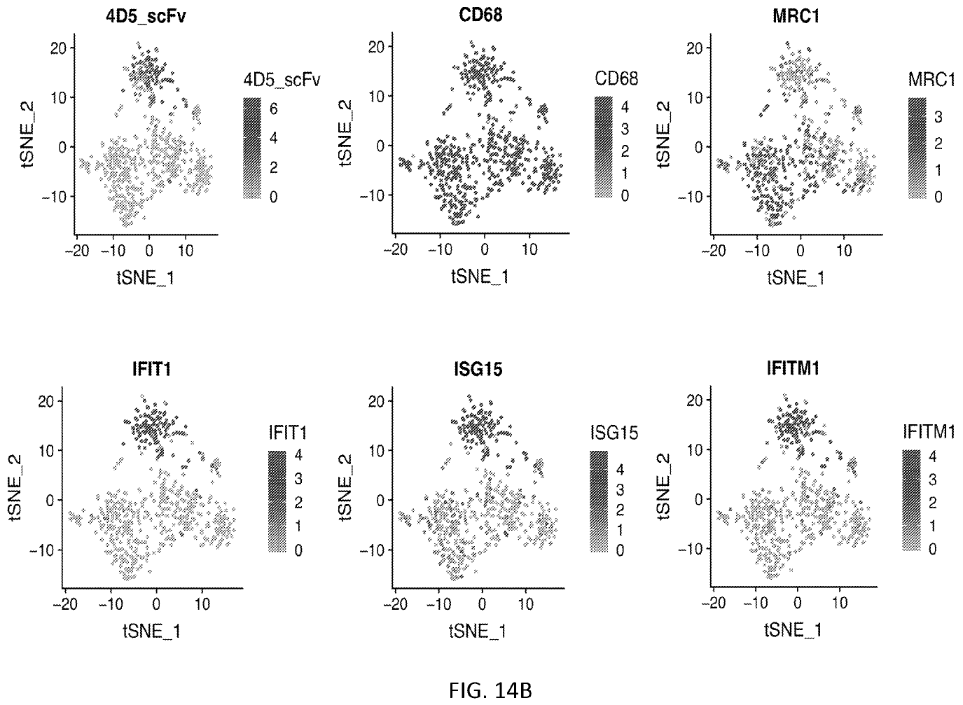

NOD scid gamma (NSG) immunodeficient mice were humanized with CD34+ human female hemopoietic stem cells. After engraftment was confirmed, ovarian cancer cells were engrafted subcutaneously in the flank of the mice (FIG. 13). After tumor engraftment and growth was visualized, human male control untransduced (UTD) or CAR-macrophages were injected intratumorally. Tumors were harvested and subject to single cell RNA sequencing (scRNA seq) using the 10× genomics pipeline. Single-cell RNA sequencing analysis was then performed on control UTD or CAR macrophages after extraction from a tumor xenograft from a humanized mouse (FIG. 14A). The phenotypes of the control (UTD) and CAR macrophages were directly compared (FIG. 14B). CAR macrophages expressed the CAR (positive control gene, 4D5 scFv). All macrophages expressed CD68, a pan-macrophage marker. Only UTD macrophages expressed the M2 marker MRC1. Only CAR macrophages expressed the M1 markers IFIT1, ISG15, and IFITM1. These data show that CAR-M maintained their M1 phenotype after several days in an immunosuppressive, humanized, tumor microenvironment.

### Example 9—CAR-M-Treated Tumor Microenvironment (TME) Differs from Control TME

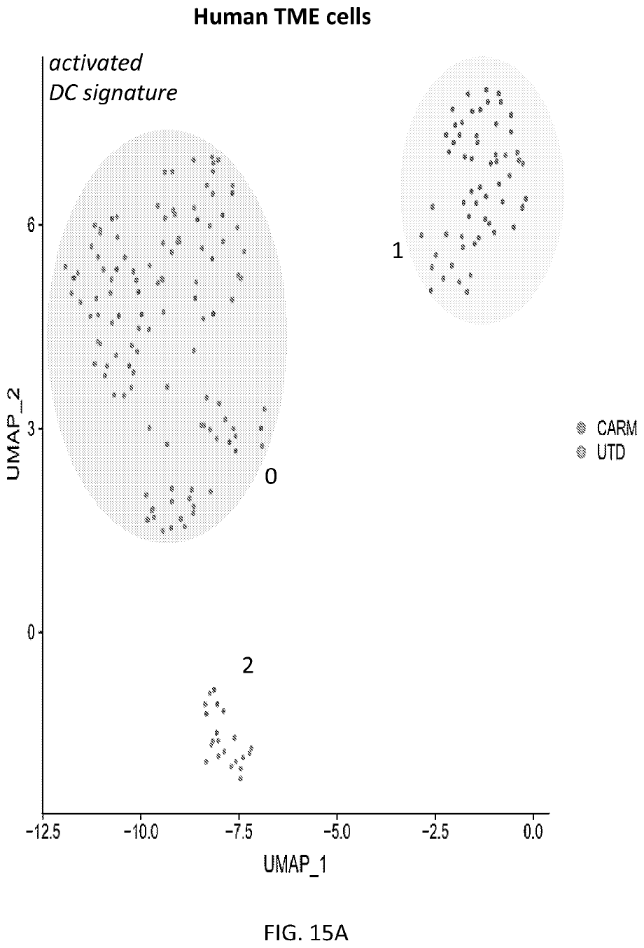

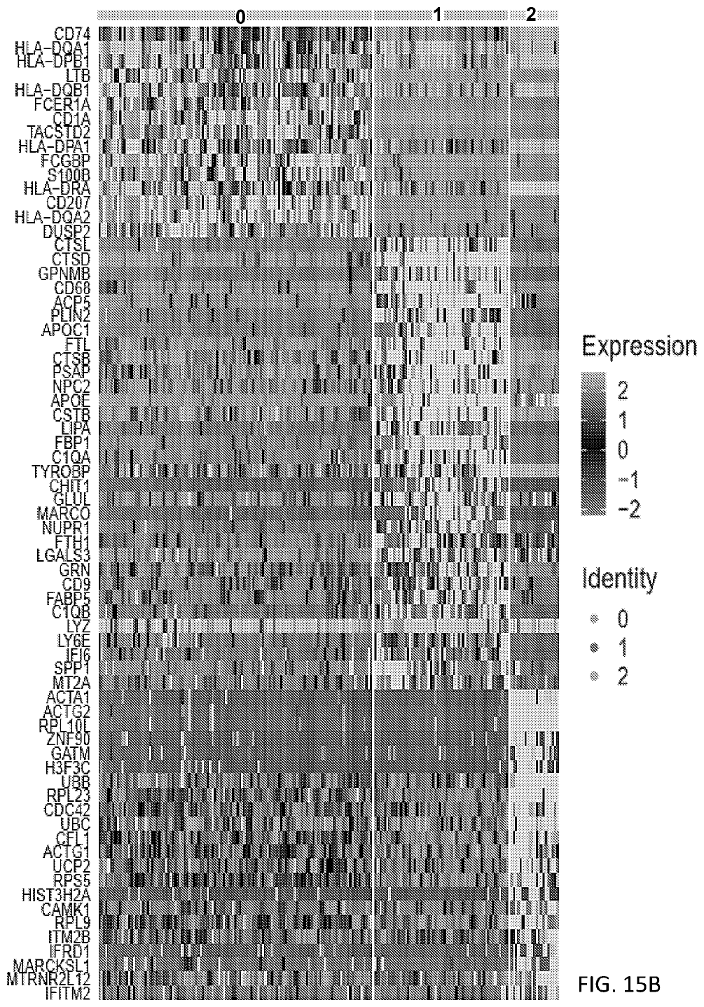

In order to further assess whether the presence of CAR-M cells in a tumor microenvironment can have an effect on other endogenous cells, subsequent single cell RNA sequencing studies were performed on monocytes isolated from xenograft tumors grown in humanized mice and treated with CAR-M cells. These results demonstrated that the tumor microenvironment (TME) of CAR-M-treated tumors was augmented (FIG. 15A). Specifically, CAR-M-treated tumors showed an increase in cells that expressed an activated dendritic cell-like (DC) signature (FIG. 15B). The presence of activated DCs in a TME is associated with favorable outcomes in patients.

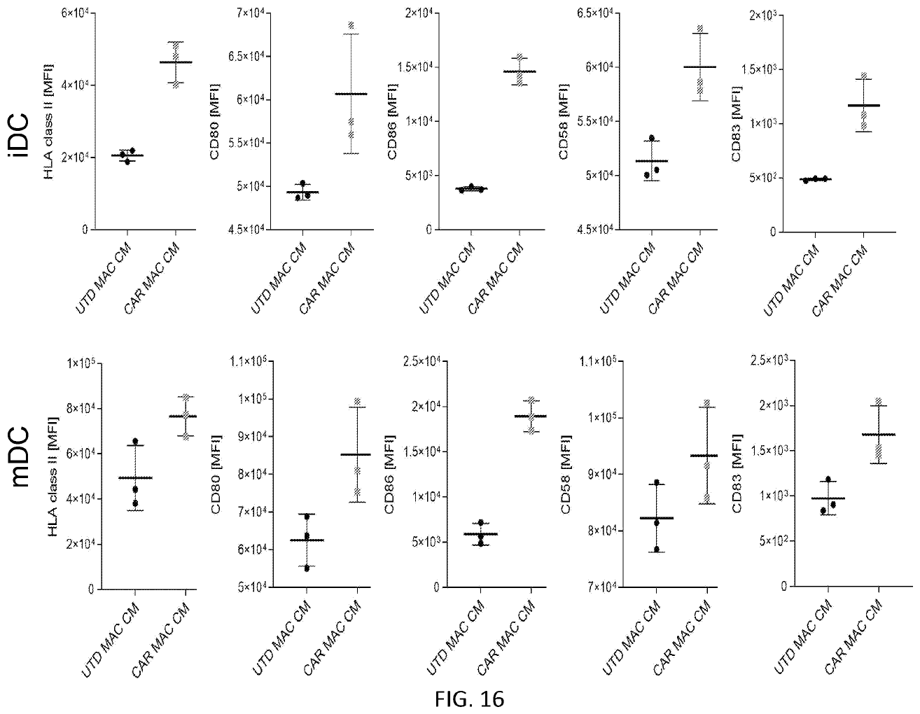

To determine whether CAR-M cells could directly influence the maturation of dendritic cells, additional studies were conducted wherein dendritic cells were first differentiated from freshly isolated monocytes by in vitro culture in media supplemented with GM-CSF and IL-4 for 9 days. Immature dendritic cells were then removed and maturation was induced by adding fresh media supplemented with GM-CSF, IL-4, and TNFα for 48 hours. Conditioned media from CAR-M or UTD macrophages was then added to the cells for an additional 48 hours followed by staining for common phenotype markers by FACS (FIG. 16). Results showed that both immature (iDCs) and mature (mDCs) dendritic cells exposed to CAR-M conditioned media had high expression of markers associated with DC maturation and function including HLA class II, CD80, CD86, CD58, and CD83 as compared to DCs cultured in UTD-conditioned media. In total, these results demonstrated that CAR-M are able to influence the phenotype of other APC populations within the tumor microenvironment, including DCs. Without wishing to be bound by theory, these results further suggest that in addition to their direct cytotoxic function, another benefit of CAR-M treatment would be improved priming of anti-tumor T cell responses as a result of enhanced dendritic cell maturation.

### Other Embodiments

The recitation of a listing of elements in any definition of a variable herein includes definitions of that variable as any single element or combination (or sub combination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for enhancing antigen presentation in a cell, the method comprising
transforming an antigen presenting cell such that the transformed antigen presenting cell includes at least one exogenous nucleic acid molecule encoding a chimeric antigen receptor (CAR);
wherein said transforming results in an increase in the antigen presenting ability of the cell as compared to a cell of the same type not having been so transformed, wherein the enhancement of antigen presenting ability is or comprises one or more of: enhanced CD8+ T cell activation, enhanced CD8+ T cell proliferation, enhanced CD8+ T cell activity, enhanced CD4+ T cell activation, enhanced CD4+ T cell proliferation, enhanced CD4+ T cell activity, enhanced NK cell activation, enhanced NK cell proliferation, and enhanced NK cell activity.

2. The method of claim 1, wherein the transformation comprises transduction with a virus or viral vector comprising at least one exogenous nucleic acid molecule encoding a chimeric antigen receptor (CAR).

3. The method of claim 1, wherein the antigen presenting cell is selected from a primary cell, a macrophage, a dendritic cell, a monocyte or a B cell.

4. The method of claim 2, wherein the virus or viral vector is an adenovirus, a lentivirus, an adeno-associated virus, or a foamy virus.

5. The method of claim 1, wherein the at least one exogenous nucleic acid molecule encodes at least one domain of a CAR selected from an antigen binding domain, a transmembrane domain, and an intracellular domain.

6. The method of claim 1, wherein the at least one exogenous nucleic acid molecule encodes two or more domains of a CAR selected from an antigen binding domain, a transmembrane domain, and an intracellular domain.

7. The method of claim 1, wherein the at least one exogenous nucleic acid molecule encodes each of an antigen binding domain, a transmembrane domain, and an intracellular domain of a CAR.

8. The method of claim 5, wherein the antigen binding domain of the CAR comprises an antibody selected from the group consisting of a monoclonal antibody, a polyclonal antibody, a synthetic antibody, a human antibody, a humanized antibody, a single domain antibody, a single chain variable fragment, and an antigen-binding fragment thereof.

9. The method of claim 8, wherein the antigen binding domain is selected from the group consisting of an anti-CD19 antibody, an anti-HER2 antibody, an anti-mesothelin antibody or a fragment thereof.

10. The method of claim 5, wherein the intracellular domain is or comprises the intracellular domain of a stimulatory or co-stimulatory molecule.

11. The method of claim 5, wherein the intracellular domain of the CAR comprises dual signaling domains.

12. The method of claim 5, further comprising administering the transduced cells to a patient in need thereof.

13. The method of claim 12, wherein the patient is suffering from one or more of a cancer, a viral infection, a bacterial infection, a parasitic infection, fibrosis, atherosclerosis, and a neurodegenerative disease.

14. The method of claim 1, wherein the antigen presenting cell is induced into an M1 phenotype prior to the transforming step.

15. The method of claim 1, wherein the antigen presenting cell is induced into an M0 phenotype prior to the transforming step.

16. The method of claim 1, wherein the antigen presenting cell exhibits an M1 phenotype prior to the transforming step.

17. The method of claim 1, wherein the antigen presenting cell exhibits an M0 phenotype prior to the transforming step.

18. A pharmaceutical composition comprising a cell which has been transformed according to the method of claim 1, wherein the cell exhibits an increase in the antigen presenting ability of the cell as compared to a cell of the same type not having been so transformed, and wherein the enhancement of antigen presenting ability is or comprises one or more of: enhanced T cell activation, enhanced T cell proliferation, and enhanced T cell activity.

19. A method for converting one or more endogenous antigen presenting cells (APCs) to a classically activated phenotype, the method comprising:
exposing the one or more endogenous APCs to one or more exogenous APCs that have been transformed such that the transformed APCs include at least one exogenous nucleic acid molecule encoding a chimeric antigen receptor (CAR).

20. The method of claim 19, wherein the transformation comprises transduction with a virus or viral vector comprising at least one exogenous nucleic acid molecule encoding a chimeric antigen receptor (CAR).

21. The method of claim 19, wherein the one or more endogenous APCs comprise monocytes, macrophages and/or dendritic cells.

22. The method of claim 19, wherein the one or more transformed exogenous APCs comprise macrophages.

23. The method of claim 19, wherein the classically activated phenotype comprises macrophages exhibiting an M1 phenotype.

24. The method of claim 23, wherein at least some of the endogenous macrophages exhibited an M2 phenotype prior to the exposing step.

25. The method of claim 19, wherein the classically activated phenotype comprises increased expression of one or more genes associated with interferon signaling, neuroinflammation signaling, Th1 development, iNOS signaling, death receptor signaling, apoptosis signaling, dendritic cell maturation, inflammasome pathway, activation of IRF by cytosolic pattern recognition receptors, RIG-1-like receptor signaling in antiviral innate immunity, cytotoxic T lymphocyte-mediated apoptosis, JAK1/JAK2/TYK2 interferon signaling, GM-CSF signaling, IL-8 signaling, acute phase response signaling, IL-1 signaling, and/or CD40 signaling.

26. The method of claim 25, wherein the genes involved in interferon signaling are selected from a list comprising BAK1, BAX, BCL2, IFI35, IFI6, IFIT1, IFIT3, IFITM2, IFITM3, IFNAR2, IFNGR2, IRF9, ISG15, OAS1, PTPN2, STAT1, STAT2, and TYK2.

27. The method of claim 25, wherein the genes involved in neuroinflammation signaling are selected from a list comprising ACVR1, APH1A, B2M, BACE2, BCL2, BIRC3, BIRC5, CASP3, CASP8, CCL5, CD80, CFLAR, CREBBP, FAS, FOS, GLS, GLUL, GRIN2D, HLA-A, HLA-DQA1, HLA-E, HLA-F, ICAM1, IFNGR2, IKBKB, IRF7, JAK3, MYD88, NCSTN, NFATC2, PIK3R2, PIK3R5, PLA2G12A, PLA2G4A, PPP3CA, PSEN1, S100B, SLC1A3, STAT1, TBK1, TGFBR1, TRAF3, TYK2, and XIAP.

28. The method of claim 25, wherein the genes involved in Th1 development are selected from a list comprising APH1A, CD274, CD80, HLA-A, HLA-DQA1, ICAM1, IFNGR2, JAK3, MAP2K6, NCSTN, NFATC2, NFIL3, PIK3R2, PIK3R5, PSEN1, RUNX3, SOCS3, STAT1, STAT3, STAT4, and TYK2.

29. The method of claim 25, wherein the genes involved in iNOS signaling are selected from a list comprising CREBBP, FOS, HMGA1, IFNGR2, IKBKB, JAK3, MYD88, STAT1, and TYK2.

30. The method of claim 25, wherein the genes involved in death receptor signaling are selected from a list comprising ACIN1, ACTA2, ACTB, ACTG1, APAF1, ARHGDIB, BCL2, BIRC3, CASP10, CASP2, CASP3, CASP7, CASP8, CFLAR, CYCS, DFFA, FAS, HSPB1, IKBKB, MAP4K4, PARP1, PARP10, PARP12, PARP14, PARP4, PARP6, PARP8, PARP9, SPTAN1, TBK1, TNFRSF21, and XIAP.

31. The method of claim 25, wherein the genes involved in apoptosis signaling are selected from a list comprising ACIN1, APAF1, BAK1, BAX, BCL2, BCL2A1, BCL2L11, BIRC3, CAPNS1, CASP10, CASP2, CASP3, CASP7, CASP8, CDK1, CYCS, DFFA, FAS, IKBKB, MAP4K4, MCL1, MRAS, NRAS, PARP1, PRKCA, RAP1A, RAP2A, SPTAN1, and XIAP.

32. The method of claim 25, wherein the genes involved in dendritic cell maturation are selected from a list comprising B2M, CCR7, CD80, CD83, COL5A3, CREBBP, FCER1G, FCGR1A, FSCN1, HLA-A, HLA-DQA1, HLA-E, HLA-F, ICAM1, IKBKB, IL15, MYD88, PIK3R2, PIK3R5, PLCB3, RELB, STAT1, STAT2, and STAT4.

33. The method of claim 25, wherein the genes involved in the inflammasome pathway are selected from a list comprising AIM2, CASP8, CTSB, MYD88, and NLRP1.

34. The method of claim 25, wherein the genes involved in the activation of IRF by cytosolic pattern recognition receptors are selected from a list comprising APAF1, B2M, BCL2, CASP3, CASP7, CASP8, CYCS, DFFA, FAS, FCER1G, HLA-A, HLA-E, and HLA-F.

35. The method of claim 25, wherein the genes involved in the role of RIG-like receptors in antiviral innate immunity are selected from a list comprising CASP10, CASP8, CREBBP, DDX58, DHX58, EP300, IFIH1, IKBKB, IRF7, MAVS, TBK1, and TRAF3.

36. The method of claim 25, wherein the genes involved in cytotoxic T lymphocyte-mediated apoptosis of target cells are selected from a list comprising APAF1, B2M, BCL2, CASP3, CASP7, CASP8, CYCS, DFFA, FAS, FCER1G, HLA-A, HLA-E, and HLA-F.

37. The method of claim 25, wherein the genes involved in the role of JAK1, JAK2, and TYK2 in interferon signaling are selected from a list comprising IFNAR2, IFNGR2, PTPN2, STAT1, STAT2, STAT3, and TYK2.

38. The method of claim 25, wherein the genes involved in GM-CSF signaling are selected from a list comprising BCL2A1, CAMK2B, CCND1, HCK, MRAS, NRAS, PIK3R2, PIK3R5, PIM1, PPP3CA, PRKCB, PTPN11, RAP1A, RAP2A, STAT1, and STAT3.

39. The method of claim 25, wherein the genes involved in IL-8 signaling are selected from a list comprising BAX, BCL2, CCND1, CCND3, CSTB, CXCR1, CXCR2, EIF4EBP1, FOS, GNA12, GNA13, GNB1, GNG12, GNG2, HBEGF, ICAM1, IKBKB, IQGAP1, ITGB5, LASP1, LIMK2, MAP4K4, MRAS, NRAS, PIK3R2, PIK3R5, PLD2, PRKCA, PRKCB, RAC2, RAP1A, RAP2A, RHOA, RHOBTB1, RHOT1, and VEGFA.

40. The method of claim 25, wherein the genes involved in acute phase response signaling are selected from a list comprising C1S, FOS, IKBKB, MAP2K3, MAP2K6, MRAS, MYD88, NRAS, PDPK1, PIK3R2, PTPN11, RAP1A, RAP2A, SERPINE1, SOCS3, and STAT3.

41. The method of claim 25, wherein the genes involved in IL-1 signaling are selected from a list comprising ADCY1, ADCY3, ADCY6, FOS, GNA12, GNA13, GNB1, GNG12, GNG2, IKBKB, MAP2K3, MAP2K6, MRAS, MYD88, PRKAR2A, PRKAR2B, and TOLLIP.

42. The method of claim 25, wherein the genes involved in CD40 signaling are selected from a list comprising FOS, ICAM1, IKBKB, JAK3, MAP2K3, MAP2K6, MAPKAPK2, PIK3R2, PIK3R5, STAT3, TNFAIP3, TRAF1, TRAF3, and TRAF5.

43. The method of claim 25, wherein the increased expression of one or more genes comprises increased expression of one or both of CD80 and CD86.

44. The method of claim 19, wherein the endogenous APCs are or comprise tumor-associated macrophages.

45. A method of killing tumor cells in a patient, the method comprising:
transforming one or more antigen presenting cells (APCs),
wherein transformed APCs comprise a chimeric antigen rector (CAR), and
administering the one or more transformed APCs to a patient;
wherein the one or more transformed APCs are able to kill tumor cells in the patient.

46. The method of claim 45, wherein transforming one or more APCs comprises transducing the one or more APCs with a virus or viral vector comprising at least one exogenous nucleic acid molecule encoding a CAR.

47. The method of claim 45, wherein the one or more transformed APCs are monocytes, macrophages and/or dendritic cells.

48. The method of claim 47, wherein the macrophages exhibit an M1 phenotype after the transformation step.

49. The method of claim 45, wherein killing tumor cells in a patient comprises reducing tumor size in the patient.

50. The method of claim 45, wherein a tumor microenvironment (TME) in the patient is altered after administration of the one or more transduced APCs to the patient.

51. The method of claim 50, wherein an altered TME comprises one or more of:
recruitment of activated myeloid cells, conversion of suppressive macrophages toward classically activated macrophages, recruitment of natural killer (NK) cells, activation of NK cells, recruitment of T cells, activation of T cells, depletion of tumor-associated macrophages, conversion of myeloid-derived suppressor cells (MDSCs), depletion of MDSCs, increased expression of pro-inflammatory cytokines, a decrease in anti-inflammatory cytokines, an increase in pro-inflammatory cells, a decrease in anti-inflammatory cells, and an increased amount of activated dendritic cells, relative to a TME prior to administration of the one or more transduced APCs to the patient.

52. The method of claim 50, wherein the TME is sampled via a process comprising biopsy of a tumor.

53. The method of claim 45, wherein the one or more modified APCs are able to kill the tumor cells in the presence of macrophages exhibiting an M2 phenotype.

54. The method of claim 45, wherein the one or more modified APCs maintain the ability to kill the tumor cells while in the presence of an inhibitory TME for a period of time.

55. The method of claim 54, wherein an inhibitory TME comprises the presence of one or more immunosuppressive cells selected from: tumor-associated macrophages, T_{reg} cells, B_{reg} cells, MDSCs, and cancer-associated fibroblasts.
